(19) United States
(12) Patent Application Publication
(10) Pub. No.: US 2024/0308994 A1
(43) Pub. Date: Sep. 19, 2024

(54) ISOXAZOLE DERIVATIVE OR PHARMACEUTICALLY ACCEPTABLE SALT THEREOF AND USE THEREOF

(71) Applicant: INDUSTRY-UNIVERSITY COOPERATION FOUNDATION HANYANG UNIVERSITY ERICA CAMPUS, Ansan-si (KR)

(72) Inventors: Jung-Mi HAH, Seoul-si (KR); Hyunwook CHO, Ansan-si (KR); Jihyun BAEK, Ansan-si (KR); Hyejin KIM, Ansan-si (KR); Joonhong JUN, Ansan-si (KR)

(73) Assignee: INDUSTRY-UNIVERSITY COOPERATION FOUNDATION HANYANG UNIVERSITY ERICA CAMPUS, Ansan-si (KR)

(21) Appl. No.: 18/593,673

(22) Filed: Mar. 1, 2024

Related U.S. Application Data
(63) Continuation-in-part of application No. PCT/KR22/18484, filed on Nov. 22, 2022.

(30) Foreign Application Priority Data
Nov. 24, 2021 (KR) 10-2021-0163159

(51) Int. Cl. C07D 413/12; A61K 31/422; A61K 31/454; A61K 31/4709; A61K 31/496; A61K 31/506; A61K 31/5377; A61K 31/55; C07D 261/18; C07D 413/14

(52) U.S. Cl. CPC C07D 413/12; A61K 31/422; A61K 31/454; A61K 31/4709; A61K 31/496; A61K 31/506; A61K 31/5377; A61K 31/55; C07D 261/18; C07D 413/14

(57) ABSTRACT

The present invention relates to an isoxazole derivative or a pharmaceutically acceptable salt thereof, and to a composition comprising said derivative as an active ingredient for preventing or treating diseases associated with CSF-1R. The isoxazole derivative of the present invention inhibits CSF-1R, and therefore can be used as a disease associated with CSF-1R for preventing or treating degenerative brain diseases.

### CROSS-REFERENCE TO RELATED APPLICATIONS

This application is a Continuation-in-Part Application of International Application No. PCT/KR2022/018484, filed on Nov. 22, 2022, which claims priority to Korean Patent Application No. 10-2021-0163159, filed on Nov. 24, 2021, the disclosures of which are incorporated by reference herein their entireties.

### TECHNICAL FIELD

The present invention relates to isoxazole derivatives or pharmaceutically acceptable salts thereof and use thereof.

### BACKGROUND ART

Ligand stimulation of FMS tyrosine phosphorylation regulates survival, proliferation and differentiation of monocyte/macrophage lineages. Macrophage, microglia, and osteoclast cells play an important role in the inflammatory process, and tumor-associated macrophages are recognized as a driving force for tumor progression and metastasis, chemotherapy, and radiotherapy. Thus, inhibition of FMS kinase activity has been studied with the potential as a therapeutic agent for bone bone lysis and inflammation as well as cancer promoted by macrophages.

Aging is known as a major risk factor for the structure and function of the nervous system, and it is necessary to overcome these detrimental effects of degenerative nerves associated with aging. The peripheral nerves of 24-month-old C57BL/6 mice exhibit the same pathological changes as those in the human elderly, but there are no such changes in 12-month-old adult mice. Specifically, the nerve fibers show demyelination, remyelination, and axonal lesion. In addition, in aging mice, nerve muscle junctions show typical feature to dying nerve disorders, which is accompanied by decreased muscle strength and increased number of macrophages. An equivalent profile can also be found in peripheral neurobiopsies of the elderly.

To examine the pathological effects of the macrophages in aging mice, when the cells were selectively treated continuously for 6 months by applying an FMS inhibitor, the number of mouse macrophages was decreased by 70% without side effects before the degenerative changes occurred in 18-month-old mice, and the nerve structure was improved, and also muscular strength was maintained (J Neurosci. 2018 May 9; 38(19):4610-4620).

Degenerative changes caused by the aging of peripheral nerves are driven by macrophages, and these findings may open the way to treat the degeneration of the peripheral nervous system of the elderly by targeting the macrophages and may lead to a reduction in the debilities of the elderly, an improvement in mobility, and ultimately an improvement in quality of life.

### DISCLOSURE

### Technical Problem

The present disclosure is directed to providing an isoxazole derivative having an CSF-1R inhibitory activity or a pharmaceutically acceptable salt thereof.

It is another object of the present invention to provide a pharmaceutical composition for preventing or treating CSF-1R-related diseases, comprising the isoxazole derivatives or pharmaceutically acceptable salts thereof as an active ingredient.

It is another object of the present invention to provide a food composition for preventing or ameliorating CSF-1R-related diseases, comprising the isoxazole derivatives or pharmaceutically acceptable salts thereof as an active ingredient.

Further, It is another object of the present invention to provide a cosmetic composition for preventing or alleviating a CSF-1R-related disease, which includes the isoxazole derivative or a pharmaceutically acceptable salt thereof as an active ingredient.

However, technical problems to be solved by the present invention are not limited to the above-mentioned problems, and other problems not mentioned can be clearly understood by skilled people in the art in the art from the following description.

### Technical Solution

In order to achieve the object, the present invention provides an isoxazole derivative represented by the following [Chemical Formula 1] or a racemate, isomer, solvate or pharmaceutically acceptable salt thereof:

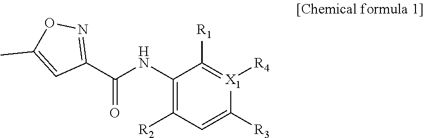

In Chemical Formula 1, X₁ is carbon or nitrogen, R₁ is a C₁ to C₆ linear or cyclic alkyl group, a C₆ to C₁₂ heteroaryl group, a substituted or unsubstituted C₄ to C₈ cycloalkene group, or hydrogen, R₂ is a C₁ to C₆ alkyl group or hydrogen, R₃ is a C₄ to C₈ heterocycloamine, a substituted or unsubstituted C₄ to C₈ heterocyclic ring, or hydrogen, R₄ is

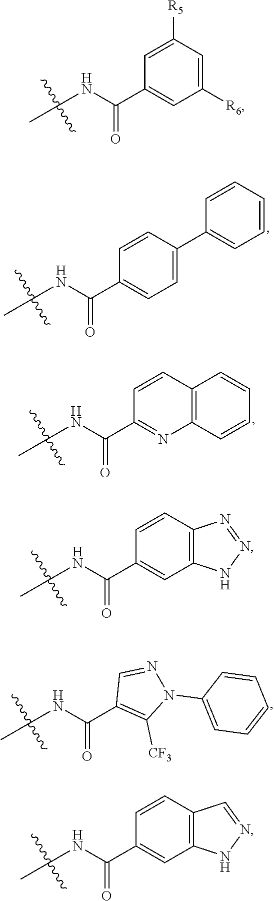

or hydrogen, R₅ is —CF₃, a substituted or unsubstituted C₃ to C₁₂ heteroaryl group, or halogen, and R₆ is a substituted or unsubstituted C₃ to C₁₂ aryl group, a substituted or unsubstituted C₃ to C₁₂ heteroaryl group, a substituted or unsubstituted C₃ to C₈ heterocyclic ring, a substituted C₄ to C₆ heterocyclooxy group, a substituted C₁ to C₆ alkyl group, or a substituted heterocycloamine group.

In one embodiment of the present invention provides R₁ in the Chemical Formula 1 is —CH₃,

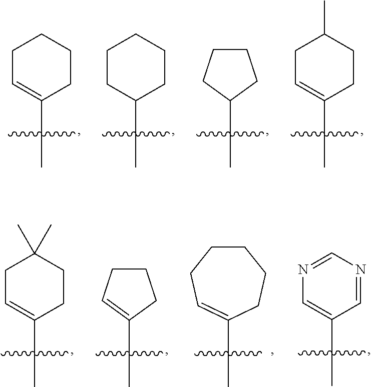

or hydrogen.

In another embodiment of the present invention, the heterocyclic ring of [Chemical Formula 1] may be one or more selected from the group consisting of piperazine, piperidine, piperidinium, pyrrolidine, azepan and morpholine, and the heteroaryl may be imidazole or pyrimidine.

In another embodiment of the present invention, the compound of [Chemical Formula 1] is at least one selected from groups consisting of 5-methyl-N-(2-methyl-5-(3-(4-methylpiperazin-1-yl)-5-(trifluoromethyl)benzamido)phenyl)isoxazole-3-carboxamide(5a);
5-methyl-N-(2-methyl-5-(3-(4-methyl-1H-imidazol-1-yl)-5-(trifluoromethyl)benzamido) phenyl)isoxazole-3-carboxamide(5b);
   5-methyl-N-(2-methyl-5-(3-morpholino-5-(trifluoromethyl)benzamido)phenyl)isoxazole-3-carboxamide(5c);
5-methyl-N-(2-methyl-5-(3-(2-methyl-1H-imidazol-1-yl)-5-(trifluoromethyl)benzamido) phenyl)isoxazole-3-carboxamide(5d);
5-methyl-N-(2-methyl-5-(3-(5-methyl-1H-imidazol-1-yl)-5-(trifluoromethyl)benzamido) phenyl)isoxazole-3-carboxamide(5e);
N-(5-([1,1′-biphenyl]-4-carboxamido)-2-methylphenyl)-5-methylisoxazole-3-carboxamide(5f);
5-methyl-N-(2-methyl-5-(3-(2-methyl-1H-imidazol-1-yl)-5-(4-methylpiperazin-1-yl)benzamido) phenyl)isoxazole-3-carboxamide(5g);
5-methyl-N-(2-methyl-5-(3-(2-methyl-1H-imidazol-1-yl)-5-((1-methylpiperidin-4-yl)amino) benzamido)phenyl)isoxazole-3-carboxamide(5h);
5-methyl-N-(2-methyl-5-(3-((1-methylpiperidin-4-yl)amino)-5-(trifluoromethyl)benzamido) phenyl)isoxazole-3-carboxamide, 5i;
N-(5-(3-((4-Ethylpiperazin-1-yl)methyl)-5-(trifluoromethyl)benzamido)-2-methylphenyl)-5-methylisoxazole-3-carboxamide(5j);
N-(5-(3-(4-Hydroxypiperidin-1-yl)-5-(trifluoromethyl)benzamido)-2-methylphenyl)-5-methylisoxazole-3-carboxamide(5k);
(R)-5-Methyl-N-(2-methyl-5-(3-((1-methylpiperidin-3-yl)amino)-5-(trifluoromethyl)benzamido) phenyl)isoxazole-3-carboxamide(5l);
N,N,N-trimethyl-1-(3-((4-methyl-3-(5-methylisoxazole-3-carboxamido)phenyl)carbamoyl)-5-(trifluoromethyl)phenyl)pyrrolidin-3-aminium(5m);
N-(5-(3-(3-(ethylamino)pyrrolidin-1-yl)-5-(trifluoromethyl)benzamido)-2-methylphenyl)-5-methylisoxazole-3-carboxamide(5n);
5-methyl-N-(2-methyl-5-(3-((1-methylpyrrolidin-3-yl)amino)-5-(trifluoromethyl)benzamido)phenyl)isoxazole-3-carboxamide(5o);
N-(5-(3-(3-(dimethylamino)pyrrolidin-1-yl)-5-(trifluoromethyl)benzamido)-2-methylphenyl)-5-methylisoxazole-3-carboxamide(5p);
5-methyl-N-(2-methyl-5-(quinoline-2-carboxamido)phenyl)isoxazole-3-carboxamide(5q);
N-(5-(1H-benzo[d][1,2,3]triazole-6-carboxamido)-2-methylphenyl)-5-methylisoxazole-3-carboxamide(5r);
5-methyl-N-(2-methyl-5-(1-phenyl-5-(trifluoromethyl)-1H-pyrazole-4-carboxamido)phenyl)isoxazole-3-carboxamide(5s);
(N-(5-(1H-indazole-6-carboxamido)-2-methylphenyl)-5-methylisoxazole-3-carboxamide(5t);
N-(5-(3-Chlorobenzamido)-2-methylphenyl)-5-methylisoxazole-3-carboxamide(5u);
5-Methyl-N-(2-methyl-5-(3-((1-methylpiperidin-4-yl)oxy)-5-(trifluoromethyl)benzamido) phenyl)isoxazole-3-carboxamide(5v);
5-methyl-N-(2-methyl-6-((tetrahydro-2H-pyran-4-yl)amino)pyridin-3-yl)isoxazole-3-carboxamide(17b);
5-methyl-N-(5-(pyrrolidin-3-yl)-2′,3′,4′,5′-tetrahydro-[1,1′-biphenyl]-2-yl)isoxazole-3-carboxamide(60a);
5-methyl-N-(5-(piperidin-4-yl)-2′,3′,4′,5′-tetrahydro-[1,1′-biphenyl]-2-yl)isoxazole-3-carboxamide(60b);
N-(5-(azepan-4-yl)-2′,3′,4′,5′-tetrahydro-[1,1′-biphenyl]-2-yl)-5-methylisoxazole-3-carboxamide(60c);
N-(2-(Cyclopent-1-en-1-yl)-4-(piperidin-4-yl)phenyl)-5-methylisoxazole-3-carboxamide(61 b);
5-methyl-N-(4-(piperidin-4-yl)-2-(pyrimidin-5-yl)phenyl)isoxazole-3-carboxamide(62b);
5-Methyl-N-(4′-methyl-5-(piperidin-4-yl)-2′,3′,4′,5′-tetrahydro-[1,1′-biphenyl]-2-yl)isoxazole-3-carboxamide(63b);
N-(4′,4′-dimethyl-5-(piperidin-4-yl)-2′,3′,4′,5′-tetrahydro-[1,1′-biphenyl]-2-yl)-5-methylisoxazole-3-carboxamide(64b);
N-(5-(azepan-4-yl)-4′,4′-dimethyl-2′,3′,4′,5′-tetrahydro-[1,1′-biphenyl]-2-yl)-5-methylisoxazole-3-carboxamide(64c);
N-(2-cyclohexyl-4-(piperidin-4-yl)phenyl)-5-methylisoxazole-3-carboxamide(65b);
N-(2-Cyclopentyl-4-(piperidin-4-yl)phenyl)-5-methylisoxazole-3-carboxamide(66b);
5-methyl-N-(5-(1-methylpiperidin-4-yl)-2′,3′,4′,5′-tetrahydro-[1,1′-biphenyl]-2-yl)isoxazole-3-carboxamide(67b);
5-methyl-N-(5-(1-methylazepan-4-yl)-2′,3′,4′,5′-tetrahydro-[1,1′-biphenyl]-2-yl)isoxazole-3-carboxamide(67c);
N-(4′,4′-dimethyl-5-(1-methylazepan-4-yl)-2′,3′,4′,5′-tetrahydro-[1,1′-biphenyl]-2-yl)-5-methylisoxazole-3-carboxamide(68c);
N-(2-cyclohexyl-4-(1-methylpiperidin-4-yl)phenyl)-5-methylisoxazole-3-carboxamide(69b);
N-(5-(1-ethylpiperidin-4-yl)-2′,3′,4′,5′-tetrahydro-[1,1′-biphenyl]-2-yl)-5-methylisoxazole-3-carboxamide(70b);
N-(2-(Cyclopent-1-en-1-yl)-4-(1-ethylpiperidin-4-yl)phenyl)-5-methylisoxazole-3-carboxamide(71 b);
N-(5-(1-Ethylpiperidin-4-yl)-4′-methyl-2′,3′,4′,5′-tetrahydro-[1,1′-biphenyl]-2-yl)-5-methylisoxazole-3-carboxamide(72b);
N-(5-(1-ethylpiperidin-4-yl)-4′,4′-dimethyl-2′,3′,4′,5′-tetrahydro-[1,1′-biphenyl]-2-yl)-5-methylisoxazole-3-carboxamide(73b);
N-(2-cyclohexyl-4-(1-ethylpiperidin-4-yl)phenyl)-5-methylisoxazole-3-carboxamide(74b);
N-(2-Cyclopentyl-4-(1-ethylpiperidin-4-yl)phenyl)-5-methylisoxazole-3-carboxamide(75b);
5-methyl-N-(5-(1-(3-methylbut-2-en-1-yl)piperidin-4-yl)-2′,3′,4′,5′-tetrahydro-[1,1′-biphenyl]-2-yl)isoxazole-3-carboxamide(76b);
N-(5-(1-(cyclopropylmethyl)piperidin-4-yl)-4′-methyl-2′,3′,4′,5′-tetrahydro-[1,1′-biphenyl]-2-yl)-5-methylisoxazole-3-carboxamide(77b);
N-(5-(1-(cyclopropylmethyl)piperidin-4-yl)-4′,4′-dimethyl-2′,3′,4′,5′-tetrahydro-[1,1′-biphenyl]-2-yl)-5-methylisoxazole-3-carboxamide(78b);
1,1-dimethyl-4-(6-(5-methylisoxazole-3-carboxamido)-2′,3′,4′,5′-tetrahydro-[1,1′-biphenyl]-3-yl)piperidin-1-ium(79b);
1,1-dimethyl-4-(6-(5-methylisoxazole-3-carboxamido)-2′,3′,4′,5′-tetrahydro-[1,1′-biphenyl]-3-yl)azepan-1-ium(79c);
4-(4′,4′-dimethyl-6-(5-methylisoxazole-3-carboxamido)-2′,3′,4′,5′-tetrahydro-[1,1′-biphenyl]-3-yl)-1,1-dimethylazepan-1-ium(80c);
4-(3-cyclohexyl-4-(5-methylisoxazole-3-carboxamido)phenyl)-1,1-dimethylpiperidin-1-ium(81 b);
1,1-diethyl-4-(6-(5-methylisoxazole-3-carboxamido)-2′,3′,4′,5′-tetrahydro-[1,1′-biphenyl]-3-yl)piperidin-1-ium(82b);
N-(5-(1-acetylpiperidin-4-yl)-2′,3′,4′,5′-tetrahydro-[1,1′-biphenyl]-2-yl)-5-methylisoxazole-3-carboxamide(83b);
N-(5-(1-(dimethylglycyl)piperidin-4-yl)-2′,3′,4′,5′-tetrahydro-[1,1′-biphenyl]-2-yl)-5-methylisoxazole-3-carboxamide(84b);
5-methyl-N-(5-(2,2,6,6-tetramethylpiperidin-4-yl)-2′,3′,4′,5′-tetrahydro-[1,1′-biphenyl]-2-yl)isoxazole-3-carboxamide(92a);
5-methyl-N-(4′-methyl-5-(2,2,6,6-tetramethylpiperidin-4-yl)-2′,3′,4′,5′-tetrahydro-[1,1′-biphenyl]-2-yl)isoxazole-3-carboxamide(92b);
N-(4′,4′-dimethyl-5-(2,2,6,6-tetramethylpiperidin-4-yl)-2′,3′,4′,5′-tetrahydro-[1,1′-biphenyl]-2-yl)-5-methylisoxazole-3-carboxamide, 92c; and N-(2-(cyclohept-1-en-1-yl)-4-(2,2,6,6-tetramethylpiperidin-4-yl)phenyl)-5-methylisoxazole-3-carboxamide(92d).

In another embodiment of the present invention, the isoxazole derivative may inhibit CSF-1R.

In addition, the present invention provides a composition for preventing or treating CSF-1R-related diseases, which includes the isoxazole derivatives or pharmaceutically acceptable salts thereof as an active ingredient.

In addition, the present invention provides a method for preventing or treating CSF-1R-related diseases, comprising a step of administering the isoxazole derivatives or pharmaceutically acceptable salts thereof to an individual.

In addition, the present invention provides a use of the isoxazole derivative or a pharmaceutically acceptable salt thereof for preparing a medicament for preventing or treating CSF-1R-related diseases.

In another embodiment of the present invention, the CSF-1R-related disease is a degenerative brain disease such as Alzheimer's disease, Parkinson's disease, Huntington's disease, prosencephaly, microcephaly, cerebral palsy, congenital hydrocephalus, Wilson disease, dementias, multi infarct dementia, Frontotemporal dementia, pseudo-dementia, Motor neuron diseases, spinocerebellar ataxia, spinal muscular atrophy.

### Advantageous Effects

The present invention provides an N-Aryl-5-methylisoxazole-3-carboxamide derivative or a pharmaceutically acceptable salt thereof, and a composition for preventing or treating diseases associated with CSF-1R containing the derivative as an active ingredient.

The N-Aryl-5-methylisoxazole-3-carboxamide of the present invention inhibits CSF-1R which regulates macrophages that drive neurodegenerative diseases, and thus can be useful for preventing or treating neurodegenerative diseases such as Alzheimer's dementia, Parkinson's disease, Huntington's disease, and the like.

The effects of the present invention are not limited to those mentioned above, and other effects not mentioned may be clearly understood by the skilled person in the art from the following description.

### BRIEF DESCRIPTION OF DRAWINGS

FIG. 1 shows a synthesis scheme of 5-methylisoxazole-3-carboxamide derivatives.

FIGS. 2a-2c show a synthesis scheme of Compound 60a˜84b derivatives, wherein FIG. 2a shows a preparation process of Intermediate Compound 45a˜45c, FIG. 2b shows a preparation process of Intermediate Compound 53a˜59b, and FIG. 2c shows a preparation process of Compound 60a˜84b.

FIG. 3 shows a synthesis scheme of tetramethylpiperidine-isoxazole-carboxamide derivatives.

FIG. 4 shows a result of confirming the selectivity of Compound 92b through Protein Kinase Profiling analysis.

FIG. 5 is a graph showing the change of the protein kinase profile for 50 selected protein Kinase in Comparative example 1 (“1” in FIG. 5), Example 5d (“7d” in FIG. 5), and Comparative example 2.

### BEST MODE

Ligand stimulation of FMS tyrosine phosphorylation regulates survival, proliferation and differentiation of the monocyte/macrophage lineage, and inhibition of FMS kinase activity may improve bone osteolysis and inflammation as well as degenerative changes due to aging of the peripheral nerves that may be promoted by macrophages. As an effective FMS inhibitor for this, we provide N-Aryl-5-methylisoxazole-3-carboxamide derivatives as selective CSF-1R inhibitors.

From the above results, the present invention may provide isoxazole derivatives represented by the following [Chemical Formula 1] or pharmaceutically acceptable salts thereof.

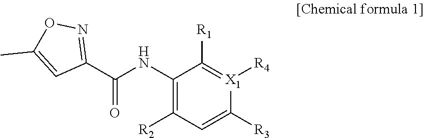

In Chemical Formula 1, X1 is carbon or nitrogen, R1 is a C1 to C6 linear or cyclic alkyl group, a C6 to C12 heteroaryl group, a substituted or unsubstituted C4 to C8 cycloalkene group, or hydrogen, R2 is a C1 to C6 alkyl group or hydrogen, R3 is a C4 to C8 heterocycloamine, a substituted or unsubstituted C4 to C8 heterocyclic group, or hydrogen, R₄ is,

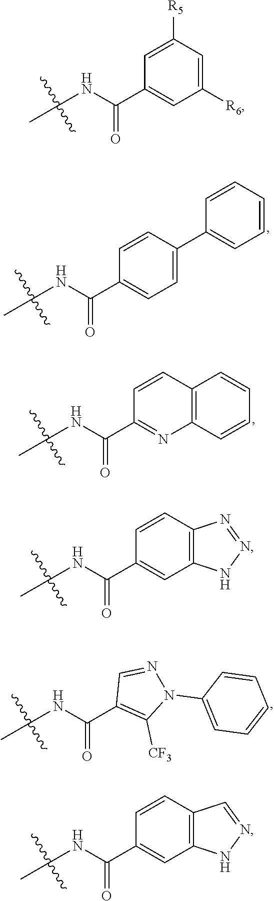

or hydrogen, R5 is —CF3, a substituted or unsubstituted C3 to C12 heteroaryl group, or halogen, and R6 is a substituted or unsubstituted C3 to C12 aryl group, a substituted or unsubstituted C3 to C12 heteroaryl group, a substituted or unsubstituted C3 to C8 heterocyclic group, a substituted C4 to C6 heterocyclooxy group, a substituted C1 to C6 alkyl group, or a substituted heterocycloamine group.

In one embodiment of the present invention, R1 of [Chemical Formula 1] may be —CH₃,

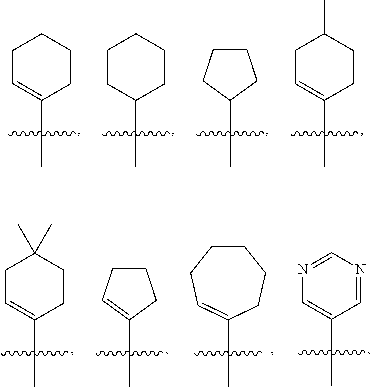

or hydrogen.

In another embodiment of the present invention, the heterocyclic ring of [Chemical Formula 1] may be one or more selected from the group consisting of piperazine, piperidine, piperidinium, pyrrolidine, azepan and morpholine, and the heteroaryl may be imidazole or pyrimidine.

In another embodiment of the present invention, the compound of [Chemical Formula 1] is at least one selected from groups consisting of 5-methyl-N-(2-methyl-5-(3-(4-methylpiperazin-1-yl)-5-(trifluoromethyl)benzamido)phenyl)isoxazole-3-carboxamide(5a);
5-methyl-N-(2-methyl-5-(3-(4-methyl-1H-imidazol-1-yl)-5-(trifluoromethyl)benzamido) phenyl)isoxazole-3-carboxamide(5b);
5-methyl-N-(2-methyl-5-(3-morpholino-5-(trifluoromethyl)benzamido)phenyl)isoxazole-3-carboxamide(5c);
5-methyl-N-(2-methyl-5-(3-(2-methyl-1H-imidazol-1-yl)-5-(trifluoromethyl)benzamido) phenyl)isoxazole-3-carboxamide(5d);
5-methyl-N-(2-methyl-5-(3-(5-methyl-1H-imidazol-1-yl)-5-(trifluoromethyl)benzamido) phenyl)isoxazole-3-carboxamide(5e);
N-(5-([1,1′-biphenyl]-4-carboxamido)-2-methylphenyl)-5-methylisoxazole-3-carboxamide(5f);
5-methyl-N-(2-methyl-5-(3-(2-methyl-1H-imidazol-1-yl)-5-(4-methylpiperazin-1-yl)benzamido) phenyl)isoxazole-3-carboxamide(5g);
5-methyl-N-(2-methyl-5-(3-(2-methyl-1H-imidazol-1-yl)-5-((1-methylpiperidin-4-yl)amino) benzamido)phenyl)isoxazole-3-carboxamide(5h);
5-methyl-N-(2-methyl-5-(3-((1-methylpiperidin-4-yl)amino)-5-(trifluoromethyl)benzamido) phenyl)isoxazole-3-carboxamide, 5i;
N-(5-(3-((4-Ethylpiperazin-1-yl)methyl)-5-(trifluoromethyl)benzamido)-2-methylphenyl)-5-methylisoxazole-3-carboxamide(5j);
N-(5-(3-(4-Hydroxypiperidin-1-yl)-5-(trifluoromethyl)benzamido)-2-methylphenyl)-5-methylisoxazole-3-carboxamide(5k);
(R)-5-Methyl-N-(2-methyl-5-(3-((1-methylpiperidin-3-yl)amino)-5-(trifluoromethyl)benzamido) phenyl)isoxazole-3-carboxamide(5l);
N,N,N-trimethyl-1-(3-((4-methyl-3-(5-methylisoxazole-3-carboxamido)phenyl)carbamoyl)-5-(trifluoromethyl)phenyl)pyrrolidin-3-aminium(5m);
N-(5-(3-(3-(ethylamino)pyrrolidin-1-yl)-5-(trifluoromethyl)benzamido)-2-methylphenyl)-5-methylisoxazole-3-carboxamide(5n);
5-methyl-N-(2-methyl-5-(3-((1-methylpyrrolidin-3-yl)amino)-5-(trifluoromethyl)benzamido)phenyl)isoxazole-3-carboxamide(5o);
N-(5-(3-(3-(dimethylamino)pyrrolidin-1-yl)-5-(trifluoromethyl)benzamido)-2-methylphenyl)-5-methylisoxazole-3-carboxamide(5p);
5-methyl-N-(2-methyl-5-(quinoline-2-carboxamido)phenyl)isoxazole-3-carboxamide(5q);
N-(5-(1H-benzo[d][1,2,3]triazole-6-carboxamido)-2-methylphenyl)-5-methylisoxazole-3-carboxamide(5r);
5-methyl-N-(2-methyl-5-(1-phenyl-5-(trifluoromethyl)-1H-pyrazole-4-carboxamido)phenyl)isoxazole-3-carboxamide(5s);
(N-(5-(1H-indazole-6-carboxamido)-2-methylphenyl)-5-methylisoxazole-3-carboxamide(5t);
N-(5-(3-Chlorobenzamido)-2-methylphenyl)-5-methylisoxazole-3-carboxamide(5u);
5-Methyl-N-(2-methyl-5-(3-((1-methylpiperidin-4-yl)oxy)-5-(trifluoromethyl)benzamido) phenyl)isoxazole-3-carboxamide(5v);
5-methyl-N-(2-methyl-6-((tetrahydro-2H-pyran-4-yl)amino)pyridin-3-yl)isoxazole-3-carboxamide(17b);
5-methyl-N-(5-(pyrrolidin-3-yl)-2′,3′,4′,5′-tetrahydro-[1,1′-biphenyl]-2-yl)isoxazole-3-carboxamide(60a);
5-methyl-N-(5-(piperidin-4-yl)-2′,3′,4′,5′-tetrahydro-[1,1′-biphenyl]-2-yl)isoxazole-3-carboxamide(60b);
N-(5-(azepan-4-yl)-2′,3′,4′,5′-tetrahydro-[1,1′-biphenyl]-2-yl)-5-methylisoxazole-3-carboxamide(60c);
N-(2-(Cyclopent-1-en-1-yl)-4-(piperidin-4-yl)phenyl)-5-methylisoxazole-3-carboxamide(61 b);
5-methyl-N-(4-(piperidin-4-yl)-2-(pyrimidin-5-yl)phenyl)isoxazole-3-carboxamide(62b);
5-Methyl-N-(4′-methyl-5-(piperidin-4-yl)-2′,3′,4′,5′-tetrahydro-[1,1′-biphenyl]-2-yl)isoxazole-3-carboxamide(63b);
N-(4′,4′-dimethyl-5-(piperidin-4-yl)-2′,3′,4′,5′-tetrahydro-[1,1′-biphenyl]-2-yl)-5-methylisoxazole-3-carboxamide(64b);
N-(5-(azepan-4-yl)-4′,4′-dimethyl-2′,3′,4′,5′-tetrahydro-[1,1′-biphenyl]-2-yl)-5-methylisoxazole-3-carboxamide(64c);
N-(2-cyclohexyl-4-(piperidin-4-yl)phenyl)-5-methylisoxazole-3-carboxamide(65b);
N-(2-Cyclopentyl-4-(piperidin-4-yl)phenyl)-5-methylisoxazole-3-carboxamide(66b);
5-methyl-N-(5-(1-methylpiperidin-4-yl)-2′,3′,4′,5′-tetrahydro-[1,1′-biphenyl]-2-yl)isoxazole-3-carboxamide(67b);
5-methyl-N-(5-(1-methylazepan-4-yl)-2′,3′,4′,5′-tetrahydro-[1,1′-biphenyl]-2-yl)isoxazole-3-carboxamide(67c);
N-(4′,4′-dimethyl-5-(1-methylazepan-4-yl)-2′,3′,4′,5′-tetrahydro-[1,1′-biphenyl]-2-yl)-5-methylisoxazole-3-carboxamide(68c);
N-(2-cyclohexyl-4-(1-methylpiperidin-4-yl)phenyl)-5-methylisoxazole-3-carboxamide(69b);
N-(5-(1-ethylpiperidin-4-yl)-2′,3′,4′,5′-tetrahydro-[1,1′-biphenyl]-2-yl)-5-methylisoxazole-3-carboxamide(70b);
N-(2-(Cyclopent-1-en-1-yl)-4-(1-ethylpiperidin-4-yl)phenyl)-5-methylisoxazole-3-carboxamide(71 b);
N-(5-(1-Ethylpiperidin-4-yl)-4′-methyl-2′,3′,4′,5′-tetrahydro-[1,1′-biphenyl]-2-yl)-5-methylisoxazole-3-carboxamide(72b);
N-(5-(1-ethylpiperidin-4-yl)-4′,4′-dimethyl-2′,3′,4′,5′-tetrahydro-[1,1′-biphenyl]-2-yl)-5-methylisoxazole-3-carboxamide(73b);
N-(2-cyclohexyl-4-(1-ethylpiperidin-4-yl)phenyl)-5-methylisoxazole-3-carboxamide(74b);
N-(2-Cyclopentyl-4-(1-ethylpiperidin-4-yl)phenyl)-5-methylisoxazole-3-carboxamide(75b);
5-methyl-N-(5-(1-(3-methylbut-2-en-1-yl)piperidin-4-yl)-2′,3′,4′,5′-tetrahydro-[1,1′-biphenyl]-2-yl)isoxazole-3-carboxamide(76b);
N-(5-(1-(cyclopropylmethyl)piperidin-4-yl)-4′-methyl-2′,3′,4′,5′-tetrahydro-[1,1′-biphenyl]-2-yl)-5-methylisoxazole-3-carboxamide(77b);
N-(5-(1-(cyclopropylmethyl)piperidin-4-yl)-4′,4′-dimethyl-2′,3′,4′,5′-tetrahydro-[1,1′-biphenyl]-2-yl)-5-methylisoxazole-3-carboxamide(78b);
1,1-dimethyl-4-(6-(5-methylisoxazole-3-carboxamido)-2′,3′,4′,5′-tetrahydro-[1,1′-biphenyl]-3-yl)piperidin-1-ium(79b);
1,1-dimethyl-4-(6-(5-methylisoxazole-3-carboxamido)-2′,3′,4′,5′-tetrahydro-[1,1′-biphenyl]-3-yl)azepan-1-ium(79c);
4-(4′,4′-dimethyl-6-(5-methylisoxazole-3-carboxamido)-2′,3′,4′,5′-tetrahydro-[1,1′-biphenyl]-3-yl)-1,1-dimethylazepan-1-ium(80c);
4-(3-cyclohexyl-4-(5-methylisoxazole-3-carboxamido)phenyl)-1,1-dimethylpiperidin-1-ium(81 b);
1,1-diethyl-4-(6-(5-methylisoxazole-3-carboxamido)-2′,3′,4′,5′-tetrahydro-[1,1′-biphenyl]-3-yl)piperidin-1-ium(82b);
N-(5-(1-acetylpiperidin-4-yl)-2′,3′,4′,5′-tetrahydro-[1,1′-biphenyl]-2-yl)-5-methylisoxazole-3-carboxamide(83b);
N-(5-(1-(dimethylglycyl)piperidin-4-yl)-2′,3′,4′,5′-tetrahydro-[1,1′-biphenyl]-2-yl)-5-methylisoxazole-3-carboxamide(84b);
5-methyl-N-(5-(2,2,6,6-tetramethylpiperidin-4-yl)-2′,3′,4′,5′-tetrahydro-[1,1′-biphenyl]-2-yl)isoxazole-3-carboxamide(92a);
5-methyl-N-(4′-methyl-5-(2,2,6,6-tetramethylpiperidin-4-yl)-2′,3′,4′,5′-tetrahydro-[1,1′-biphenyl]-2-yl)isoxazole-3-carboxamide(92b);
N-(4′,4′-dimethyl-5-(2,2,6,6-tetramethylpiperidin-4-yl)-2′,3′,4′,5′-tetrahydro-[1,1′-biphenyl]-2-yl)-5-methylisoxazole-3-carboxamide, 92c; and N-(2-(cyclohept-1-en-1-yl)-4-(2,2,6,6-tetramethylpiperidin-4-yl)phenyl)-5-methylisoxazole-3-carboxamide(92d).

In another embodiment of the present invention, the isoxazole derivative may inhibit CSF-1R.

In addition, the present invention provides a composition for preventing or treating CSF-1R-related diseases, which includes the isoxazole derivatives or pharmaceutically acceptable salts thereof as an active ingredient.

In addition, the present invention provides a method for preventing or treating CSF-1R-related diseases, comprising a step of administering the isoxazole derivatives or pharmaceutically acceptable salts thereof to an individual.

In addition, the present invention provides a use of the isoxazole derivative or a pharmaceutically acceptable salt thereof for preparing a medicament for preventing or treating CSF-1R-related diseases.

In another embodiment of the present invention, the CSF-1R-related disease is a degenerative brain disease such as Alzheimer's disease, Parkinson's disease, Huntington's disease, prosencephaly, microcephaly, cerebral palsy, congenital hydrocephalus, Wilson disease, dementias, multi infarct dementia, Frontotemporal dementia, pseudo-dementia, Motor neuron diseases, spinocerebellar ataxia, spinal muscular atrophy.

In the present invention, the term “substitution” refers to a reaction in which an atom or an atomic group included in a molecule of a compound is replaced with another atom or atomic group.

In the present invention, the term “linear form” refers to a molecule having a linear-form structure, and the linear-form structure is a chemical structure in which carbon atoms are connected in a linear shape and has a straight linear shape and a branched chain shape.

In the present invention, the term “cyclic” refers to a structure in which both ends linked in the skeleton of an organic compound are connected to form a cyclic shape.

In one embodiment of the present invention, in Chemical Formula 1, R1 may be —R1 of [Chemical Formula 1] may be —CH₃,

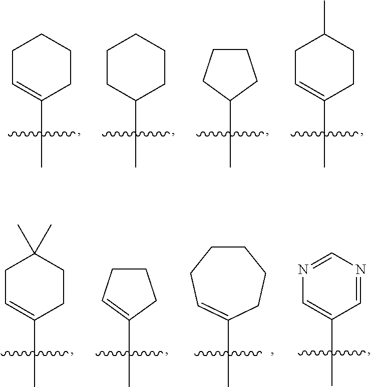

or hydrogen.

In the present invention, the heterocycle may be one or more selected from the group consisting of piperazine, piperidine, piperidinium, pyrrolidine, azepane, and morpholine, and the heteroaryl may be imidazole or pyrimidine.

More specifically, the substituted heterocycloamine group in R₃ may be an amine group substituted with tetrahydropyran

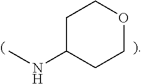

In addition, the unsubstituted heterocyclic ring of R3 may be pyrrolidine, piperidine, azepane, piperidinium, or azepanium, and the substituted heterocyclic ring may be one in which the pyrrolidine, piperidine, azepane, piperidinium, or azepanium is substituted with 1-methyl, 1-ethyl, 1-(3-methylbut-2-en-1-yl), 1-cyclopropylmethyl, 1,1-dimethyl, 1,1-diethyl, 1-acetyl, 1-dimethylglycyl, or 2,2,6,6-tetramethyl group. More specifically, the heterocyclic may be

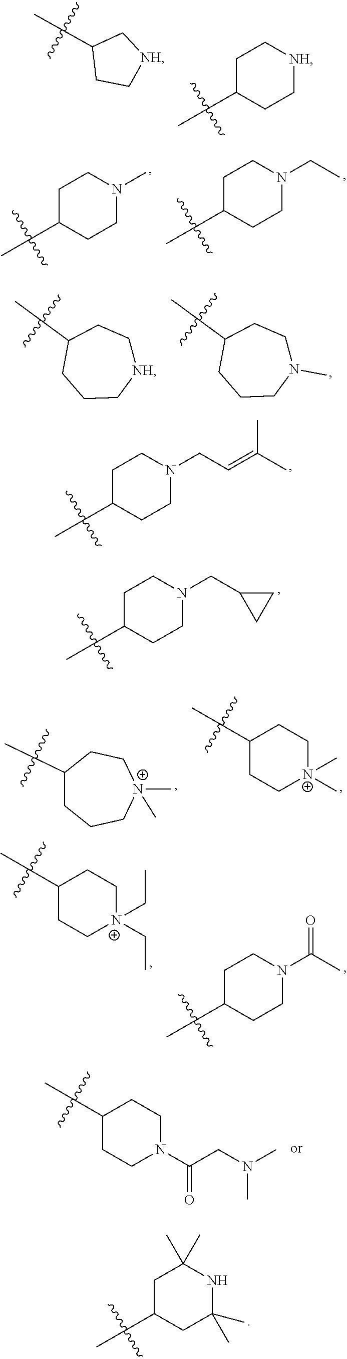

In another embodiment of the present invention, the substituted heteroaryl group in R5 or R6 may be 4-methylimidazole, 2-methylimidazole, or 5-methylimidazole, and the substituted heterocyclooxy group in R6 may be a heterocyclooxy group substituted with 1-methyl. More specifically, the heterocyclooxy group may be

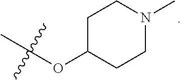 [SMILES: *OC1CCN(C)CC1]

In addition, the alkyl group substituted in R6 may be substituted with 4-ethylpiperazine, and the substituted heterocycloalkyl group may be an amine group substituted with 1-methylpyrrolidine or 1-methylpiperidine and may be specifically

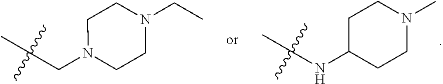

In R6, the heterocyclic ring may be piperazine, morpholine, piperidine, or pyrrolidine, the unsubstituted heterocyclic ring may be morpholine, and the substituted heterocyclic ring may be substituted with 4-methyl, 1-methyl, 4-ethyl, trimethylammonium, 4-hydroxy, 3-ethylamino, or 3-dimethylamino in the piperazine, piperidine, or pyrrolidine. More specifically, the substituted heterocyclic in

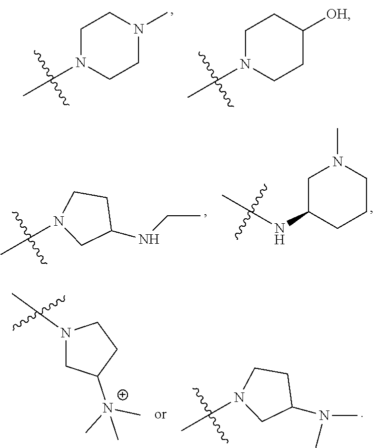

More specifically, the isoxazole derivative may be at least one selected from groups consisted of 5-methyl-N-(2-methyl-5-(3-(4-methylpiperazin-1-yl)-5-(trifluoromethyl)benzamido)phenyl)isoxazole-3-carboxamide(5a);
5-methyl-N-(2-methyl-5-(3-(4-methyl-1H-imidazol-1-yl)-5-(trifluoromethyl)benzamido) phenyl)isoxazole-3-carboxamide(5b);
5-methyl-N-(2-methyl-5-(3-morpholino-5-(trifluoromethyl)benzamido)phenyl)isoxazole-3-carboxamide(5c);
5-methyl-N-(2-methyl-5-(3-(2-methyl-1H-imidazol-1-yl)-5-(trifluoromethyl)benzamido) phenyl)isoxazole-3-carboxamide(5d);
5-methyl-N-(2-methyl-5-(3-(5-methyl-1H-imidazol-1-yl)-5-(trifluoromethyl)benzamido) phenyl)isoxazole-3-carboxamide(5e);
N-(5-([1,1′-biphenyl]-4-carboxamido)-2-methylphenyl)-5-methylisoxazole-3-carboxamide(5f);
5-methyl-N-(2-methyl-5-(3-(2-methyl-1H-imidazol-1-yl)-5-(4-methylpiperazin-1-yl)benzamido) phenyl)isoxazole-3-carboxamide(5g);
5-methyl-N-(2-methyl-5-(3-(2-methyl-1H-imidazol-1-yl)-5-((1-methylpiperidin-4-yl)amino) benzamido)phenyl)isoxazole-3-carboxamide(5h);
5-methyl-N-(2-methyl-5-(3-((1-methylpiperidin-4-yl)amino)-5-(trifluoromethyl)benzamido) phenyl)isoxazole-3-carboxamide, 5i;
N-(5-(3-((4-Ethylpiperazin-1-yl)methyl)-5-(trifluoromethyl)benzamido)-2-methylphenyl)-5-methylisoxazole-3-carboxamide(5j);
N-(5-(3-(4-Hydroxypiperidin-1-yl)-5-(trifluoromethyl)benzamido)-2-methylphenyl)-5-methylisoxazole-3-carboxamide(5k);
(R)-5-Methyl-N-(2-methyl-5-(3-((1-methylpiperidin-3-yl)amino)-5-(trifluoromethyl)benzamido) phenyl)isoxazole-3-carboxamide(5l);
N,N,N-trimethyl-1-(3-((4-methyl-3-(5-methylisoxazole-3-carboxamido)phenyl)carbamoyl)-5-(trifluoromethyl)phenyl)pyrrolidin-3-aminium(5m);
N-(5-(3-(3-(ethylamino)pyrrolidin-1-yl)-5-(trifluoromethyl)benzamido)-2-methylphenyl)-5-methylisoxazole-3-carboxamide(5n);
5-methyl-N-(2-methyl-5-(3-((1-methylpyrrolidin-3-yl)amino)-5-(trifluoromethyl)benzamido)phenyl)isoxazole-3-carboxamide(5o);
N-(5-(3-(3-(dimethylamino)pyrrolidin-1-yl)-5-(trifluoromethyl)benzamido)-2-methylphenyl)-5-methylisoxazole-3-carboxamide(5p);
5-methyl-N-(2-methyl-5-(quinoline-2-carboxamido)phenyl)isoxazole-3-carboxamide(5q);
N-(5-(1H-benzo[d][1,2,3]triazole-6-carboxamido)-2-methylphenyl)-5-methylisoxazole-3-carboxamide(5r);
5-methyl-N-(2-methyl-5-(1-phenyl-5-(trifluoromethyl)-1H-pyrazole-4-carboxamido)phenyl)isoxazole-3-carboxamide(5s);
(N-(5-(1H-indazole-6-carboxamido)-2-methylphenyl)-5-methylisoxazole-3-carboxamide(5t);
N-(5-(3-Chlorobenzamido)-2-methylphenyl)-5-methylisoxazole-3-carboxamide(5u);
5-Methyl-N-(2-methyl-5-(3-((1-methylpiperidin-4-yl)oxy)-5-(trifluoromethyl)benzamido) phenyl)isoxazole-3-carboxamide(5v);
5-methyl-N-(2-methyl-6-((tetrahydro-2H-pyran-4-yl)amino)pyridin-3-yl)isoxazole-3-carboxamide(17b);
5-methyl-N-(5-(pyrrolidin-3-yl)-2′,3′,4′,5′-tetrahydro-[1,1′-biphenyl]-2-yl)isoxazole-3-carboxamide(60a);
5-methyl-N-(5-(piperidin-4-yl)-2′,3′,4′,5′-tetrahydro-[1,1′-biphenyl]-2-yl)isoxazole-3-carboxamide(60b);
N-(5-(azepan-4-yl)-2′,3′,4′,5′-tetrahydro-[1,1′-biphenyl]-2-yl)-5-methylisoxazole-3-carboxamide(60c);
N-(2-(Cyclopent-1-en-1-yl)-4-(piperidin-4-yl)phenyl)-5-methylisoxazole-3-carboxamide(61 b);
5-methyl-N-(4-(piperidin-4-yl)-2-(pyrimidin-5-yl)phenyl)isoxazole-3-carboxamide(62b);
5-Methyl-N-(4′-methyl-5-(piperidin-4-yl)-2′,3′,4′,5′-tetrahydro-[1,1′-biphenyl]-2-yl)isoxazole-3-carboxamide(63b);
N-(4′,4′-dimethyl-5-(piperidin-4-yl)-2′,3′,4′,5′-tetrahydro-[1,1′-biphenyl]-2-yl)-5-methylisoxazole-3-carboxamide(64b);
N-(5-(azepan-4-yl)-4′,4′-dimethyl-2′,3′,4′,5′-tetrahydro-[1,1′-biphenyl]-2-yl)-5-methylisoxazole-3-carboxamide(64c);
N-(2-cyclohexyl-4-(piperidin-4-yl)phenyl)-5-methylisoxazole-3-carboxamide(65b);
N-(2-Cyclopentyl-4-(piperidin-4-yl)phenyl)-5-methylisoxazole-3-carboxamide(66b);
5-methyl-N-(5-(1-methylpiperidin-4-yl)-2′,3′,4′,5′-tetrahydro-[1,1′-biphenyl]-2-yl)isoxazole-3-carboxamide(67b);
5-methyl-N-(5-(1-methylazepan-4-yl)-2′,3′,4′,5′-tetrahydro-[1,1′-biphenyl]-2-yl)isoxazole-3-carboxamide(67c);
N-(4′,4′-dimethyl-5-(1-methylazepan-4-yl)-2′,3′,4′,5′-tetrahydro-[1,1′-biphenyl]-2-yl)-5-methylisoxazole-3-carboxamide(68c);
N-(2-cyclohexyl-4-(1-methylpiperidin-4-yl)phenyl)-5-methylisoxazole-3-carboxamide(69b);
N-(5-(1-ethylpiperidin-4-yl)-2′,3′,4′,5′-tetrahydro-[1,1′-biphenyl]-2-yl)-5-methylisoxazole-3-carboxamide(70b);
N-(2-(Cyclopent-1-en-1-yl)-4-(1-ethylpiperidin-4-yl)phenyl)-5-methylisoxazole-3-carboxamide(71 b);
N-(5-(1-Ethylpiperidin-4-yl)-4′-methyl-2′,3′,4′,5′-tetrahydro-[1,1′-biphenyl]-2-yl)-5-methylisoxazole-3-carboxamide(72b);
N-(5-(1-ethylpiperidin-4-yl)-4′,4′-dimethyl-2′,3′,4′,5′-tetrahydro-[1,1′-biphenyl]-2-yl)-5-methylisoxazole-3-carboxamide(73b);
N-(2-cyclohexyl-4-(1-ethylpiperidin-4-yl)phenyl)-5-methylisoxazole-3-carboxamide(74b);
N-(2-Cyclopentyl-4-(1-ethylpiperidin-4-yl)phenyl)-5-methylisoxazole-3-carboxamide(75b);
5-methyl-N-(5-(1-(3-methylbut-2-en-1-yl)piperidin-4-yl)-2′,3′,4′,5′-tetrahydro-[1,1′-biphenyl]-2-yl)isoxazole-3-carboxamide(76b);
N-(5-(1-(cyclopropylmethyl)piperidin-4-yl)-4′-methyl-2′,3′,4′,5′-tetrahydro-[1,1′-biphenyl]-2-yl)-5-methylisoxazole-3-carboxamide(77b);
N-(5-(1-(cyclopropylmethyl)piperidin-4-yl)-4′,4′-dimethyl-2′,3′,4′,5′-tetrahydro-[1,1′-biphenyl]-2-yl)-5-methylisoxazole-3-carboxamide(78b);
1,1-dimethyl-4-(6-(5-methylisoxazole-3-carboxamido)-2′,3′,4′,5′-tetrahydro-[1,1′-biphenyl]-3-yl)piperidin-1-ium(79b);
1,1-dimethyl-4-(6-(5-methylisoxazole-3-carboxamido)-2′,3′,4′,5′-tetrahydro-[1,1′-biphenyl]-3-yl)azepan-1-ium(79c);
4-(4′,4′-dimethyl-6-(5-methylisoxazole-3-carboxamido)-2′,3′,4′,5′-tetrahydro-[1,1′-biphenyl]-3-yl)-1,1-dimethylazepan-1-ium(80c);
4-(3-cyclohexyl-4-(5-methylisoxazole-3-carboxamido)phenyl)-1,1-dimethylpiperidin-1-ium(81 b);
1,1-diethyl-4-(6-(5-methylisoxazole-3-carboxamido)-2′,3′,4′,5′-tetrahydro-[1,1′-biphenyl]-3-yl)piperidin-1-ium(82b);
N-(5-(1-acetylpiperidin-4-yl)-2′,3′,4′,5′-tetrahydro-[1,1′-biphenyl]-2-yl)-5-methylisoxazole-3-carboxamide(83b);
N-(5-(1-(dimethylglycyl)piperidin-4-yl)-2′,3′,4′,5′-tetrahydro-[1,1′-biphenyl]-2-yl)-5-methylisoxazole-3-carboxamide(84b);
5-methyl-N-(5-(2,2,6,6-tetramethylpiperidin-4-yl)-2′,3′,4′,5′-tetrahydro-[1,1′-biphenyl]-2-yl)isoxazole-3-carboxamide(92a);
5-methyl-N-(4′-methyl-5-(2,2,6,6-tetramethylpiperidin-4-yl)-2′,3′,4′,5′-tetrahydro-[1,1′-biphenyl]-2-yl)isoxazole-3-carboxamide(92b);
N-(4′,4′-dimethyl-5-(2,2,6,6-tetramethylpiperidin-4-yl)-2′,3′,4′,5′-tetrahydro-[1,1′-biphenyl]-2-yl)-5-methylisoxazole-3-carboxamide, 92c; and N-(2-(cyclohept-1-en-1-yl)-4-(2,2,6,6-tetramethylpiperidin-4-yl)phenyl)-5-methylisoxazole-3-carboxamide(92d).

In addition, the present invention can provide a method for preventing, treating, and/or diagnosing diseases associated with CSF-1R, comprising a step of administering the isoxazole derivative or a pharmaceutically acceptable salt thereof to an individual.

As used herein, the term “prevention” refers to all activities of inhibiting or delaying the occurrence, spread, or recurrence of a CSF-1R-related disease by administering the composition of the present invention, and the term “treatment” refers to all activities of improving or beneficially changing the symptoms of the disease by administering the composition of the present invention.

As used herein, the term “pharmaceutical composition” refers to a composition prepared for the purpose of preventing or treating a disease and may be formulated in various forms according to a general method and used. For example, it may be formulated into oral formulations such as powders, granules, tablets, capsules, suspensions, emulsions, syrups, and the like, and may be formulated into formulations for external application, suppositories, and sterile injectable solutions.

In the present invention, “including as an active ingredient” means that the ingredient is included in an amount necessary or sufficient to realize a desired biological effect, and in actual application, the determination of the amount included as an active ingredient is an amount for treating a subject disease, and may be determined in consideration of matters that do not cause other toxicity, and may vary depending on various factors such as, for example, a disease or condition to be treated, a form of a composition to be administered, a size of a subject, a severity of a disease or condition, and the like, and a person having ordinary skill in the art to which the present invention pertains may empirically determine an effective amount of an individual composition without undue experimentation.

In addition, the pharmaceutical composition of the present invention may further include one or more pharmaceutically acceptable carriers in addition to the above-described active ingredients according to each formulation.

The pharmaceutically acceptable carrier may be a mixture of saline, sterile water, Ringer's solution, buffered saline, dextrose solution, maltodextrin solution, glycerol, ethanol, and one or more of these components, and may further include other common additives such as an antioxidant, a buffer, a bacteriostatic agent, and the like, if necessary. In addition, a diluent, a dispersant, a surfactant, a binder, and a lubricant may be additionally added to the formulation for injection such as an aqueous solution, a suspension, an emulsion, and the like, and the formulation may be made into pills, capsules, granules, or tablets. Furthermore, the pharmaceutical composition may be preferably formulated according to each disease or ingredient by appropriate methods in the art or by using methods disclosed in Remington's Pharmaceutical Science (Mack Publishing Company, Easton PA).

In the present invention, the term “pharmaceutically acceptable salt” refers to a formulation of a compound that does not cause serious stimulation to an organism to which the compound is administered and does not impair biological activity and physical properties of the compound. The pharmaceutically acceptable salt may be obtained by reacting the compound of the present invention with an inorganic acid such as hydrochloric acid, bromic acid, sulfuric acid, nitric acid, phosphoric acid, or the like, a sulfonic acid such as methanesulfonic acid, ethanesulfonic acid, p-toluenesulfonic acid, or the like, or an organic carbonic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, salicylic acid, or the like. In addition, the compound of the present invention may be obtained by reacting the compound with a base to form an alkali metal salt such as ammonium salt, sodium or potassium salt, a salt such as alkaline earth metal salt such as calcium or magnesium salt, a salt of organic bases such as dicyclohexylamine, N-methyl-D-glucamine, tris(hydroxymethyl)methylamine, and an amino acid salt such as arginine, lysine, and the like.

In addition, the isoxazole derivative or a pharmaceutically acceptable salt thereof may include all salts, hydrates, and solvates that can be prepared by a conventional method, as well as pharmaceutically acceptable salts thereof.

The composition of the present invention may be orally or parenterally administered in a pharmaceutically effective amount according to a desired method, and the term “pharmaceutically effective amount” as used herein refers to an amount sufficient to treat a disease at a reasonable benefit/risk ratio applicable to medical treatment and not causing side effects, and an effective dose level may be determined according to factors including a patient's health condition, severity, drug activity, drug sensitivity, administration method, administration time, administration route and excretion rate, treatment period, combination or co-used drug, and other factors well known in the medical field.

Preferably, the CSF-1R-related diseases may include, but are not limited to, preferably, degenerative brain disease such as Alzheimer's disease, Parkinson's disease, Huntington's disease, prosencephaly, microcephaly, cerebral palsy, congenital hydrocephalus, Wilson disease, dementias, multi infarct dementia, Frontotemporal dementia, pseudo-dementia, Motor neuron diseases, spinocerebellar ataxia, spinal muscular atrophy.

In the present invention, the term “individual” is not limited as long as it is a mammal, such as livestock, a human, etc., which requires prevention, treatment, and/or diagnosis of a CSF-1R-related disease but may preferably be a human.

The pharmaceutical composition of the present invention may be formulated in various forms for administration to a subject, and a representative formulation for parenteral administration is an injectable formulation, preferably an isotonic aqueous solution or suspension. Injectable formulations can be prepared according to techniques known in the art using suitable dispersants or wetting agents and suspending agents. For example, each component may be dissolved in a saline solution or a buffer solution to be formulated for injection. Formulations for oral administration may also include, for example, ingestible tablets, buccal tablets, troches, capsules, elixirs, suspensions, syrups and wafers, and these formulations may include diluents (e.g., lactose, dextrose, sucrose, mannitol, sorbitol, cellulose and/or glycine) and lubricants (e.g., silica, talc, stearic acid and magnesium or calcium salts thereof and/or polyethylene glycol) in addition to the active ingredient. The tablet may include a binder such as magnesium aluminum silicate, starch paste, gelatin, tragacanth, methylcellulose, sodium carboxymethylcellulose and/or polyvinylpyrrolidine, and in some cases, may further include a disintegrating agent such as starch, agar, alginic acid or a sodium salt thereof, an absorbent, a colorant, a flavoring agent and/or a sweetening agent. The formulation may be prepared by a conventional mixing, granulating, or coating method.

In addition, the pharmaceutical composition of the present invention may further comprise an auxiliary agent such as a preservative, a hydrating agent, an emulsifying accelerator, a salt or a buffer for controlling osmotic pressure, and other therapeutically useful substances, and may be formulated according to a conventional method.

The pharmaceutical composition according to the present invention may be administered via various routes including oral, transdermal, subcutaneous, intravenous or intramuscular routes, and the dose of the active ingredient may be appropriately selected according to various factors such as the route of administration, the age, sex and weight of the patient, and the severity of the patient. In addition, the composition of the present invention may also be administered in parallel with known compounds capable of improving a desired effect.

The route of administration of the pharmaceutical composition according to the present invention may be orally or parenterally administered to humans and animals such as intravenous, subcutaneous, intranasal, or intraperitoneal administration. Oral administration also includes sublingual application. Parenteral administration includes injection and drop-off methods such as subcutaneous injection, intramuscular injection and intravenous injection.

In the pharmaceutical composition of the present invention, the total effective dose of the isoxazole derivative or pharmaceutically acceptable salt thereof according to the present invention may be administered to a patient in a single dose and may be administered by a fractionated treatment protocol in which multiple doses are administered for a long time. The pharmaceutical composition of the present invention may vary the content of the active ingredient depending on the degree of disease, but may be repeatedly administered several times a day in an effective dose of 100 μg to 3,000 mg when administered once based on an adult. However, the concentration of the isoxazole derivative or a pharmaceutically acceptable salt thereof may be determined as an effective dose to a patient in consideration of various factors such as the age, weight, health condition, sex, severity of disease, diet, excretion rate, etc., of the patient, as well as the administration route and the number of treatments.

In addition, the pharmaceutical composition according to the present invention is not particularly limited to the formulation, administration route, and administration method thereof as long as it shows the effect of the present invention, and the pharmaceutical composition according to the present invention may further include a known therapeutic agent for degenerative diseases in addition to the isoxazole derivative or a pharmaceutically acceptable salt thereof as an active ingredient, and may be used in combination with other known treatments for treating these diseases.

The present invention can apply various transformations and have various embodiments, and specific embodiments will be illustrated in the drawings and described in detail in the detailed description. However, this is not intended to limit the present invention to the specific embodiments, and it should be understood that the present invention includes all modifications, equivalents, and replacements included within the spirit and technical scope of the present invention. In describing the present invention, when it is determined that the detailed description of the related known technology may obscure the gist of the present invention, the detailed description thereof will be omitted.

### DETAILED DESCRIPTION OF THE INVENTION

### Embodiment

### <Synthesis>

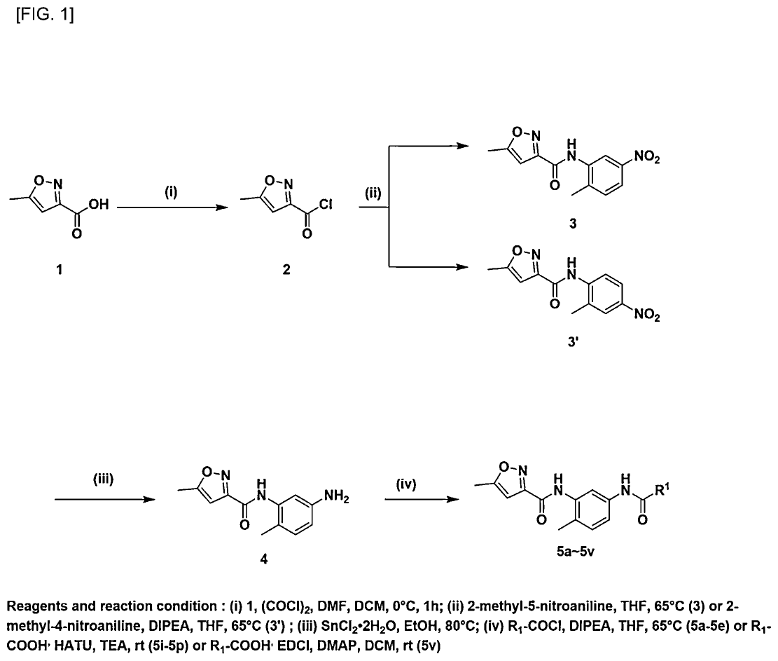

1. Method for synthesizing 5-methylisoxazole-3-carboxamide derivatives (Scheme 1, FIG. 1)

### 5-methylisoxazole-3-carbonyl chloride (2)

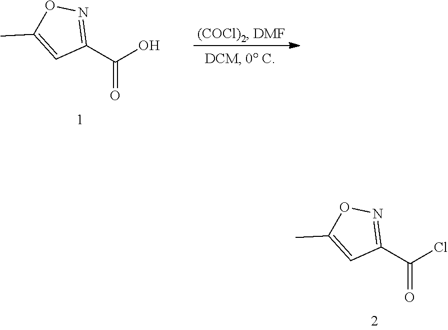

5-methylisoxazole-3-carboxylic acid (200 mg, 1.53 mmol) was dissolved in Dichloromethane (3.06 mL), Dimethylformamide (0.095 mL, 1.224 mmol) and Oxalyl chloride (0.162 mL, 1.884 mmol) were added dropwise at 0° C., and the mixture was stirred for 1 hour. After the completion of the reaction was confirmed, the process of diluting with a small amount of dichloromethane and concentrating was repeated three times to remove oxalyl chloride.

### 5-methyl-N-(2-methyl-5-nitrophenyl)isoxazole-3-carboxamide (3)

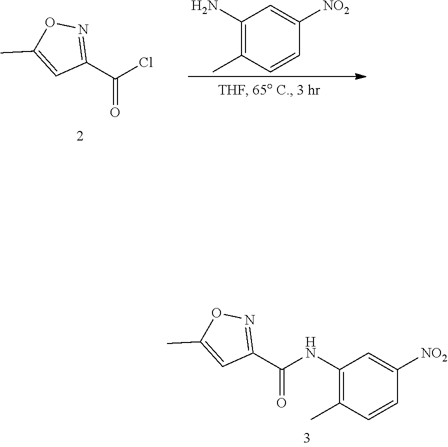

2-methyl-5-nitroaniline (436 mg, 2.87 mmol) was dissolved in THE (10 mL), and then Compound 2 was dissolved in THE (4.6 mL) to give dropwise. After 3 hours, upon completion of the reaction, the reaction mixture was diluted with EA and washed with NaHCO₃ and Brine. The moisture of the EA layer was removed by using Na2SO4 and distilled under reduced pressure to obtain Compound 3 (570.2 mg, 2.18 mmol, 76%).

1H NMR (400 MHz, DMSO-d6) δ 10.43 (s, 1H), 8.33 (d, J=2.4 Hz, 1H), 8.06 (dd, J=8.4, 2.5 Hz, 1H), 7.58 (d, J=8.5 Hz, 1H), 6.70 (d, J=0.9 Hz, 1H), 2.52 (d, J=0.8 Hz, 3H), 2.37 (s, 3H).

HRMS (ESI+) calculated for C12H12N3O4 [M+H]+: 262.0822, found 262.5355

### 5-methyl-N-(2-methyl-4-nitrophenyl)isoxazole-3-carboxamide (3′)

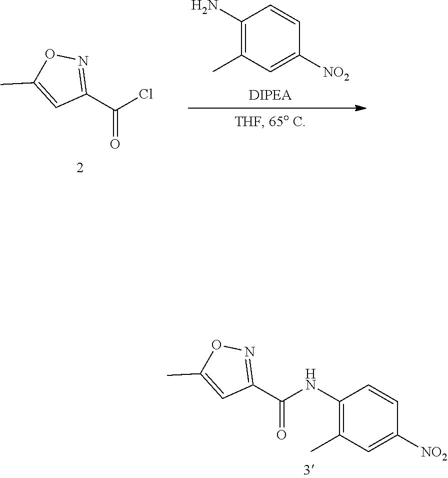

2-methyl-4-nitroaniline (211.5 mg, 1.39 mmol) was dissolved in Tetrahydrofuran (7 mL), 2 (222 mg, 1.53 mmol) was added thereto, DIPEA (0.24 mL, 1.53 mmol) was added dropwise thereto, and the resulting mixture was stirred for 1 hour. After completion of the reaction, the reaction product was extracted with Ethyl acetate, and washed with water and brine. After drying with Anhydrous Na2SO4, the mixture was concentrated. Compound 3′ (347 mg) was purified by column chromatography. 96%) was obtained.

1H NMR (400 MHz, CDCl3) δ 8.73 (s, 1H), 8.50 (d, J=8.9 Hz, 1H), 8.16 (dd, J=8.9, 2.7 Hz, 1H), 8.14-8.12 (m, 1H), 6.56 (d, J=0.9 Hz, 1H), 2.54 (d, J=0.9 Hz, 3H), 2.47 (s, 3H).

HRMS(ESI+) calculated for C12H12N3O4 [M+H]+: 262.0822, found 262.3193.

### N-(5-amino-2-methylphenyl)-5-methylisoxazole-3-carboxamide (4)

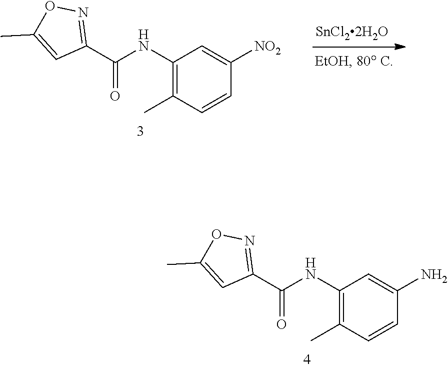 [SMILES: Cc1cc(C(=O)Nc2cc([N+](=O)[O-])ccc2C)no1 ; Cc1cc(C(=O)Nc2cc(N)ccc2C)no1]

Compound 3 (500 mg, 0.19 mmol) obtained above, and Tin(II)chloride dehydrate (214 mg, 0.95 mmol) were added thereto and dissolved in Ethanol (0.38 mL), followed by stirring at 80° C. for 1 hour. After the completion of the reaction was confirmed, the reaction mixture was extracted with Ethyl acetate, and the organic layer was washed with NaHCO₃ and brine. Moisture was removed with Anhydrous Na2SO4, and the solvent was distilled under reduced pressure to obtain Compound 4 (42.4 mg, 96%).

1H NMR (400 MHz, DMSO-d6) δ 9.81 (s, 1H), 6.88 (d, J=8.1 Hz, 1H), 6.63 (d, J=2.2 Hz, 1H), 6.62 (s, 1H), 6.45-6.35 (m, 1H), 4.93 (d, J=8.7 Hz, 2H), 2.49 (s, 3H), 2.03 (s, 3H).

HRMS (ESI+) calculated for C12H14N3O2 [M+H]+: 232.1081, found 232.2427.

### N-(4-amino-2-methylphenyl)-5-methylisoxazole-3-carboxamide (4′)

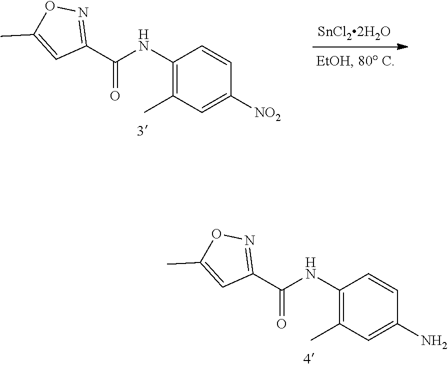 [SMILES: Cc1cc(C(=O)Nc2ccc([N+](=O)[O-])cc2C)no1 ; Cc1cc(C(=O)Nc2ccc(N)cc2C)no1]

The reaction mixture was synthesized in the same manner as above, and obtained Compound 4′ (87.5 mg, 98%) was obtained by the same method as above without further purification.

1H NMR (400 MHz, CDCl3) δ 8.20 (s, 1H), 7.59-7.51 (m, 1H), 6.61-6.52 (m, 2H), 6.51 (d, J=0.8 Hz, 1H), 3.36 (dd, J=139.8, 53.5 Hz, 2H), 2.50 (d, J=0.6 Hz, 3H), 2.24 (s, 3H).

HRMS(ESI+) calculated for C12H14N3O2 [M+H]+: 232.1081, found 232.3140

### 3-(4-methylpiperazin-1-yl)-5-(trifluoromethyl)benzoyl chloride

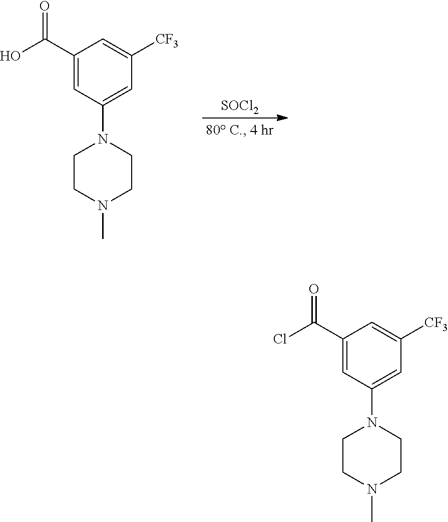 [SMILES: CN1CCN(c2cc(C(=O)O)cc(C(F)(F)F)c2)CC1 ; "SOCl2" ; CN1CCN(c2cc(C(=O)Cl)cc(C(F)(F)F)c2)CC1]

3-(4-methylpiperazin-1-yl)-5-(trifluoromethyl)benzoic acid (405 mg, 1.40 mmol) and SOCl2 (1.53 mL) were added thereto, and the mixture was stirred at 80° C. for 4 hours. After completion of the reaction, MC was added to the reaction mixture and distilled under reduced pressure to remove the acid. The reaction was terminated three times through this process.

### 5-methyl-N-(2-methyl-5-(3-(4-methylpiperazin-1-yl)-5-(trifluoromethyl)benzamido)phenyl)isoxazole-3-carboxamide (5a)

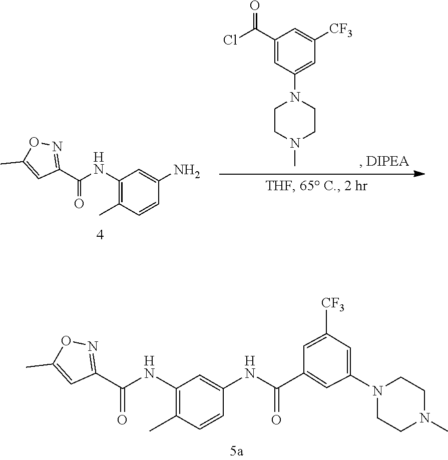

Compound 4 (171 mg, 0.739 mmol) was dissolved in THE (4 mL), 3-(4-methylpiperazin-1-yl)-5-(trifluoromethyl)benzoyl chloride was dissolved in THE (3.39 mL) to give dropwise, and DIPEA (125f) was added thereto, and the resulting mixture was stirred at 65° C. for 2 hours. After completion of the reaction, the reaction mixture was diluted with EA and washed with NaHCO₃, Brine. The moisture of the EA layer was removed by Na2SO4 and distilled under reduced pressure. Column chromatography (developing solvent MC:MeOH=40:1) was performed to obtain compound 5a (54.8 mg, 0.109 mmol, 14.7%).

1H NMR (400 MHz, DMSO-d6) δ 10.34 (s, 1H), 10.17 (s, 1H), 7.81 (d, J=2.1 Hz, 1H), 7.71 (s, 1H), 7.64-7.60 (m, 2H), 7.36 (s, 1H), 7.26 (d, J=8.6 Hz, 1H), 6.66 (s, 1H), 3.30 (s, 3H), 2.47 (d, J=5.2 Hz, 8H), 2.24 (s, 3H), 2.20 (s, 3H).

HRMS (ESI+) calculated for C25H27F3N5O3 [M+H]+: 502.2061, found 502.5013

### 3-(4-methyl-1H-imidazol-1-yl)-5-(trifluoromethyl)benzoyl chloride

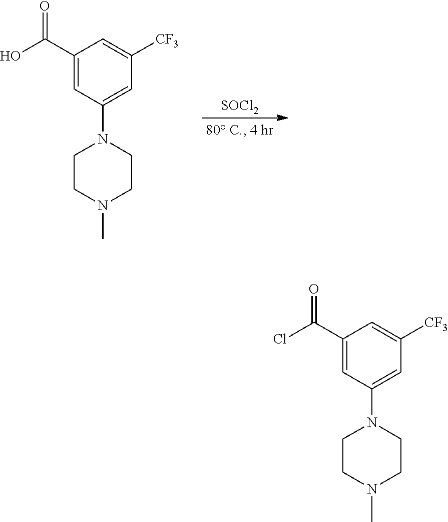 [SMILES: CN1CCN(c2cc(C(=O)O)cc(C(F)(F)F)c2)CC1 ; "SOCl2" ; CN1CCN(c2cc(C(=O)Cl)cc(C(F)(F)F)c2)CC1]

3-morpholino-5-(trifluoromethyl)benzoic acid (175.4 mg, 0.6492 mmol) was dissolved in Thionyl chloride (1 mL) and stirred at 80° C. After confirming the completion of the reaction, it was diluted with a small amount of Dichloromethane, concentrated three times, and obtained by removing thionyl chloride.

### 5-methyl-N-(2-methyl-5-(3-(4-methyl-1H-imidazol-1-yl)-5-(trifluoromethyl)benzamido) phenyl)isoxazole-3-carboxamide (5b)

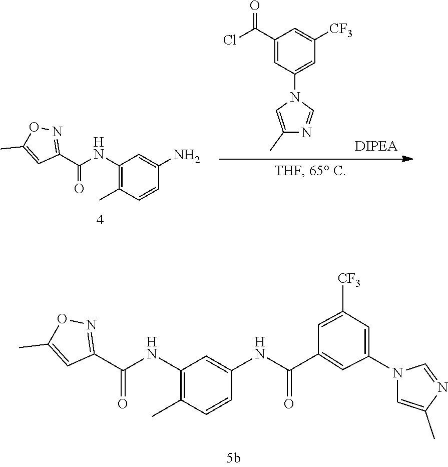 [SMILES: Cc1cc(C(=O)Nc2cc(N)ccc2C)no1 ; Cc1cn(-c2cc(C(=O)Cl)cc(C(F)(F)F)c2)cn1 ; Cc1cn(-c2cc(C(=O)Nc3ccc(C)c(NC(=O)c4cc(C)on4)c3)cc(C(F)(F)F)c2)cn1]

The reaction mixture was synthesized in the same manner as described above, and separated and purified through column chromatography to obtain 57 mg (0.118 mmol, 24.2%) of Compound 5b.

1H NMR (400 MHz, MeOD) δ 8.40 (s, 1H), 8.27 (d, J=1.4 Hz, 1H), 8.24 (s, 1H), 8.11 (s, 1H), 7.97 (d, J=2.2 Hz, 1H), 7.61 (dd, J=8.3, 2.2 Hz, 1H), 7.51 (s, 1H), 7.31 (d, J=8.5 Hz, 1H), 6.57 (d, J=0.8 Hz, 1H), 2.53 (d, J=0.8 Hz, 3H), 2.31 (s, 3H), 2.28 (d, J=1.0 Hz, 3H).

HRMS (ESI+) calculated for C24H21F3N5O3 [M+H]+: 484.1591, found 484.4957.

### 3-morpholino-5-(trifluoromethyl)benzoyl chloride

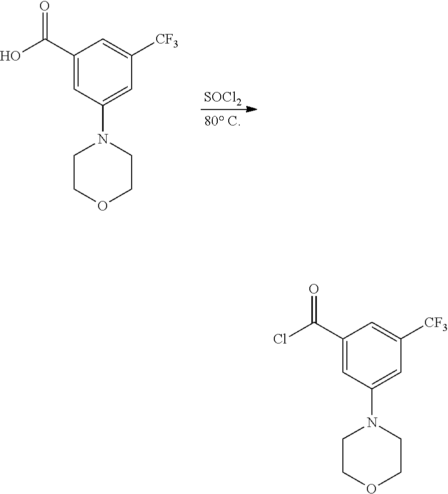

3-(4-methyl-1H-imidazol-1-yl)-5-(trifluoromethyl)benzoic acid (50 mg, 0.18 mmol) was dissolved in Thionyl chloride (0.25 mL) and stirred at 80° C. After confirming the completion of the reaction, it was diluted with a small amount of Dichloromethane, concentrated three times, and obtained by removing thionyl chloride.

### 5-methyl-N-(2-methyl-5-(3-morpholino-5-(trifluoromethyl)benzamido)phenyl)isoxazole-3-carboxamide (5c)

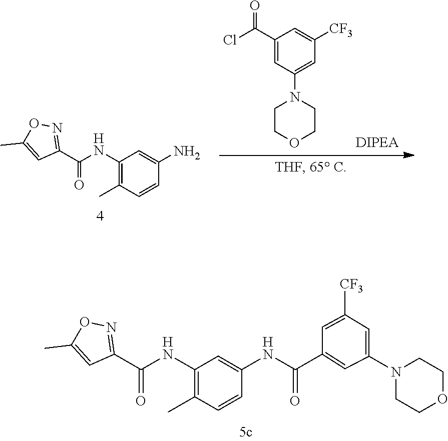 [SMILES: Cc1cc(C(=O)Nc2cc(N)ccc2C)no1 ; O=C(Cl)c1cc(N2CCOCC2)cc(C(F)(F)F)c1 ; Cc1cc(C(=O)Nc2cc(NC(=O)c3cc(N4CCOCC4)cc(C(F)(F)F)c3)ccc2C)no1]

The reaction mixture was synthesized in the same manner as above, and separated and purified through column chromatography to obtain 42 mg (0.086 mmol, 48%) of Compound 5c.

1H NMR (400 MHz, CDCl3) δ 8.50 (s, 1H), 8.21 (d, J=2.2 Hz, 1H), 8.05 (s, 1H), 7.75 (dd, J=8.3, 2.2 Hz, 1H), 7.73 (s, 1H), 7.55 (s, 1H), 7.32 (s, 1H), 7.23 (d, J=8.4 Hz, 1H), 6.52 (d, J=0.9 Hz, 1H), 3.97-3.85 (m, 4H), 3.40-3.22 (m, 4H), 2.53 (d, J=0.8 Hz, 3H), 2.34 (s, 3H).

HRMS (ESI+) calculated for C24H24F3N4O4 [M+H]+: 489.1744, found 489.4292.

### 3-(2-methyl-1H-imidazol-1-yl)-5-(trifluoromethyl)benzoyl chloride

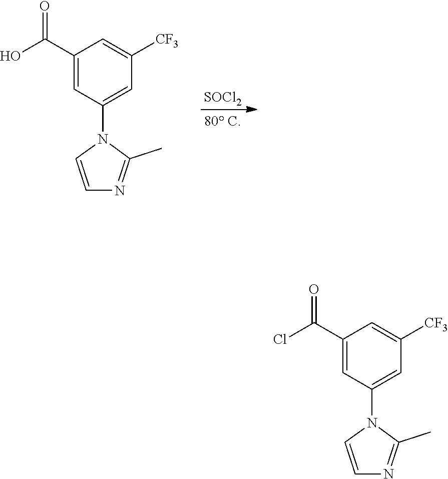

3-(2-methyl-1H-imidazol-1-yl)-5-(trifluoromethyl)benzoic acid (49 mg, 0.18 mmol) was dissolved in Thionyl chloride (0.25 mL) and stirred at 80° C. After confirming the completion of the reaction, it was diluted with a small amount of Dichloromethane, concentrated three times, and obtained by removing thionyl chloride.

### 5-methyl-N-(2-methyl-5-(3-(2-methyl-1H-imidazol-1-yl)-5-(trifluoromethyl)benzamido) phenyl)isoxazole-3-carboxamide (5d)

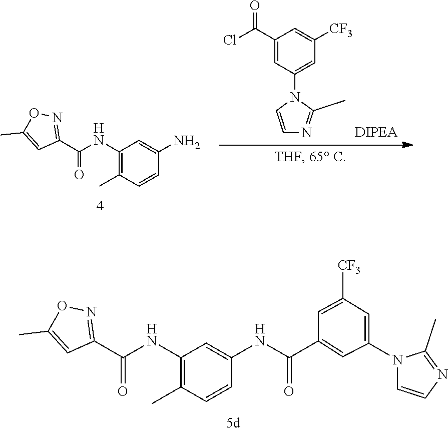 [SMILES: Cc1cc(C(=O)Nc2cc(N)ccc2C)no1 ; Cc1nccn1-c1cc(C(=O)Cl)cc(C(F)(F)F)c1 ; Cc1cc(C(=O)Nc2cc(NC(=O)c3cc(-n4ccnc4C)cc(C(F)(F)F)c3)ccc2C)no1]

The reaction mixture was synthesized in the same manner as above, and separated and purified through column chromatography to obtain 30 mg (0.062 mmol, 34%) of Compound 5d.

1H NMR (400 MHz, MeOD) δ 8.39 (s, 1H), 8.26 (s, 1H), 8.02 (s, 1H), 7.96 (d, J=2.1 Hz, 1H), 7.60 (dd, J=8.3, 2.2 Hz, 1H), 7.35 (d, J=1.5 Hz, 1H), 7.31 (d, J=8.4 Hz, 1H), 7.04 (d, J=1.5 Hz, 1H), 6.56 (d, J=0.8 Hz, 1H), 2.52 (d, J=0.7 Hz, 3H), 2.41 (s, 3H), 2.30 (d, J=4.1 Hz, 3H).

HRMS (ESI+) calculated for C24H21F3N5O3 [M+H]+: 484.1591, found 484.4237.

### 3-(5-methyl-1H-imidazol-1-yl)-5-(trifluoromethyl)benzoyl chloride

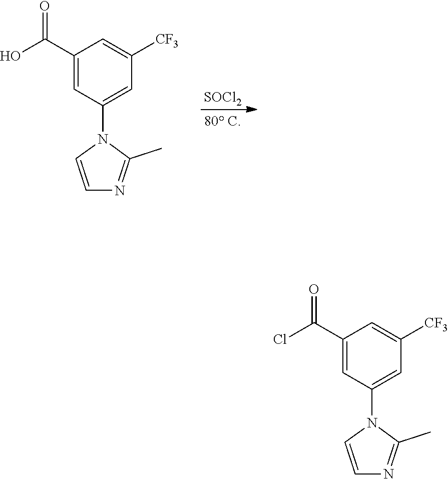

3-(5-methyl-1H-imidazol-1-yl)-5-(trifluoromethyl)benzoic acid (51 mg, 0.189 mmol) was dissolved in Thionyl chloride (0.26 mL) and stirred at 80° C. After confirming the completion of the reaction, it was diluted with a small amount of Dichloromethane, concentrated three times, and obtained by removing thionyl chloride.

### 5-methyl-N-(2-methyl-5-(3-(5-methyl-1H-imidazol-1-yl)-5-(trifluoromethyl)benzamido) phenyl)isoxazole-3-carboxamide (5e)

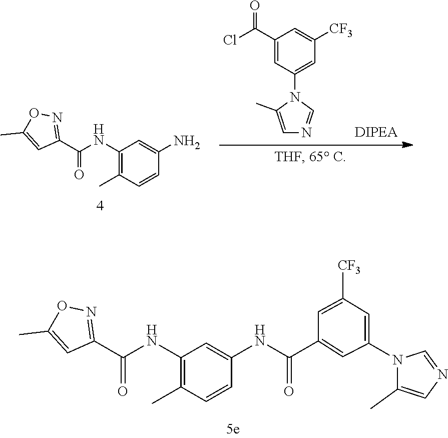 [SMILES: Cc1cc(C(=O)Nc2cc(N)ccc2C)no1 ; Cc1cncn1-c1cc(C(=O)Cl)cc(C(F)(F)F)c1 ; Cc1cc(C(=O)Nc2cc(NC(=O)c3cc(-n4cncc4C)cc(C(F)(F)F)c3)ccc2C)no1]

The reaction mixture was synthesized in the same manner as above, and separated and purified through column chromatography to obtain Compound 5e (38 mg, 0.786 mmol, 50%).

1H NMR (400 MHz, MeOD) δ 8.41 (s, 1H), 8.27 (s, 1H), 8.01 (s, 1H), 7.96 (d, J=2.1 Hz, 1H), 7.90 (s, 1H), 7.60 (dd, J=8.3, 2.2 Hz, 1H), 7.31 (d, J=8.3 Hz, 1H), 6.93 (s, 1H), 6.56 (d, J=0.8 Hz, 1H), 2.53 (d, J=0.7 Hz, 3H), 2.31 (s, 3H), 2.26 (d, J=0.8 Hz, 3H).

HRMS (ESI+) calculated for C24H21F3N5O3 [M+H]+: 484.1591, found 484.4237.

### [1,1′-biphenyl]-4-carbonyl chloride

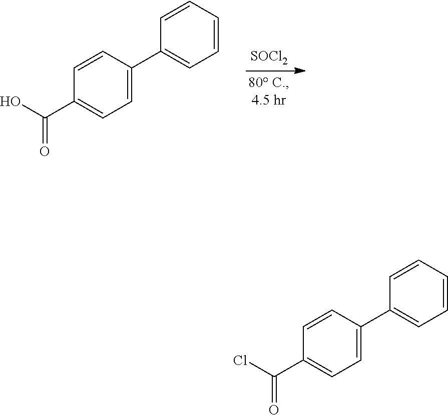

[1,1′-biphenyl]-4-carboxylic acid (10.3 mg, 0.052 mmol) and SOCl2 (75.3 f) were added thereto, and the mixture was stirred at 80° C. for 4.5 hours. After completion of the reaction, MC was added to the reaction mixture and distilled under reduced pressure to remove the acid. The reaction was terminated three times through this process.

### N-(5-([1,1′-biphenyl]-4-carboxamido)-2-methylphenyl)-5-methylisoxazole-3-carboxamide (5f)

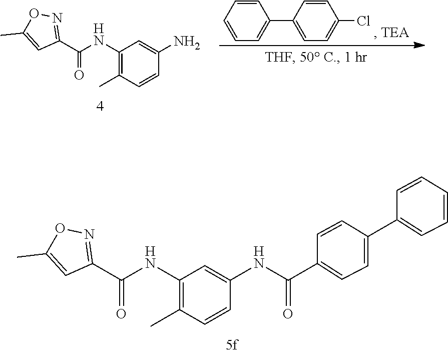 [SMILES: Cc1cc(C(=O)Nc2cc(N)ccc2C)no1 ; Clc1ccc(-c2ccccc2)cc1 ; Cc1cc(C(=O)Nc2cc(NC(=O)c3ccc(-c4ccccc4)cc3)ccc2C)no1]

The reaction mixture was synthesized in the same manner as described above, and distilled under reduced pressure to obtain a concentrated mixture, was diluted by adding ethyl acetate (EA) thereto, and hexane (Hex) was added thereto to precipitate a solid product, and then filtered to obtain Compound 5f (2.4 mg, 0.0058 mmol, 13.5%).

1H NMR (400 MHz, DMSO-d6) δ 10.31 (s, 1H), 10.18 (s, 1H), 8.10-8.04 (m, 2H), 7.89 (d, J=2.0 Hz, 1H), 7.87-7.81 (m, 2H), 7.77 (dd, J=5.2, 3.3 Hz, 2H), 7.64 (dd, J=8.3, 2.2 Hz, 1H), 7.55-7.48 (m, 2H), 7.46-7.40 (m, 1H), 7.25 (d, J=8.5 Hz, 1H), 6.67 (s, 1H), 2.51 (s, 3H), 2.20 (s, 3H).

HRMS (ESI+) calculated for C25H22N3O3 [M+H]+: 412.4685, found 412.4348

### 5-methyl-N-(2-methyl-5-(3-(2-methyl-1H-imidazol-1-yl)-5-(4-methylpiperazin-1-yl)benzamido) phenyl)isoxazole-3-carboxamide (5g)

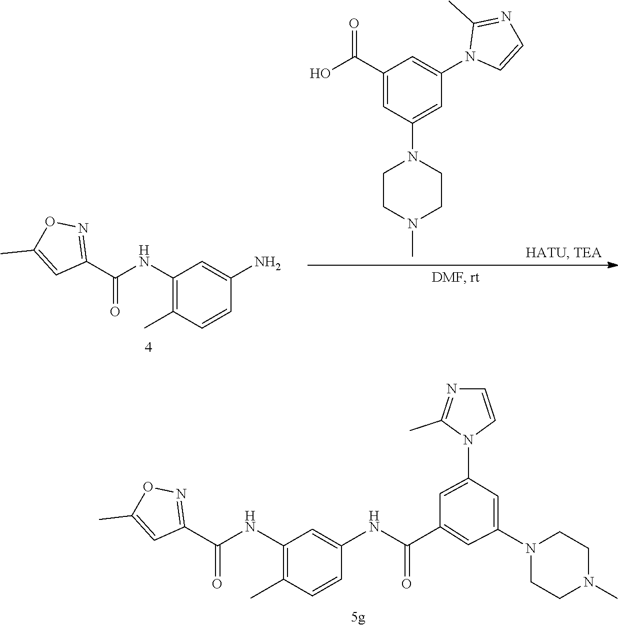

acid (78 mg, 0.26 mmol) of 3-(2-methyl-1H-imidazol-1-yl)-5-(4-methylpiperazin-1-yl)benzoic and HATU (74 mg, 0.195 mmol) were added thereto, dissolved in DMF (1.3 mL), and TEA (36 μL, 0.26 mmol) was added thereto, followed by stirring. Compound 4 (30 mg, 0.13 mmol) obtained above was added thereto and stirred. After confirming the completion of the reaction, the reaction mixture was extracted with Ethyl acetate, and the organic layer was washed with water and brine. Moisture was removed by using Anhydrous Na2SO4, the solvent was concentrated, and then the resulting product was separated and purified by column chromatography to obtain 10.7 mg (0.020 mmol) of Compound 5g (yield: 15%).

1H NMR (400 MHz, Acetone) δ 9.63 (s, 1H), 9.03 (s, 1H), 8.18 (dd, J=6.1, 2.1 Hz, 1H), 7.75-7.72 (m, 1H), 7.63 (d, J=2.1 Hz, 1H), 7.38 (d, J=1.5 Hz, 1H), 7.26 (d, J=8.3 Hz, 1H), 7.18 (d, J=1.3 Hz, 1H), 7.13-7.10 (m, 1H), 6.90 (d, J=1.3 Hz, 1H), 6.59 (s, 1H), 3.39-3.33 (m, 4H), 2.54 (d, J=0.8 Hz, 3H), 2.53-2.50 (m, 4H), 2.33 (s, 3H), 2.31 (s, 3H), 2.27 (s, 3H).

HRMS (ESI+) calculated for C28H32N7O3 [M+H]+: 514.2561, found 514.4929.

### 5-methyl-N-(2-methyl-5-(3-(2-methyl-1H-imidazol-1-yl)-5-((1-methylpiperidin-4-yl)amino) benzamido)phenyl)isoxazole-3-carboxamide (5h)

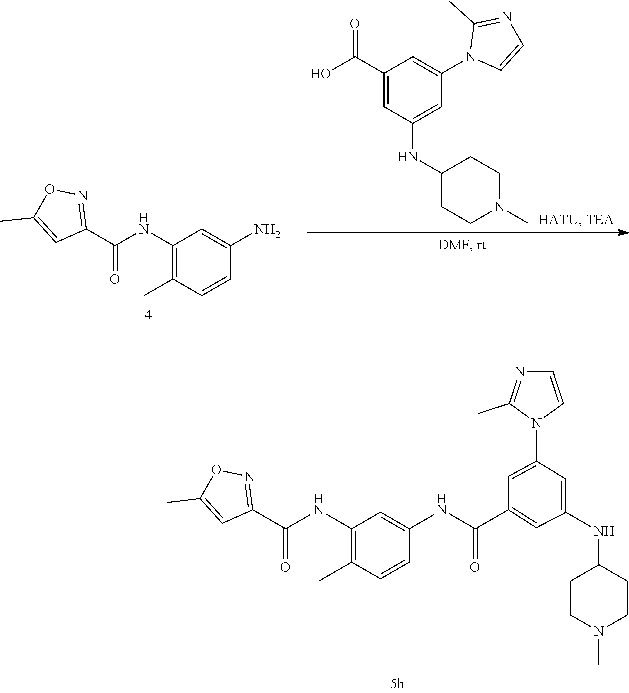 [SMILES: Cc1cc(C(=O)Nc2cc(N)ccc2C)no1 ; Cc1nccn1-c1cc(NC2CCN(C)CC2)cc(C(=O)O)c1 ; Cc1cc(C(=O)Nc2cc(NC(=O)c3cc(NC4CCN(C)CC4)cc(-n4ccnc4C)c3)ccc2C)no1]

The reaction mixture was synthesized in the same manner as above, and separated and purified through column chromatography to obtain Compound 5h (10 mg, 18%).

1H NMR (400 MHz, DMSO-d6) δ 10.17 (s, 1H), 10.14 (s, 1H), 7.82 (d, J=2.0 Hz, 1H), 7.59 (dd, J=8.3, 2.1 Hz, 1H), 7.30 (d, J=1.3 Hz, 1H), 7.23 (d, J=8.6 Hz, 1H), 7.19 (s, 1H), 7.12 (s, 1H), 6.91 (d, J=1.3 Hz, 1H), 6.78 (s, 1H), 6.65 (s, 1H), 6.25 (d, J=7.3 Hz, 1H), 3.47 (s, 1H), 3.03 (s, 2H), 2.50 (s, 3H), 2.44 (s, 3H), 2.32 (s, 3H), 2.19 (s, 3H), 1.99 (s, 2H), 1.56 (s, 2H), 1.24 (s, 2H).

HRMS (ESI+) calculated for C29H34N7O3 [M+H]+:528.2718, found 528.5010

### 5-methyl-N-(2-methyl-5-(3-((1-methylpiperidin-4-yl)amino)-5-(trifluoromethyl)benzamido) phenyl)isoxazole-3-carboxamide (5i)

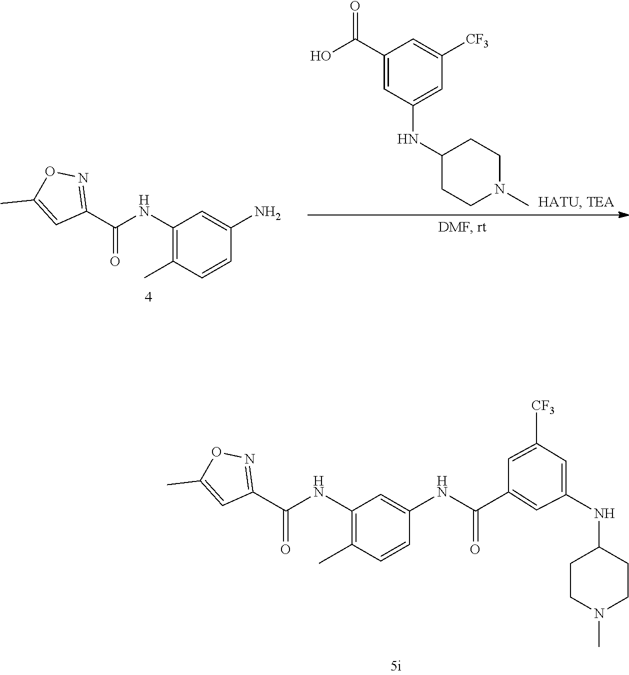 [SMILES: Cc1cc(C(=O)Nc2cc(N)ccc2C)no1 ; CN1CCC(Nc2cc(C(=O)O)cc(C(F)(F)F)c2)CC1 ; Cc1cc(C(=O)Nc2cc(NC(=O)c3cc(NC4CCN(C)CC4)cc(C(F)(F)F)c3)ccc2C)no1]

Compound 5i (21 mg, 0.040 mmol, 40%) was obtained by synthesis in the same manner as in 5g and separation and purification through column chromatography.

1H NMR (400 MHz, DMSO-d6) δ 10.27 (s, 1H), 10.17 (s, 1H), 7.81 (d, J=2.0 Hz, 1H), 7.60 (dd, J=8.3, 2.2 Hz, 1H), 7.33 (d, J=14.3 Hz, 2H), 7.24 (d, J=8.6 Hz, 1H), 7.02 (s, 1H), 6.66 (d, J=0.8 Hz, 1H), 6.29 (d, J=7.9 Hz, 1H), 3.34 (d, J=3.2 Hz, 1H), 2.73 (d, J=11.4 Hz, 2H), 2.51 (s, 3H), 2.19 (d, J=4.4 Hz, 6H), 2.07 (t, J=10.3 Hz, 2H), 1.89 (d, J=11.3 Hz, 2H), 1.42 (dd, J=20.4, 10.1 Hz, 2H).

HRMS (ESI+) calculated for C26H29F3N5O3 [M+H]+:516.2217, found 516.5455.

### N-(5-(3-((4-Ethylpiperazin-1-yl)methyl)-5-(trifluoromethyl)benzamido)-2-methylphenyl)-5-methylisoxazole-3-carboxamide (5j)

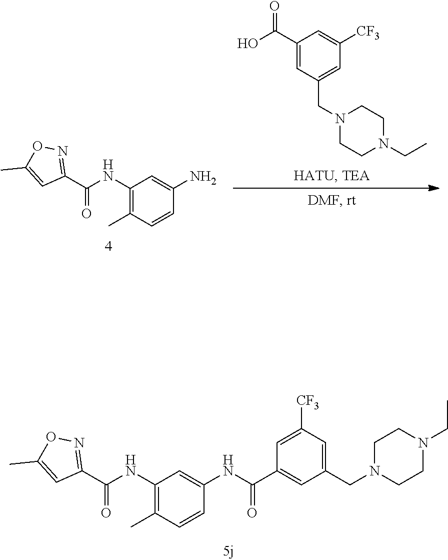 [SMILES: Cc1cc(C(=O)Nc2cc(N)ccc2C)no1 ; CCN1CCN(Cc2cc(C(=O)O)cc(C(F)(F)F)c2)CC1 ; CCN1CCN(Cc2cc(C(=O)Nc3ccc(C)c(NC(=O)c4cc(C)on4)c3)cc(C(F)(F)F)c2)CC1]

Compound 5j (25 mg, 0.048 mmol, 58%) was obtained by performing synthesis in the same manner as in 5g and separation and purification through column chromatography.

1H NMR (400 MHz, MeOD) δ 8.19 (s, 2H), 7.94 (d, J=2.1 Hz, 1H), 7.90 (s, 1H), 7.58 (dd, J=8.3, 2.2 Hz, 1H), 7.31 (d, J=8.5 Hz, 1H), 6.56 (d, J=0.8 Hz, 1H), 3.80 (s, 2H), 3.60-3.44 (m, 2H), 3.20 (d, J=7.3 Hz, 6H), 2.53 (d, J=0.8 Hz, 5H), 2.31 (s, 3H), 1.34 (s, 3H);

HRMS(ESI+) calculated for C27H31F3N5O3 [M+H]+: 530.2061, found 530.8416.

### N-(5-(3-(4-Hydroxypiperidin-1-yl)-5-(trifluoromethyl)benzamido)-2-methylphenyl)-5-methylisoxazole-3-carboxamide (5k)

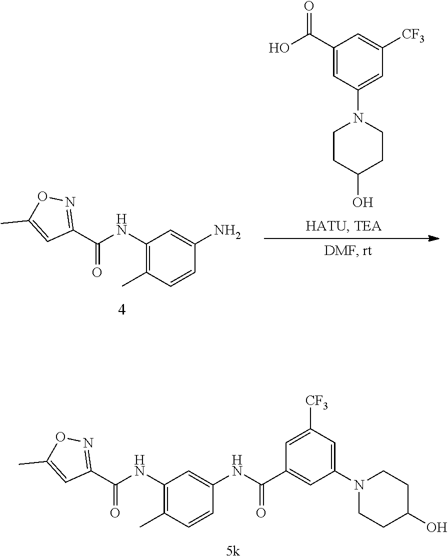 [SMILES: Cc1cc(C(=O)Nc2cc(N)ccc2C)no1 ; O=C(O)c1cc(N2CCC(O)CC2)cc(C(F)(F)F)c1 ; Cc1cc(C(=O)Nc2cc(NC(=O)c3cc(N4CCC(O)CC4)cc(C(F)(F)F)c3)ccc2C)no1]

The reaction mixture was synthesized in the same manner as in the above 5g, and separated and purified through column chromatography to obtain 26 mg (0.053 mmol) of Compound 5k (yield: 81%).

1H NMR (400 MHz, MeOD) δ 7.91 (d, J=2.2 Hz, 1H), 7.71 (s, 1H), 7.60-7.55 (m, 2H), 7.34 (s, 1H), 7.29 (d, J=8.4 Hz, 1H), 6.56 (d, J=0.8 Hz, 1H), 3.86-3.71 (m, 3H), 3.07 (ddd, J=13.0, 10.2, 3.1 Hz, 2H), 2.52 (d, J=0.8 Hz, 3H), 2.30 (s, 3H), 2.04-1.95 (m, 2H), 1.70-1.60 (m, 2H); HRMS(ESI+) calculated for C25H26F3N4O4 [M+H]+: 503.1901, found 503.5456

### (R)-5-Methyl-N-(2-methyl-5-(3-((1-methylpiperidin-3-yl)amino)-5-(trifluoromethyl)benzamido) phenyl)isoxazole-3-carboxamide (5l)

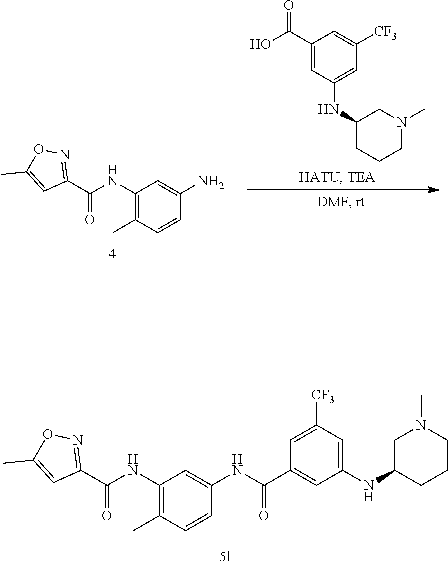 [SMILES: Cc1cc(C(=O)Nc2cc(N)ccc2C)no1 ; CN1CCC[C@@H](Nc2cc(C(=O)O)cc(C(F)(F)F)c2)C1 ; Cc1cc(C(=O)Nc2cc(NC(=O)c3cc(N[C@@H]4CCCN(C)C4)cc(C(F)(F)F)c3)ccc2C)no1]

Compound 51 (0.9 mg, 0.0017 mmol, 2.68%) was obtained by carrying out the synthesis in the same manner as in the above 5g, and separation and purification through column chromatography.

1H NMR (400 MHz, DMSO-d6) δ 10.28 (s, 1H), 10.17 (s, 1H), 7.82 (s, 1H), 7.59 (s, 1H), 7.36 (s, 1H), 7.25 (d, J=8.6 Hz, 1H), 7.06 (s, 1H), 6.66 (s, 1H), 6.25 (d, J=8.5 Hz, 1H), 4.81-4.73 (m, 1H), 2.82-2.77 (m, 1H), 2.20 (s, 3H), 2.17 (d, J=5.3 Hz, 3H), 1.99-1.98 (m, 3H), 1.86-1.80 (m, 2H), 1.74-1.68 (m, 2H), 1.54 (s, 2H), 1.45 (s, 2H).

HRMS(ESI+) calculated for C26H29F3N5O3 [M+H]+: 516.2217, found 516.5815

### N,N,N-trimethyl-1-(3-((4-methyl-3-(5-methylisoxazole-3-carboxamido)phenyl)carbamoyl)-5-(trifluoromethyl)phenyl)pyrrolidin-3-aminium (5m)

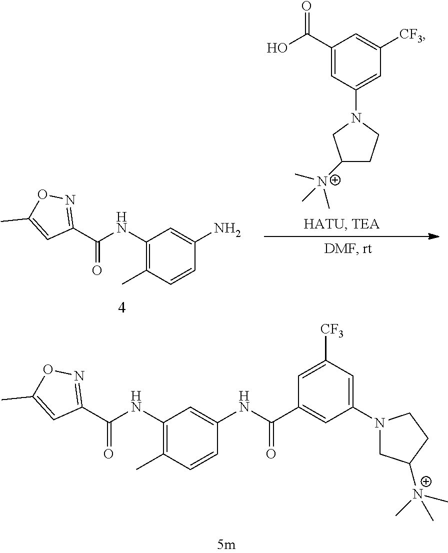

The reaction mixture was synthesized in the same manner as in the above 5g, and separated and purified through column chromatography to obtain 2.5 mg (0.0047 mmol) of Compound 5m (yield: 11%).

1H NMR (400 MHz, DMSO-d6) δ 10.37 (s, 1H), 10.17 (s, 1H), 7.83 (d, J=1.9 Hz, 1H), 7.63 (dd, J=8.3, 2.2 Hz, 1H), 7.59 (s, 1H), 7.45 (s, 1H), 7.26 (d, J=8.6 Hz, 1H), 7.09 (s, 1H), 6.66 (s, 1H), 4.35 (dd, J=14.0, 7.6 Hz, 1H), 3.95-3.88 (m, 1H), 3.73-3.62 (m, 2H), 3.37 (s, 1H), 3.15 (s, 9H), 2.51 (s, 3H), 2.44 (dd, J=15.1, 7.6 Hz, 2H), 2.21 (s, 3H).)

HRMS (ESI+) calculated for C27H31F3N5O3 [M+H]+: 530.2374, found 530.5896

### N-(5-(3-(3-(ethylamino)pyrrolidin-1-yl)-5-(trifluoromethyl)benzamido)-2-methylphenyl)-5-methylisoxazole-3-carboxamide (5n)

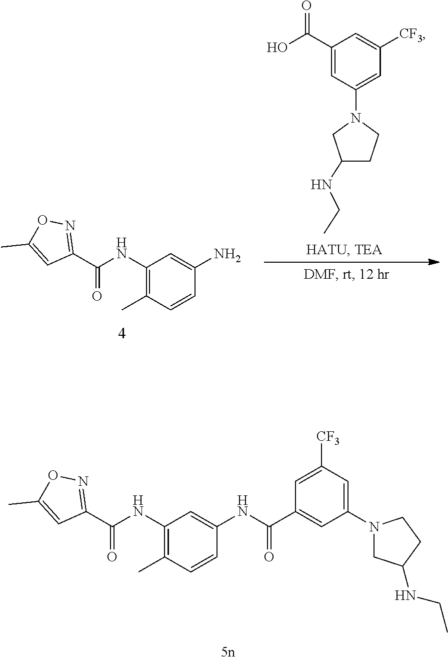 [SMILES: Cc1cc(C(=O)Nc2cc(N)ccc2C)no1 ; CCNC1CCN(c2cc(C(=O)O)cc(C(F)(F)F)c2)C1 ; CCNC1CCN(c2cc(C(=O)Nc3ccc(C)c(NC(=O)c4cc(C)on4)c3)cc(C(F)(F)F)c2)C1]

The reaction mixture was synthesized in the same manner as in the above 5g, and separated and purified through column chromatography to obtain 9.2 mg (0.0179 mmol, 11.2%) of Compound 5n.

1H NMR (400 MHz, DMSO-d6) δ 10.31 (s, 1H), 10.16 (d, J=3.4 Hz, 1H), 7.82 (d, J=2.0 Hz, 1H), 7.61 (dd, J=8.3, 2.2 Hz, 1H), 7.44 (s, 1H), 7.28 (s, 1H), 7.25 (d, J=8.5 Hz, 1H), 6.88 (s, 1H), 6.67-6.63 (m, 1H), 3.55 (dd, J=9.6, 6.2 Hz, 1H), 3.51-3.43 (m, 3H), 3.16 (dd, J=9.6, 4.4 Hz, 1H), 2.66 (q, J=7.1 Hz, 2H), 2.50 (s, 3H), 2.20 (s, 3H), 2.17-2.11 (m, 1H), 1.91-1.83 (m, 1H), 1.23 (s, 1H), 1.05 (t, J=7.1 Hz, 3H).

HRMS (ESI+) calculated for C26H28F3N5O3 [M+H]+: 516.2217, found 516.6176

### 5-methyl-N-(2-methyl-5-(3-((1-methylpyrrolidin-3-yl)amino)-5-(trifluoromethyl)benzamido)phenyl)isoxazole-3-carboxamide (5o)

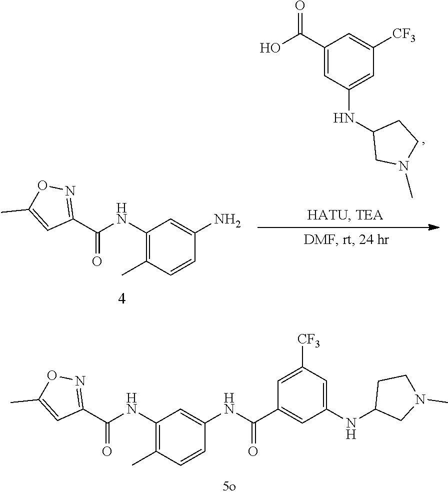

The reaction mixture was synthesized in the same manner as in the above 5g, and separated and purified through column chromatography to obtain 32.6 mg (0.065 mmol) of Compound 5o (Yield: 52.1%).

1H NMR (400 MHz, DMSO-d6) δ 10.31 (s, 1H), 10.19 (d, J=8.0 Hz, 1H), 7.82 (s, 1H), 7.60 (d, J=8.2 Hz, 1H), 7.40 (s, 1H), 7.32 (s, 1H), 7.25 (d, J=8.5 Hz, 1H), 7.00 (s, 1H), 6.66 (s, 1H), 6.61 (s, 1H), 4.06 (s, 1H), 2.88 (s, 1H), 2.71 (s, 1H), 2.52 (s, 1H), 2.51 (s, 3H), 2.34 (s, 3H), 2.28 (dd, J=13.2, 5.5 Hz, 2H), 2.19 (s, 3H), 1.65 (d, J=6.0 Hz, 1H).

### N-(5-(3-(3-(dimethylamino)pyrrolidin-1-yl)-5-(trifluoromethyl)benzamido)-2-methylphenyl)-5-methylisoxazole-3-carboxamide (5p)

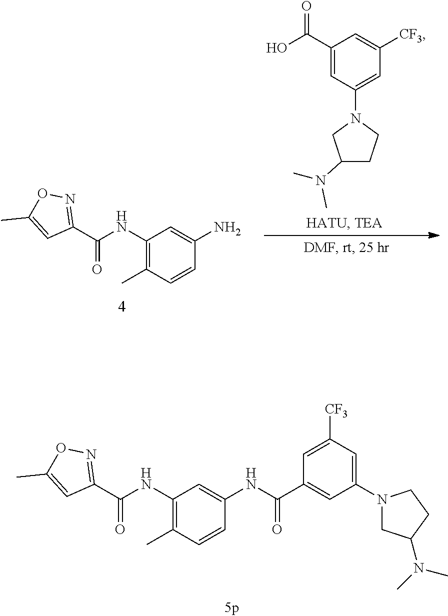

The obtained compound was synthesized in the same manner as in the above 5g, and was separated and purified through column chromatography to obtain Compound 5p (15.1 mg, 0.0293 mmol, 18.3%).

1H NMR (400 MHz, DMSO-d6) δ 10.30 (s, 1H), 10.17 (s, 1H), 7.82 (s, 1H), 7.62 (dd, J=8.3, 2.2 Hz, 1H), 7.44 (s, 1H), 7.30 (s, 1H), 7.26 (d, J=8.4 Hz, 1H), 6.92 (s, 1H), 6.66 (s, 1H), 3.55 (dt, J=29.5, 8.9 Hz, 2H), 3.35 (s, 1H), 3.18-3.12 (m, 1H), 2.84 (s, 1H), 2.49 (s, 3H), 2.24-2.18 (m, 10H), 1.85 (s, 1H).

### 5-methyl-N-(2-methyl-5-(quinoline-2-carboxamido)phenyl)isoxazole-3-carboxamide (5q)

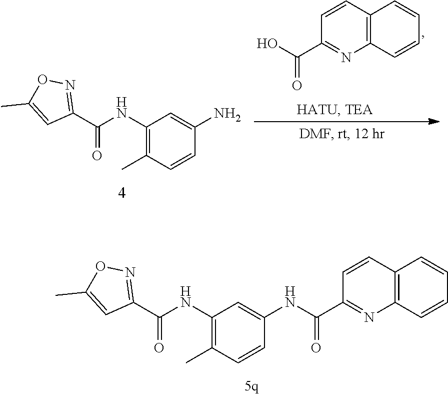 [SMILES: Cc1cc(C(=O)Nc2cc(N)ccc2C)no1 ; O=C(O)c1ccc2ccccc2n1 ; Cc1cc(C(=O)Nc2cc(NC(=O)c3ccc4ccccc4n3)ccc2C)no1]

The reaction mixture was synthesized in the same manner as in the above 5g, and separated and purified through column chromatography to obtain Compound 5q (7.1 mg, 0.018 mmol, 21.4%).

1H NMR (400 MHz, DMSO-d6) δ 10.76 (s, 1H), 10.22 (s, 1H), 8.64 (d, J=8.3 Hz, 1H), 8.25 (t, J=9.3 Hz, 2H), 8.13 (d, J=7.6 Hz, 1H), 8.05 (d, J=1.9 Hz, 1H), 7.92 (ddd, J=8.4, 6.9, 1.4 Hz, 1H), 7.82-7.73 (m, 2H), 7.30 (d, J=8.4 Hz, 1H), 6.68 (s, 1H), 2.52 (s, 3H), 2.22 (s, 3H).

HRMS (ESI+) calculated for C22H19N4O3 [M+H]+: 387.1452, found 387.5132

### N-(5-(1H-benzo[d][1,2,3]triazole-6-carboxamido)-2-methylphenyl)-5-methylisoxazole-3-carboxamide (5r)

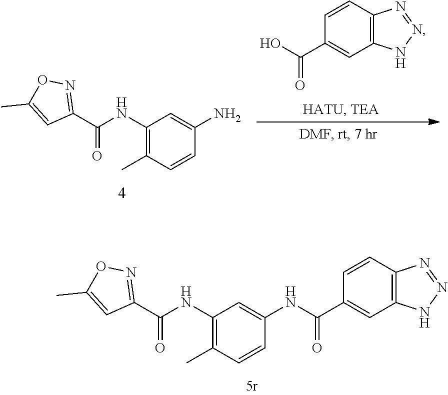 [SMILES: Cc1cc(C(=O)Nc2cc(N)ccc2C)no1 ; O=C(O)c1ccc2nn[nH]c2c1 ; Cc1cc(C(=O)Nc2cc(NC(=O)c3ccc4nn[nH]c4c3)ccc2C)no1]

The reaction mixture was synthesized in the same manner as in the above 5g, and separated and purified through column chromatography to obtain 2 mg (0.0053 mmol) of Compound 5r (yield: 10.2%).

1H NMR (400 MHz, DMSO-d6) δ 16.00 (s, 1H), 10.45 (s, 1H), 10.19 (s, 1H), 8.76 (s, 1H), 8.09 (s, 1H), 7.91 (s, 2H), 7.66 (dd, J=8.2, 2.1 Hz, 1H), 7.27 (d, J=8.6 Hz, 1H), 6.68 (s, 1H), 2.52 (s, 3H), 2.21 (s, 3H).

HRMS (ESI+) calculated for C19H16N6O3 [M+H]+: 377.1357, found 377.5012

### 5-methyl-N-(2-methyl-5-(1-phenyl-5-(trifluoromethyl)-1H-pyrazole-4-carboxamido)phenyl)isoxazole-3-carboxamide (5s)

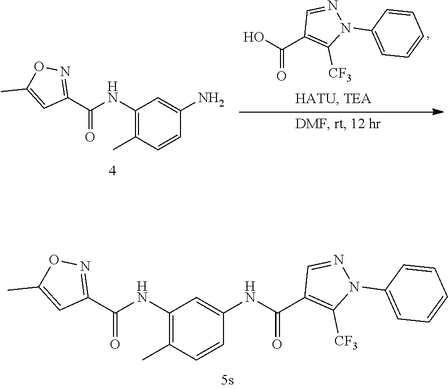 [SMILES: Cc1cc(C(=O)Nc2cc(N)ccc2C)no1 ; O=C(O)c1cnn(-c2ccccc2)c1C(F)(F)F ; Cc1cc(C(=O)Nc2cc(NC(=O)c3cnn(-c4ccccc4)c3C(F)(F)F)ccc2C)no1]

This compound was synthesized in the same manner as in the above 5g, and was separated and purified through column chromatography to obtain compound 5s (10.4 mg, 0.022 mmol, 31.7%).

1H NMR (400 MHz, DMSO-d6) δ 10.53 (s, 1H), 10.18 (s, 1H), 8.31 (s, 1H), 7.80 (s, 1H), 7.62-7.59 (m, 3H), 7.56-7.50 (m, 3H), 7.25 (d, J=8.6 Hz, 1H), 6.66 (s, 1H), 2.51 (s, 3H), 2.19 (s, 3H).

HRMS (ESI+) calculated for C23H18F3N5O3 [M+H]+: 470.1435, found 470.5232

### N-(5-(1H-indazole-6-carboxamido)-2-methylphenyl)-5-methylisoxazole-3-carboxamide (5t)

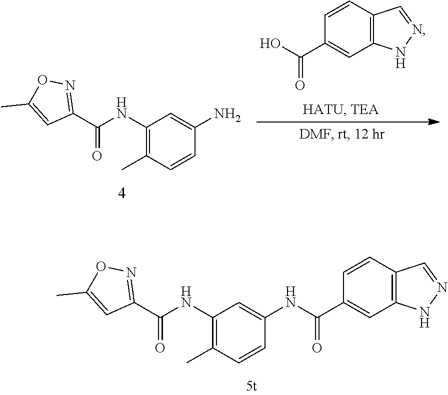 [SMILES: Cc1cc(C(=O)Nc2cc(N)ccc2C)no1 ; O=C(O)c1ccc2cn[nH]c2c1 ; Cc1cc(C(=O)Nc2cc(NC(=O)c3ccc4cn[nH]c4c3)ccc2C)no1]

The reaction mixture was synthesized in the same manner as in the above 5g, and separated and purified through column chromatography to obtain Compound 5t (4 mg, 0.0107 mmol, 15.2%).

1H NMR (400 MHz, DMSO-d6) δ 13.42 (s, 1H), 10.38 (s, 1H), 10.18 (s, 1H), 8.17 (d, J=6.2 Hz, 2H), 7.91-7.86 (m, 2H), 7.70-7.61 (m, 3H), 7.25 (d, J=8.5 Hz, 1H), 6.67 (s, 1H), 2.51 (s, 3H), 2.20 (s, 3H).

HRMS (ESI+) calculated for C20H17N5O3 [M+H]+: 376.1404, found 376.4928

### N-(5-(3-Chlorobenzamido)-2-methylphenyl)-5-methylisoxazole-3-carboxamide (5u)

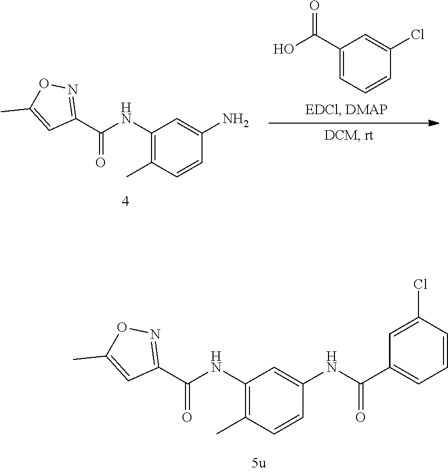 [SMILES: Cc1cc(C(=O)Nc2cc(N)ccc2C)no1 ; O=C(O)c1cccc(Cl)c1 ; Cc1cc(C(=O)Nc2cc(NC(=O)c3cccc(Cl)c3)ccc2C)no1]

After introducing compound 4 (19 mg, 0.082 mmol) and Dichloromethane (0.82 mL), 3-chlorobenzoic acid (15.3 mg, 0.098 mmol), EDCI (18.8 mg, 0.098 mmol), and DMAP (12 mg, 0.098 mmol) were added thereto, the mixture was stirred at room temperature at overnight. After confirming the completion of the reaction, Dichloromethane was extracted and washed with water. The resultant was dried with Anhydrous Na2SO4, distilled under reduced pressure, and concentrated. Separation and purification by column chromatography yielded Compound 5u (12 mg, 0.032 mmol, 39%).

1H NMR (400 MHz, DMSO-d6) δ 10.36 (s, 1H), 10.17 (s, 1H), 8.01 (t, J=1.8 Hz, 1H), 7.93-7.90 (m, 1H), 7.85 (d, J=2.9 Hz, 1H), 7.66 (ddd, J=8.0, 2.1, 1.1 Hz, 1H), 7.60 (dd, J=8.4, 2.5 Hz, 1H), 7.56 (d, J=7.9 Hz, 1H), 7.25 (d, J=8.6 Hz, 1H), 6.66 (s, 1H), 2.51 (s, 3H), 2.20 (s, 3H).

HRMS(ESI+) calculated for C19H17CIN3O3 [M+H]+: 370.0953, found 370.4063.

### 5-Methyl-N-(2-methyl-5-(3-((1-methylpiperidin-4-yl)oxy)-5-(trifluoromethyl)benzamido) phenyl)isoxazole-3-carboxamide (5v)

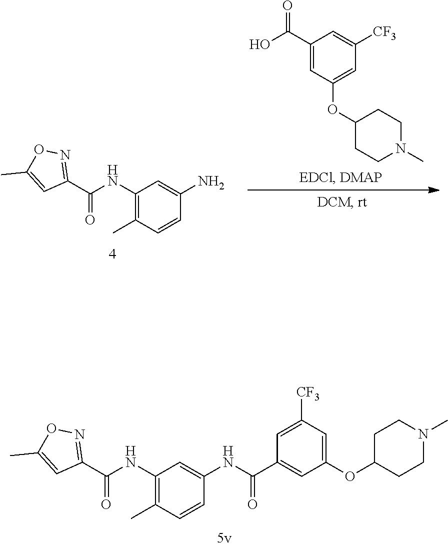

The reaction mixture was synthesized in the same manner as above, and separated and purified through column chromatography to obtain 9.1 mg (0.017 mmol, 20%) of Compound 5v.

1H NMR (400 MHz, MeOD) δ 7.97-7.94 (m, 1H), 7.87-7.84 (m, 1H), 7.80-7.78 (m, 1H), 7.64-7.58 (m, 1H), 7.45-7.42 (m, 1H), 7.35-7.31 (m, 1H), 6.61-6.57 (m, 1H), 4.74-4.65 (m, 1H), 2.86-2.78 (m, 2H), 2.60-2.49 (m, 5H), 2.43-2.39 (m, 3H), 2.34-2.31 (m, 3H), 2.14-2.06 (m, 2H), 1.97-1.89 (m, 2H).

HRMS(ESI+) calculated for C26H28F3N4O4 [M+H]+: 517.2057, found 517.5538.

### 2. Synthesis of 6-fluoro-2-methyl-3-nitropyridine derivatives 6-methyl-5-nitro-N-(tetrahydro-2H-pyran-4-yl)pyridin-2-amine (15b)

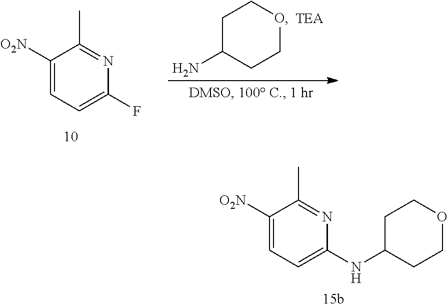 [SMILES: Cc1nc(F)ccc1[N+](=O)[O-] ; NC1CCOCC1 ; Cc1nc(NC2CCOCC2)ccc1[N+](=O)[O-]]

Compound 148 (l 1.28 mmol), DMSO (6.4 mL), tetrahydro-2H-pyran-4-amine, TEA (356 l, 2.56 mmol) were added thereto, and the mixture was stirred at 100° C. for 2 hours.

After completion of the reaction, the reaction mixture was diluted in EA and washed with DW, Brine. In order to remove DMSO remaining in the EA layer, the resultant product was washed with Brine via No. 6 to No. 7, and moisture of the EA layer was removed using Na2SO4, and the resultant product was distilled under reduced pressure, and separated and purified by column chromatography to obtain target Compound 15b (79.1 mg, 104%).

1H NMR (400 MHz, DMSO-d6) δ 8.09 (d, J=9.0 Hz, 1H), 7.90 (d, J=7.6 Hz, 1H), 6.45 (d, J=9.3 Hz, 1H), 4.23-4.01 (m, 1H), 3.91-3.83 (m, 2H), 3.41 (td, J=11.6, 2.2 Hz, 2H), 2.65 (s, 3H), 1.86 (d, J=11.5 Hz, 2H), 1.48 (dt, J=19.7, 7.6 Hz, 2H).

HRMS (ESI+) calculated for C11H15N3O3 [M+H]+: 238.1186, found 238.6544

### 6-methyl-N2-(tetrahydro-2H-pyran-4-yl)pyridine-2,5-diamine (16b)

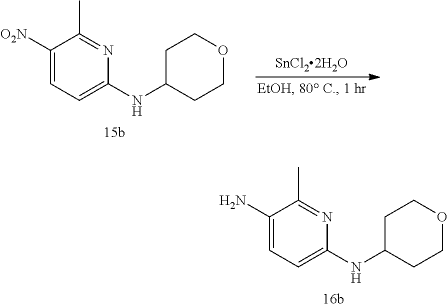 [SMILES: Cc1nc(NC2CCOCC2)ccc1[N+](=O)[O-] ; Cc1nc(NC2CCOCC2)ccc1N]

Compound 15b (100 mg, 0.448 mmol), EtOH (0.896 mL), and SnCl2 (425 mg, 2.24 mmol) were added thereto, and the mixture was stirred at 80° C. for 1 hour. After completion of the reaction, the reaction mixture was diluted in EA and washed with NaHCO₃ and Brine. The moisture of the EA layer was removed using Na2SO4, distilled under reduced pressure, and separated and purified using column chromatography to obtain the desired Compound 16b (21.4 mg, 81.7%).

1H NMR (400 MHz, DMSO-d6) δ 6.78 (d, J=8.4 Hz, 1H), 6.15 (d, J=8.4 Hz, 1H), 5.30 (d, J=8.1 Hz, 1H), 4.12-3.96 (m, 2H), 3.83 (dt, J=6.9, 3.6 Hz, 2H), 3.69-3.61 (m, 1H), 3.38 (dd, J=11.5, 2.2 Hz, 2H), 2.11 (s, 3H), 1.83 (d, J=10.3 Hz, 2H), 1.38-1.29 (m, 2H).

HRMS (ESI+) calculated for C11H17N3O [M+2H]+: 208.1444, found 209.3333

### 5-methyl-N-(2-methyl-6-((tetrahydro-2H-pyran-4-yl)amino)pyridin-3-yl)isoxazole-3-carboxamide (17b)

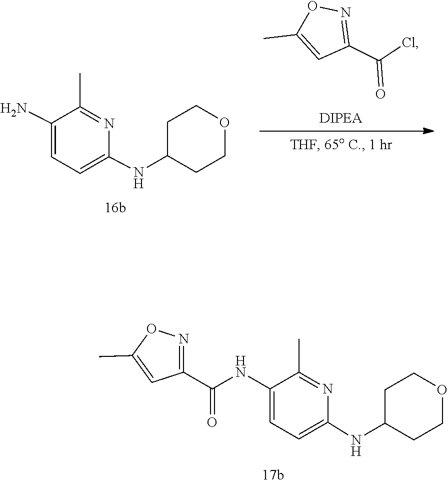

After Compound 16b was dissolved in THE (1 mL), 5-methylisoxazole-3-carbonyl chloride was dissolved in THE (1.18 mL), dropwise was performed, DIPEA (74.2f) was added thereto, and the resulting mixture was stirred at 65° C. for 1 hour. After completion of the reaction, the reaction mixture was diluted with EA and washed with NaHCO₃, Brine. The moisture of the EA layer was removed by Na2SO4 and distilled under reduced pressure. The result was purified with column chromatography to obtain target Compound 17b (16.2 mg, 0.0512 mmol, 50.7%).

1H NMR (400 MHz, DMSO-d6) δ 9.94 (s, 1H), 7.22 (d, J=8.6 Hz, 1H), 6.60 (d, J=0.9 Hz, 1H), 6.41 (d, J=7.7 Hz, 1H), 6.32 (d, J=8.6 Hz, 1H), 3.86 (dd, J=8.1, 3.2 Hz, 3H), 3.40 (td, J=11.5, 2.1 Hz, 2H), 2.49 (d, J=0.8 Hz, 3H), 2.17 (s, 3H), 1.91-1.83 (m, 2H), 1.41 (ddd, J=15.5, 12.0, 4.3 Hz, 2H).

HRMS (ESI+) calculated for C16H20N4O3 [M+H]+: 317.1608, found 317.6430

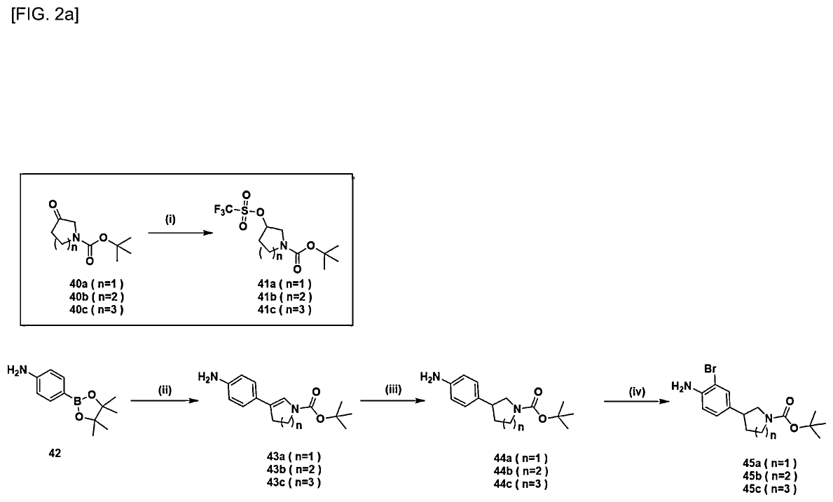

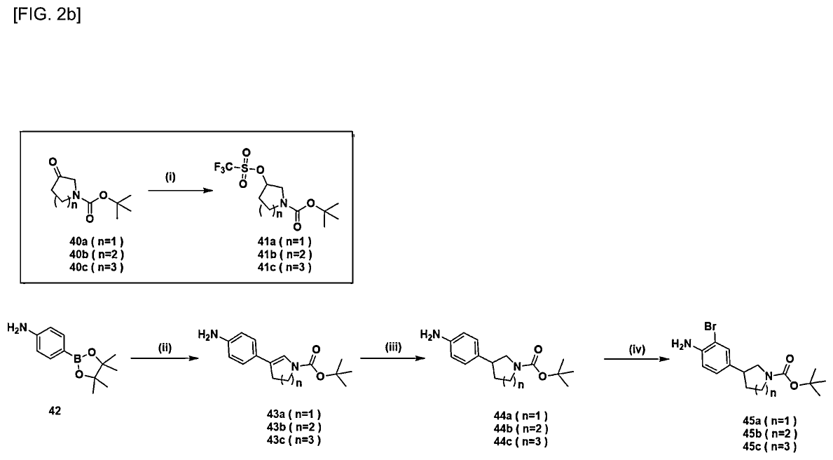

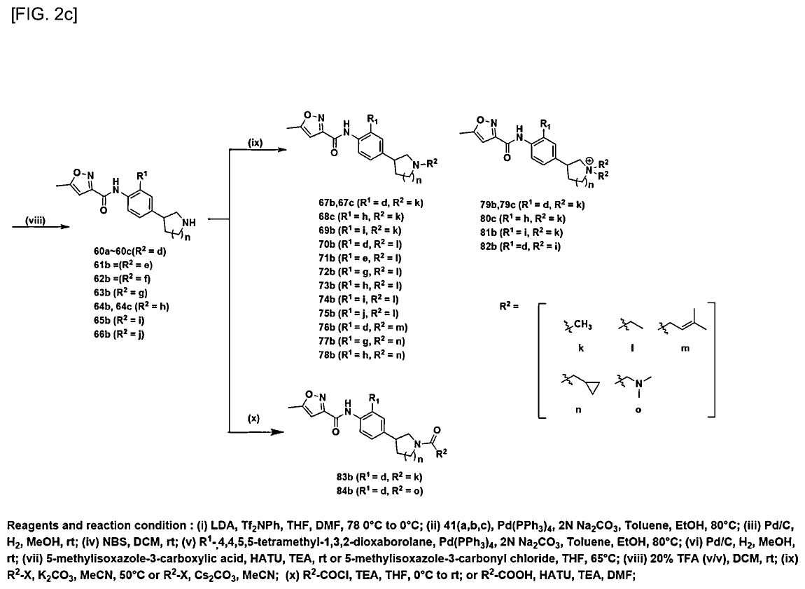

3. Synthesis of 5-methylisoxazole-3-carboxamide derivatives (Scheme 2, FIGS. 2a-2c)

### 5-methylisoxazole-3-carboxamide derivatives

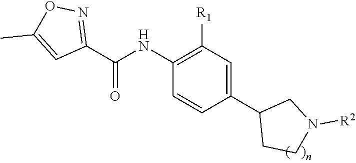

IC50<100 nM:+++, 1 μM>IC50>100 nM: ++, IC50>1 μM: +

TABLE 1

| | n = | | | |
|---|---|---|---|---|
| NO. | 1, 2, 3 | R¹ | R² | IC₅₀ |
| 60a | n = 1 | 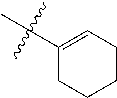 [SMILES: *C1=CCCCC1] | H | ++ |
| 60b | n = 2 | 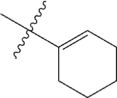 [SMILES: *C1=CCCCC1] | H | +++ |
| 60c | n = 3 | 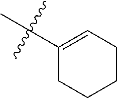 [SMILES: *C1=CCCCC1] | H | +++ |
| 61b | n = 2 | 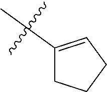 [SMILES: *C1=CCCC1] | H | + |
| 62b | n = 2 | 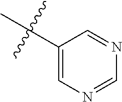 [SMILES: *c1cncnc1] | H | + |
| 63b | n = 2 | 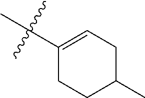 [SMILES: *C1=CCC(C)CC1] | H | +++ |
| 64b | n = 2 | 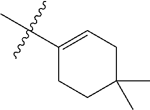 [SMILES: *C1=CCC(C)(C)CC1] | H | +++ |
| 64c | n = 3 | 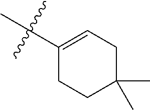 [SMILES: *C1=CCC(C)(C)CC1] | H | +++ |
| 65b | n = 2 | 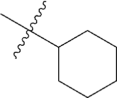 [SMILES: *C1CCCCC1] | H | + |
| 66b | n = 2 | 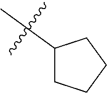 [SMILES: *C1CCCC1] | H | + |
| 67b | n = 2 | 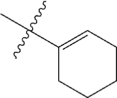 [SMILES: *C1=CCCCC1] | 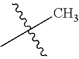 | +++ |
| 67c | n = 3 | 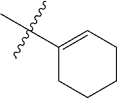 [SMILES: *C1=CCCCC1] | 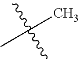 | ++ |
| 68c | n = 3 | 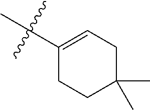 [SMILES: *C1=CCC(C)(C)CC1] | 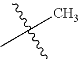 | +++ |
| 69b | n = 2 | 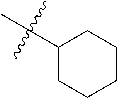 [SMILES: *C1CCCCC1] | 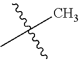 | + |
| 71b | n = 2 | 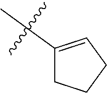 [SMILES: *C1=CCCC1] | 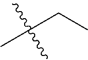 [SMILES: *CC] | + |
| 72b | n = 2 | 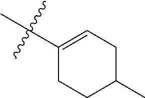 [SMILES: *C1=CCC(C)CC1] | 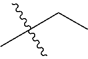 [SMILES: *CC] | +++ |
| 73b | n = 2 | 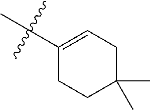 [SMILES: *C1=CCC(C)(C)CC1] | 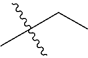 [SMILES: *CC] | +++ |
| 74b | n = 2 | 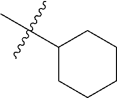 [SMILES: *C1CCCCC1] | 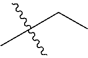 [SMILES: *CC] | + |
| 75b | n = 2 | 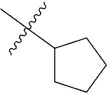 [SMILES: *C1CCCC1] | 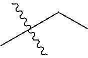 [SMILES: *CC] | + |
| 76b | n = 2 | 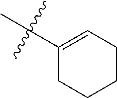 [SMILES: *C1=CCCCC1] | 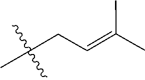 [SMILES: *CC=C(C)C] | ++ |
| 77b | n = 2 | 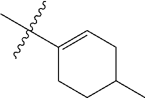 [SMILES: *C1=CCC(C)CC1] | 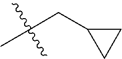 [SMILES: *CC1CC1] | +++ |
| 78b | n = 2 | 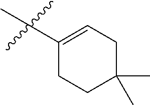 [SMILES: *C1=CCC(C)(C)CC1] | 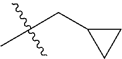 [SMILES: *CC1CC1] | +++ |

### Methods for Synthesis of 5-methylisoxazole-3-carboxamide Derivatives

### Tert-butyl 3-(((trifluoromethyl)sulfonyl)oxy)-2,5-dihydro-1H-pyrrole-1-carboxylate. (41a)

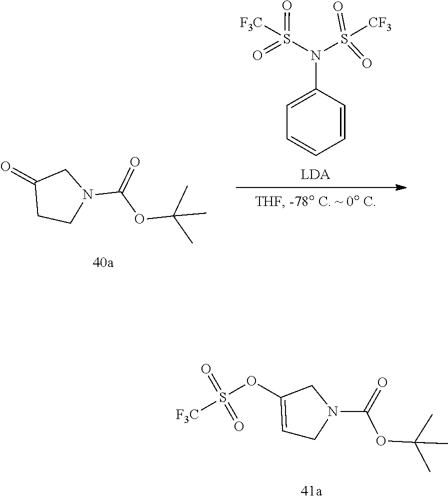 [SMILES: O=S(=O)(N(c1ccccc1)S(=O)(=O)C(F)(F)F)C(F)(F)F ; CC(C)(C)OC(=O)N1CCC(=O)C1 ; CC(C)(C)OC(=O)N1CC=C(OS(=O)(=O)C(F)(F)F)C1]

At −78° C., 2M LDA (1.62 mL) was diluted in THE (4 mL), and 1-Boc-3-pyrrolidone (500 mg, 2.7 mmol) was dissolved in THE (4 mL), dropped, and stirred for 10 minutes. N-Phenylbis (trifluoromethanesulfonimide) (1.143 g, 3.24 mmol) was dissolved in THE (5.5 mL), and the resulting solution was slowly added thereto, followed by stirring at 12h-24h. After being transferred to room temperature, the reaction mixture was extracted with Ethyl acetate, washed with water, NH4Cl, brine, dried over Anhydrous Na2SO4, and concentrated. Separation and purification by column chromatography yielded Compound 41a (130 mg, 15%).

1H NMR (400 MHz, CDCl3) δ 5.71 (s, 1H), 4.24 (d, J=4.9 Hz, 2H), 4.19 (d, J=5.2 Hz, 2H), 1.48 (s, 9H).

HRMS(ESI+) calculated for C10H14F3NNaO5S [M+Na]+: 340.0437, found 340.3694.

### Tert-butyl 4-(((trifluoromethyl)sulfonyl)oxy)-3,6-dihydropyridine-1(2H)-carboxylate. (41b)

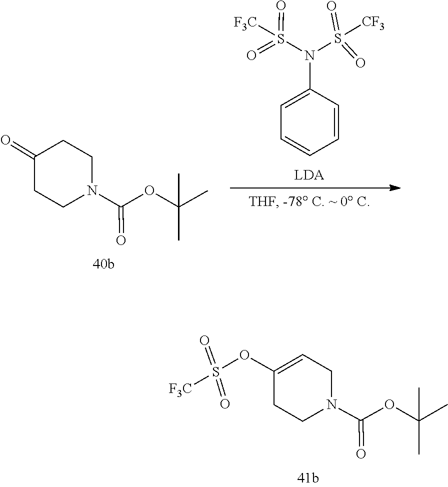 [SMILES: CC(C)(C)OC(=O)N1CCC(=O)CC1 ; O=S(=O)(N(c1ccccc1)S(=O)(=O)C(F)(F)F)C(F)(F)F ; CC(C)(C)OC(=O)N1CC=C(OS(=O)(=O)C(F)(F)F)CC1]

The reaction mixture was synthesized in the same manner as above, and separated and purified through column chromatography to obtain 784.6 mg (94%) of Compound 41 b.

1H NMR (400 MHz, DMSO-d6) δ 6.02 (s, 1H), 3.98 (d, J=3.1 Hz, 2H), 3.54 (t, J=5.8 Hz, 2H), 2.44-2.39 (m, 2H), 1.41 (s, 9H).

HRMS(ESI+) calculated for C11H16F3NO5S [M+Na]+: 354.0593, found 354.3435.

### Tert-butyl 5-(((trifluoromethyl)sulfonyl)oxy)-2,3,4,7-tetrahydro-1H-azepine-1-carboxylate. (41c)

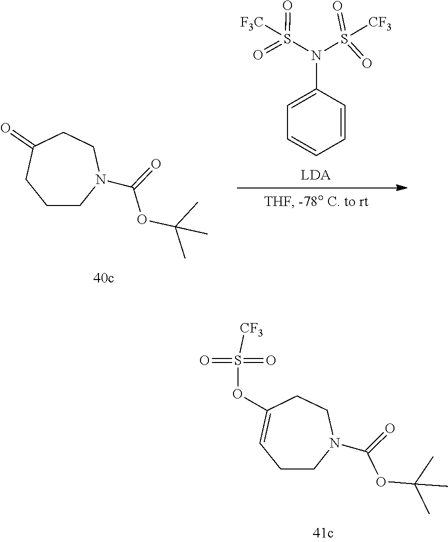 [SMILES: CC(C)(C)OC(=O)N1CCCC(=O)CC1 ; O=S(=O)(N(c1ccccc1)S(=O)(=O)C(F)(F)F)C(F)(F)F ; CC(C)(C)OC(=O)N1CCC=C(OS(=O)(=O)C(F)(F)F)CC1]

The reaction mixture was synthesized in the same manner as above, and separated and purified through column chromatography to obtain 1.949 mg (104%) of Compound 41c.

1H NMR (400 MHz, DMSO-d6) δ 6.05 (t, J=5.1 Hz, 1H), 3.89 (s, 2H), 3.49 (dd, J=5.7, 3.2 Hz, 2H), 2.66 (s, 1H), 2.53 (dd, J=7.1, 4.1 Hz, 1H), 2.32 (dt, J=5.9, 3.0 Hz, 1H), 1.85 (d, J=5.4 Hz, 2H), 1.38 (d, J=3.7 Hz, 9H).

HRMS(ESI+) calculated for C12H18F3NNaO5S [M+Na]+: 368.0750, found 368.3174.

### Tert-butyl 3-(4-aminophenyl)-2,5-dihydro-1H-pyrrole-1-carboxylate. (43a)

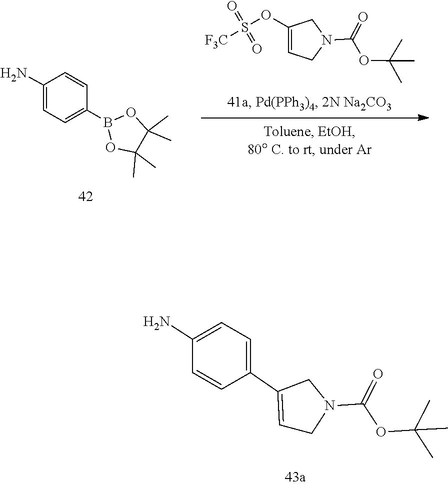 [SMILES: CC1(C)OB(c2ccc(N)cc2)OC1(C)C ; CC(C)(C)OC(=O)N1CC=C(OS(=O)(=O)C(F)(F)F)C1 ; CC(C)(C)OC(=O)N1CC=C(c2ccc(N)cc2)C1]

Compound 41a (130 mg, 0.40 mmol) obtained above and Pd(PPh3)4 (38 mg, 0.04 mmol) were added and dissolved using Toluene (2.94 mL) and Ethanol (1.46 mL), 2N Na2CO3 (1.45 mL, 2.9 mmol) was added thereto, and 4-(4, 4, 5, 5-tetramethyl-1, 3, 2-dioxaborolan-2-yl)aniline (72 mg, 0.33 mmol) was added thereto at 80° C., followed by stirring for 2 hours. After confirming the completion of the reaction, the reaction mixture was transferred to room temperature and cooled, and then extracted with Ethyl acetate, and washed with NaHCO₃, water, and brine. It is directed to drying with Anhydrous Na2SO4, concentrating, separation and purification using column chromatography (EA:HEX=1:5) to afford compound 43a (66 mg, 76%)

1H NMR (400 MHz, DMSO-d6) δ 7.12 (dd, J=8.5, 3.4 Hz, 2H), 6.56-6.51 (m, 2H), 5.96 (d, J=14.3 Hz, 1H), 5.27 (s, 2H), 4.33-4.26 (m, 2H), 4.13 (d, J=2.4 Hz, 2H), 1.44 (d, J=5.6 Hz, 9H).

HRMS(ESI+) calculated for C15H20N2NaO2 [M+Na]+: 283.1417, found 283.3903.

### Tert-butyl 4-(4-aminophenyl)-3,6-dihydropyridine-1(2H)-carboxylate. (43b)

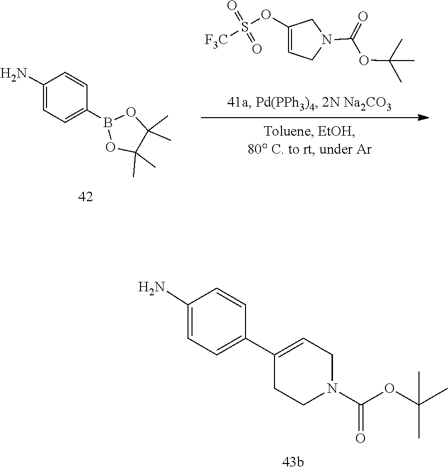 [SMILES: CC1(C)OB(c2ccc(N)cc2)OC1(C)C ; CC(C)(C)OC(=O)N1CC=C(OS(=O)(=O)C(F)(F)F)C1 ; CC(C)(C)OC(=O)N1CC=C(c2ccc(N)cc2)CC1]

The reaction mixture was synthesized in the same manner as above, and separated and purified through column chromatography to obtain 380 mg (70%) of Compound 43b.

1H NMR (400 MHz, DMSO-d6) δ 7.15-7.08 (m, 2H), 6.55-6.47 (m, 2H), 5.88 (s, 1H), 5.11 (s, 2H), 3.93 (s, 2H), 3.49 (t, J=5.7 Hz, 2H), 2.35 (t, J=7.4 Hz, 2H), 1.42 (s, 9H).

HRMS (ESI+) calculated for C16H22N2NaO2 [M+Na]+:297.1573, found 297.3293.

### Tert-butyl 5-(4-aminophenyl)-2,3,4,7-tetrahydro-1H-azepine-1-carboxylate. (43c)

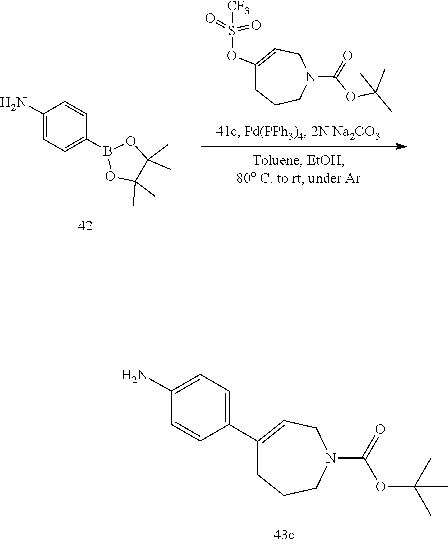 [SMILES: CC1(C)OB(c2ccc(N)cc2)OC1(C)C ; CC(C)(C)OC(=O)N1CC=C(OS(=O)(=O)C(F)(F)F)CCC1 ; CC(C)(C)OC(=O)N1CC=C(c2ccc(N)cc2)CCC1]

The reaction mixture was synthesized in the same manner as above, and separated and purified through column chromatography to obtain 699 mg (43%) of Compound 43c.

1H NMR (400 MHz, DMSO-d6) δ 7.02 (dd, J=8.6, 2.6 Hz, 2H), 6.53-6.48 (m, 2H), 5.83 (dd, J=12.6, 6.3 Hz, 1H), 5.35 (s, 2H), 3.89 (s, 2H), 3.50 (t, J=5.9 Hz, 2H), 2.54 (dd, J=11.0, 5.4 Hz, 2H), 1.74 (s, 2H), 1.39 (d, J=8.1 Hz, 9H).

HRMS(ESI+) calculated for C17H24N2NaO2 [M+Na]+: 311.1730, found 311.4121.

### Tert-butyl 3-(4-aminophenyl)pyrrolidine-1-carboxylate. (44a)

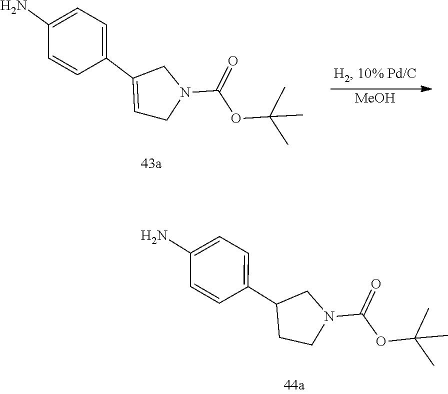 [SMILES: "MeOH" ; CC(C)(C)OC(=O)N1CC=C(c2ccc(N)cc2)C1 ; CC(C)(C)OC(=O)N1CCC(c2ccc(N)cc2)C1]

Compound 43a (66 mg, 0.25 mmol) obtained above was dissolved in Methanol (8.3 mL), Pd/C (6.6 mg) was added thereto, and the mixture was substituted with hydrogen gas and stirred for 1 hour. After the completion of the reaction was confirmed, Pd/C was removed using cellite and concentrated to give Compound 44a (70 mg, 106%).

1H NMR (400 MHz, DMSO-d6) δ 6.91 (d, J=8.3 Hz, 2H), 6.54-6.47 (m, 2H), 4.89 (d, J=8.8 Hz, 2H), 3.59 (dd, J=10.0, 7.4 Hz, 1H), 3.42 (t, J=9.2 Hz, 1H), 3.28-3.20 (m, 1H), 3.15 (dd, J=14.2, 4.2 Hz, 1H), 3.02 (t, J=9.1 Hz, 1H), 2.06 (d, J=6.4 Hz, 1H), 1.84 (dt, J=18.8, 9.3 Hz, 1H), 1.40 (d, J=4.6 Hz, 9H).

HRMS(ESI+) calculated for C15H22N2NaO2 [M+Na]+: 285.1573, found 285.4433.

### Tert-butyl 4-(4-aminophenyl)piperidine-1-carboxylate. (44b)

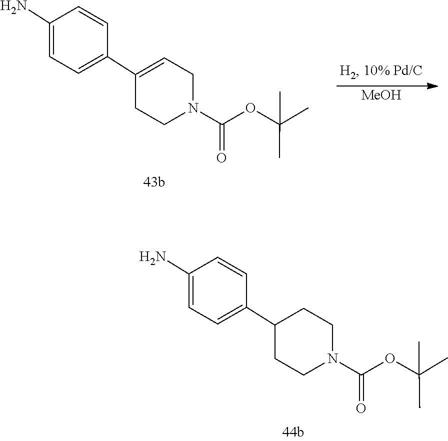 [SMILES: "MeOH" ; CC(C)(C)OC(=O)N1CC=C(c2ccc(N)cc2)CC1 ; CC(C)(C)OC(=O)N1CCC(c2ccc(N)cc2)CC1]

Compound 44b (634 mg, 99%) was synthesized in the same manner as in the above without further purification.

1H NMR (400 MHz, DMSO-d6) δ 6.86 (d, J=8.4 Hz, 2H), 6.48 (t, J=5.5 Hz, 2H), 4.88 (s, 2H), 4.03 (dd, J=14.2, 7.1 Hz, 2H), 2.75 (s, 2H), 2.47-2.41 (m, 1H), 1.66 (d, J=12.7 Hz, 2H), 1.41 (s, 9H), 1.39-1.34 (m, 2H).

HRMS (ESI+) calculated for C16H24N2NaO2 [M+Na]+:299.1730, found 299.9102.

### Tert-butyl 4-(4-aminophenyl)azepane-1-carboxylate. (44c)

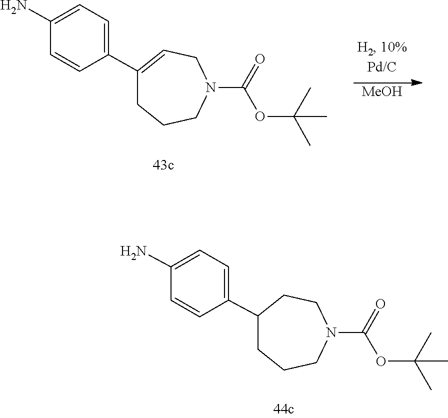 [SMILES: "MeOH" ; CC(C)(C)OC(=O)N1CC=C(c2ccc(N)cc2)CCC1 ; CC(C)(C)OC(=O)N1CCCC(c2ccc(N)cc2)CC1]

a compound 44c (416 mg, 59%) obtained by the same method as above without further purification

1H NMR (400 MHz, DMSO-d6) δ 6.81 (d, J=8.4 Hz, 2H), 6.46 (d, J=8.0 Hz, 2H), 4.78 (d, J=9.2 Hz, 2H), 3.53 (dd, J=18.7, 14.3 Hz, 2H), 3.45-3.36 (m, 2H), 2.41 (s, 1H), 1.80 (s, 2H), 1.73 (s, 1H), 1.60 (s, 2H), 1.49 (d, J=11.0 Hz, 1H), 1.41 (d, J=1.5 Hz, 9H).

HRMS(ESI+) calculated for C17H26N2NaO2 [M+Na]+: 313.1886, found 313.4290.

### Tert-butyl 3-(4-amino-3-bromophenyl)pyrrolidine-1-carboxylate. (45a)

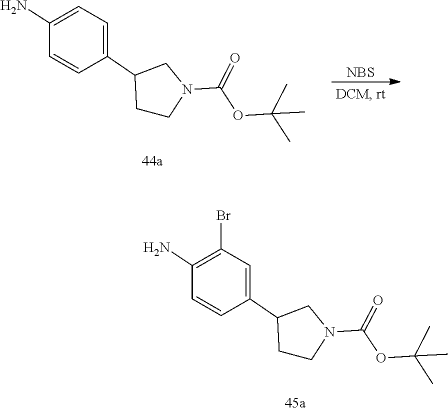

Compound 44a (70 mg. 0.26 mmol) was dissolved in DCM, and N-Bromosuccinimde (46 mg, 0.26 mmol) was slowly added to the reaction solution in 4 times at 30-minute intervals. After confirming the completion of the reaction, the reaction mixture was extracted with Ethyl acetate and washed with water and brine. It was dried over Anhydrous Na2SO4, concentrated, and separated and purified using column chromatography to obtain 55 mg (61%) of Compound 45a.

1H NMR (400 MHz, MeOD) δ 7.27 (d, J=1.9 Hz, 1H), 7.01 (dd, J=8.3, 2.0 Hz, 1H), 6.81-6.77 (m, 1H), 3.73-3.65 (m, 1H), 3.53 (t, J=9.4 Hz, 1H), 3.36 (dd, J=17.2, 9.4 Hz, 1H), 3.23 (dd, J=9.5, 6.5 Hz, 1H), 3.21-3.14 (m, 1H), 2.18 (dd, J=9.0, 6.2 Hz, 1H), 1.99-1.86 (m, 1H), 1.47 (s, 9H).

HRMS(ESI+) calculated for C15H21BrN2NaO2 [M+Na]+: 363.0697, found 363.4193.

### Tert-butyl 4-(4-amino-3-bromophenyl)piperidine-1-carboxylate. (45b)

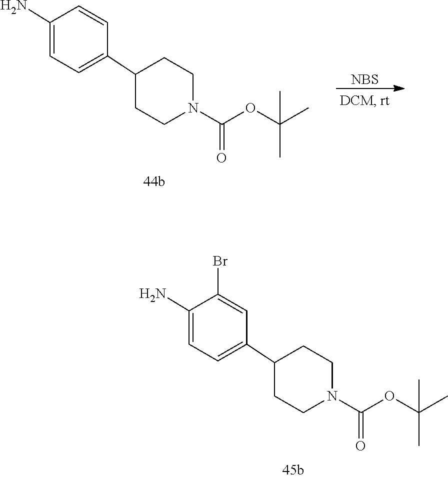

The reaction mixture was synthesized in the same manner as above, and separated and purified through column chromatography to obtain 656 mg (80%) of Compound 45b.

1H NMR (400 MHz, DMSO-d6) δ 7.18 (d, J=1.9 Hz, 1H), 6.94 (dd, J=8.3, 2.0 Hz, 1H), 6.72 (d, J=8.2 Hz, 1H), 5.08 (d, J=11.1 Hz, 2H), 4.03 (dd, J=14.2, 7.1 Hz, 2H), 2.71 (d, J=29.4 Hz, 2H), 2.55-2.51 (m, 1H), 1.68 (d, J=12.8 Hz, 2H), 1.40 (s, 9H), 1.39-1.32 (m, 2H).

HRMS (ESI+) calculated for C16H23BrN2NaO2 [M+Na]+: 377.0835, found 377.3571.

### Tert-butyl 4-(4-amino-3-bromophenyl)azepane-1-carboxylate. (45c)

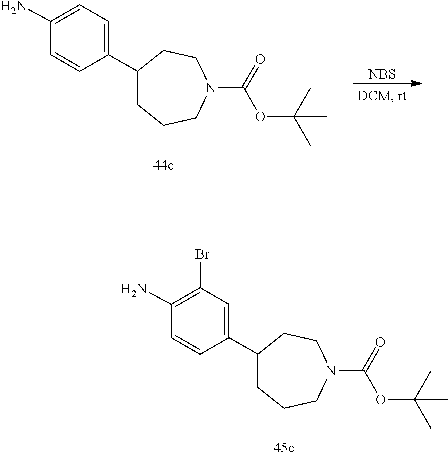

The reaction mixture was synthesized in the same manner as above, and separated and purified through column chromatography to obtain 423 mg (80%) of Compound 45c.

1H NMR (400 MHz, DMSO-d6) δ 7.13 (d, J=2.0 Hz, 1H), 6.88 (dd, J=8.3, 2.0 Hz, 1H), 6.71 (d, J=8.3 Hz, 1H), 5.06 (s, 2H), 3.53 (dd, J=18.5, 14.3 Hz, 1H), 3.45-3.34 (m, 2H), 3.20-3.12 (m, 1H), 2.44 (d, J=10.6 Hz, 1H), 1.81 (s, 2H), 1.73 (s, 1H), 1.66-1.55 (m, 2H), 1.50 (d, J=11.0 Hz, 1H), 1.41 (d, J=1.2 Hz, 9H).

HRMS(ESI+) calculated for C17H25BrN2NaO2 [M+Na]+: 391.0997, found 391.3307.

### Tert-butyl 3-(6-amino-2,3,4,5′-tetrahydro-[1,1′-biphenyl]-3-yl)pyrrolidine-1-carboxylate. (46a)

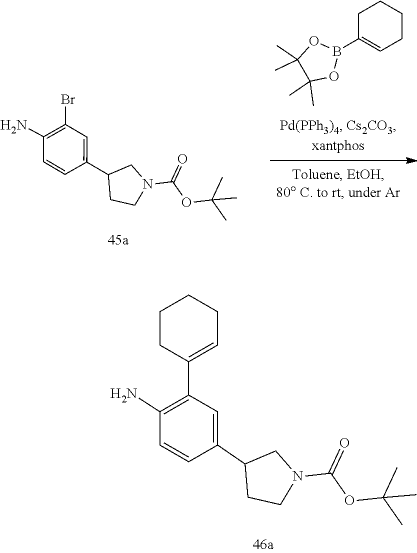 [SMILES: CC(C)(C)OC(=O)N1CCC(c2ccc(N)c(Br)c2)C1 ; CC1(C)OB(C2=CCCCC2)OC1(C)C ; CC(C)(C)OC(=O)N1CCC(c2ccc(N)c(C3=CCCCC3)c2)C1]

Compound 45a (55 mg, 0.16 mmol), Pd(PPh3)4 (18.4 mg, 0.016 mmol), xantphos (18.5 mg, 0.032 mmol), and Cs2CO3 (229 mg, 0.704 mmol) obtained above were dissolved in Toluene (1.33 mL) and Ethanol (0.66 mL), and 2-(1-Cyclohexyl)-4, 4, 5, 5-tetramethyl-1, 3, 2-dioxaborolane (45 μL, 0.208 mmol) was added dropwise and stirred at 80° C. After confirming the completion of the reaction, it was transferred to room temperature, extracted with Ethyl acetate, and washed with water and brine. It was dried over Anhydrous Na2SO4, concentrated, separated and purified using column chromatography to afford compound 46a (33 mg, 60%).

1H NMR (400 MHz, DMSO-d6) δ 6.83 (d, J=8.1 Hz, 1H), 6.72 (d, J=2.0 Hz, 1H), 6.57 (d, J=8.2 Hz, 1H), 5.60 (s, 1H), 4.54 (d, J=10.7 Hz, 2H), 3.58 (dd, J=9.9, 7.4 Hz, 1H), 3.41 (dd, J=10.8, 7.0 Hz, 1H), 3.25-3.19 (m, 1H), 3.14 (d, J=8.0 Hz, 1H), 3.04 (t, J=9.7 Hz, 1H), 2.13 (dd, J=8.5, 5.8 Hz, 5H), 1.82 (d, J=9.9 Hz, 1H), 1.70 (dd, J=7.6, 3.7 Hz, 2H), 1.65-1.60 (m, 2H), 1.40 (d, J=4.3 Hz, 9H).

HRMS(ESI+) calculated for C21H30N2NaO2 [M+Na]+: 365.2205, found 365.5803.

### Tert-butyl 4-(6-amino-2′,3′,4′,5′-tetrahydro-[1,1′-biphenyl]-3-yl)piperidine-1-carboxylate. (46b)

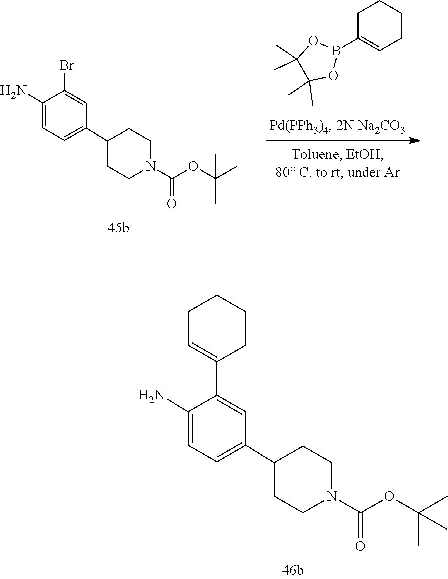 [SMILES: CC(C)(C)OC(=O)N1CCC(c2ccc(N)c(Br)c2)CC1 ; CC1(C)OB(C2=CCCCC2)OC1(C)C ; CC(C)(C)OC(=O)N1CCC(c2ccc(N)c(C3=CCCCC3)c2)CC1]

Compound 45b (200 mg, 0.56 mmol) obtained above, Pd(PPh3)4 (65 mg, 0.056 mmol), and 2N Na2CO3 (2.3 mL) were dissolved in Toluene (4.6 mL) and Ethanol (2.3 mL), and 2-(1-Cyclohexyl)-4, 4, 5, 5-tetramethyl-1, 3, 2-dioxaborolane (156 μL, 0.72 mmol) was added dropwise and stirred at 80° C. After confirming the completion of the reaction, the reaction mixture was transferred to room temperature, extracted with Ethyl acetate, and washed with water and brine. The resultant was dried over Anhydrous Na2SO4, concentrated, separated and purified using column chromatography to obtain 152 mg (75%) of Compound 46b.

1H NMR (400 MHz, DMSO-d6) δ 6.77 (dd, J=8.2, 2.1 Hz, 1H), 6.68 (d, J=2.1 Hz, 1H), 6.55 (d, J=8.1 Hz, 1H), 5.61-5.57 (m, 1H), 4.49 (s, 2H), 4.03 (dd, J=14.2, 7.1 Hz, 2H), 2.74 (s, 2H), 2.48-2.41 (m, 1H), 2.17-2.09 (m, 4H), 1.66 (ddd, J=13.3, 7.6, 4.8 Hz, 6H), 1.40 (s, 9H), 1.39-1.31 (m, 2H).

HRMS (ESI+) calculated for C22H32N2NaO2 [M+Na]+:379.2356, found 379.4460.

### Tert-butyl 4-(6-amino-2′,3′,4′,5′-tetrahydro-[1,1′-biphenyl]-3-yl)azepane-1-carboxylate. (46c)

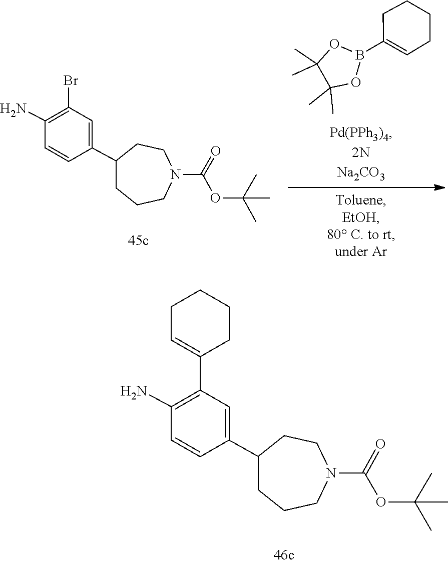 [SMILES: CC(C)(C)OC(=O)N1CCCC(c2ccc(N)c(Br)c2)CC1 ; CC1(C)OB(C2=CCCCC2)OC1(C)C ; CC(C)(C)OC(=O)N1CCCC(c2ccc(N)c(C3=CCCCC3)c2)CC1]

Compound 46c (62 mg, 79%) was synthesized in the same manner as in Example 46b, and separated and purified by column chromatography.

1H NMR (400 MHz, DMSO-d6) δ 6.76 (dd, J=8.2, 2.1 Hz, 1H), 6.67 (s, 1H), 6.57 (d, J=8.1 Hz, 1H), 5.61 (s, 1H), 4.70 (s, 2H), 3.55 (t, J=15.3 Hz, 1H), 3.42 (dd, J=11.4, 6.7 Hz, 2H), 3.19 (d, J=5.9 Hz, 1H), 2.48-2.41 (m, 1H), 2.15 (dd, J=7.9, 6.0 Hz, 4H), 1.80 (d, J=13.7 Hz, 3H), 1.76-1.69 (m, 3H), 1.65-1.62 (m, 2H), 1.57-1.49 (m, 2H), 1.43 (d, J=1.5 Hz, 9H).

HRMS(ESI+) calculated for C23H34N2NaO2 [M+Na]+: 393.2512, found 393.3115.

### Tert-butyl 4-(4-amino-3-(cyclopent-1-en-1-yl)phenyl)piperidine-1-carboxylate. (47b)

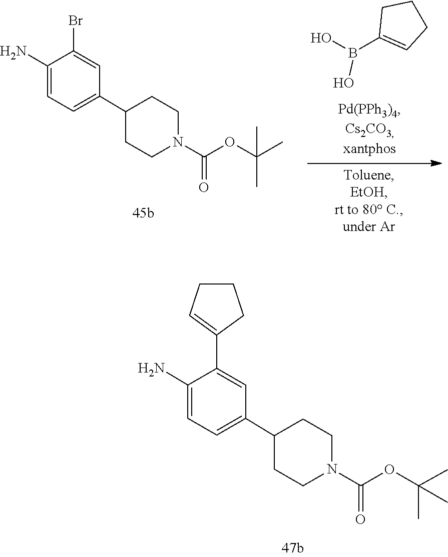 [SMILES: CC(C)(C)OC(=O)N1CCC(c2ccc(N)c(Br)c2)CC1 ; OB(O)C1=CCCC1 ; CC(C)(C)OC(=O)N1CCC(c2ccc(N)c(C3=CCCC3)c2)CC1]

The obtained compound was synthesized in the same manner as in Example 46a, and was separated and purified through column chromatography to obtain 21.6 mg (0.063 mmol) of Compound 47b (Yield: 45%).

1H NMR (400 MHz, CDCl3) δ 7.17 (d, J=7.2 Hz, 1H), 7.04-6.97 (m, 2H), 6.08 (s, 1H), 4.23 (s, 2H), 2.73 (dd, J=20.0, 13.1 Hz, 4H), 2.57 (dd, J=19.8, 7.3 Hz, 4H), 2.05-1.94 (m, 3H), 1.79 (d, J=12.4 Hz, 2H), 1.57 (dd, J=12.6, 3.7 Hz, 2H), 1.48 (s, 9H).

HRMS(ESI+) calculated for C21H31N2NaO2 [M+Na]+: 365.2199, found 365.5803.

### Tert-butyl 4-(4-amino-3-(pyrimidin-5-yl)phenyl)piperidine-1-carboxylate. (48b)

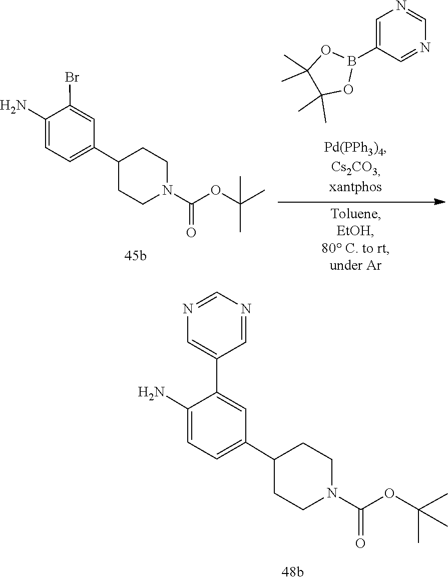 [SMILES: CC(C)(C)OC(=O)N1CCC(c2ccc(N)c(Br)c2)CC1 ; CC1(C)OB(c2cncnc2)OC1(C)C ; CC(C)(C)OC(=O)N1CCC(c2ccc(N)c(-c3cncnc3)c2)CC1]

Compound 48b (26 mg, 52%) was synthesized in the same manner as in Example 46a, and separated and purified by column chromatography.

1H NMR (400 MHz, CDCl3) δ 9.20 (s, 1H), 8.87 (d, J=5.4 Hz, 2H), 7.09 (dd, J=8.3, 2.1 Hz, 1H), 6.92 (d, J=2.1 Hz, 1H), 6.78 (d, J=8.2 Hz, 1H), 4.23 (s, 2H), 2.80 (d, J=12.4 Hz, 2H), 2.59 (ddd, J=12.1, 8.5, 3.5 Hz, 1H), 1.81 (d, J=12.7 Hz, 2H), 1.58 (ddd, J=25.7, 12.7, 4.3 Hz, 4H), 1.47 (d, J=2.0 Hz, 9H).

HRMS (ESI+) calculated for C20H26N4NaO2 [M+Na]+: 377.1948, found 377.5372.

### Tert-butyl 4-(6-Amino-4′-methyl-2′,3′,4′,5′-tetrahydro-[1,1′-biphenyl]-3-yl)piperidine-1-carboxylate. (49b)

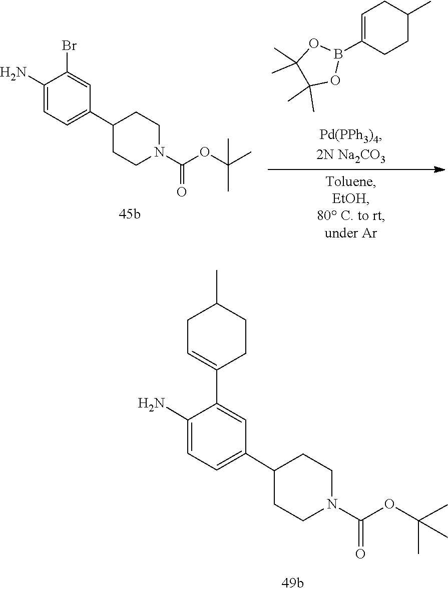

The obtained compound was synthesized in the same manner as in Example 46b, and was separated and purified through column chromatography to obtain 65.1 mg (0.18 mmol) of Compound 49b (yield: 78%).

1H NMR (400 MHz, DMSO-d6) δ 6.77 (dd, J=8.2, 2.1 Hz, 1H), 6.68 (d, J=2.1 Hz, 1H), 6.55 (d, J=8.2 Hz, 1H), 5.57 (d, J=2.5 Hz, 1H), 4.46 (d, J=11.0 Hz, 2H), 2.69 (dd, J=15.1, 13.3 Hz, 2H), 2.47-2.41 (m, 1H), 2.35-2.06 (m, 4H), 1.81-1.62 (m, 6H), 1.40 (s, 9H), 1.38-1.29 (m, 3H), 0.98 (d, J=6.1 Hz, 3H).

HRMS(ESI+) calculated for C23H35N2O2 [M+Na]+: 393.2512, found 393.5262.

### Tert-butyl 4-(6-amino-4′,4′-dimethyl-2′,3′,4′,5′-tetrahydro-[1,1′-biphenyl]-3-yl)piperidine-1-carboxylate. (50b)

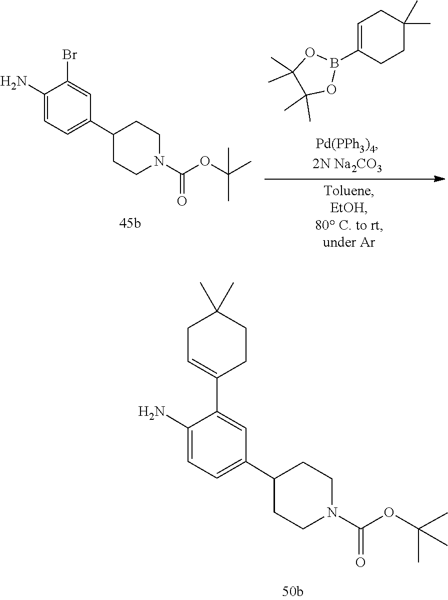

Compound 50b (45.5 mg, 39%) was obtained by synthesis in the same manner as in Example 46b, and separation and purification through column chromatography.

1H NMR (400 MHz, DMSO-d6) δ 6.78 (dd, J=8.2, 2.1 Hz, 1H), 6.69 (d, J=2.1 Hz, 1H), 6.57 (d, J=8.2 Hz, 1H), 5.53 (s, 1H), 4.43 (d, J=11.2 Hz, 2H), 2.74 (s, 2H), 2.53-2.51 (m, 1H), 2.45 (d, J=12.3 Hz, 2H), 2.17 (d, J=2.0 Hz, 2H), 1.93-1.90 (m, 2H), 1.67 (d, J=11.9 Hz, 2H), 1.45 (t, J=6.3 Hz, 2H), 1.40 (s, 9H), 1.39-1.32 (m, 2H), 0.96 (s, 6H).

HRMS (ESI+) calculated for C24H36N2NaO2 [M+Na]+: 407.2669, found 407.5370.

### Tert-butyl 4-(6-amino-4′,4′-dimethyl-2′,3′,4′,5′-tetrahydro-[1,1′-biphenyl]-3-yl)azepane-1-carboxylate. (50c)

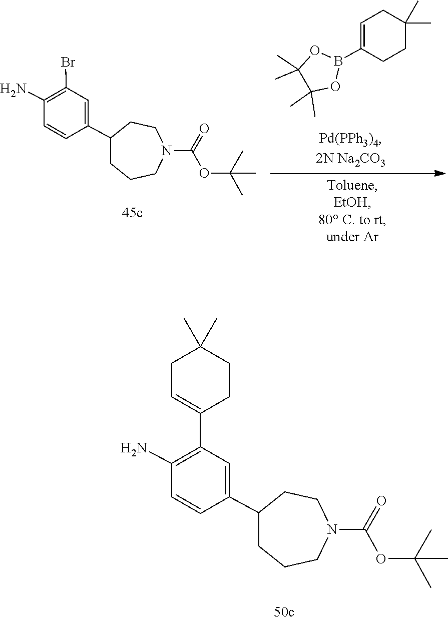

Compound 50c (45.5 mg, 39%) was synthesized in the same manner as in Example 46b, and separated and purified by column chromatography.

1H NMR (400 MHz, DMSO-d6) δ 6.73 (dd, J=8.1, 2.1 Hz, 1H), 6.64 (d, J=1.8 Hz, 1H), 6.55 (d, J=8.1 Hz, 1H), 5.52 (s, 1H), 4.40 (d, J=10.2 Hz, 2H), 3.53 (dd, J=18.4, 14.0 Hz, 1H), 3.44-3.34 (m, 2H), 3.19-3.12 (m, 1H), 2.41 (d, J=10.5 Hz, 1H), 2.16 (d, J=1.9 Hz, 2H), 1.92 (d, J=3.4 Hz, 2H), 1.80 (s, 2H), 1.73 (s, 1H), 1.65-1.49 (m, 3H), 1.45 (t, J=6.4 Hz, 2H), 1.41 (d, J=2.0 Hz, 9H), 0.96 (s, 6H).

HRMS(ESI+) calculated for C25H38N2NaO2 [M+Na]+: 421.2825, found 421.5102.

### Tert-butyl 4-(4-amino-3-cyclohexylphenyl)piperidine-1-carboxylate. (51b)

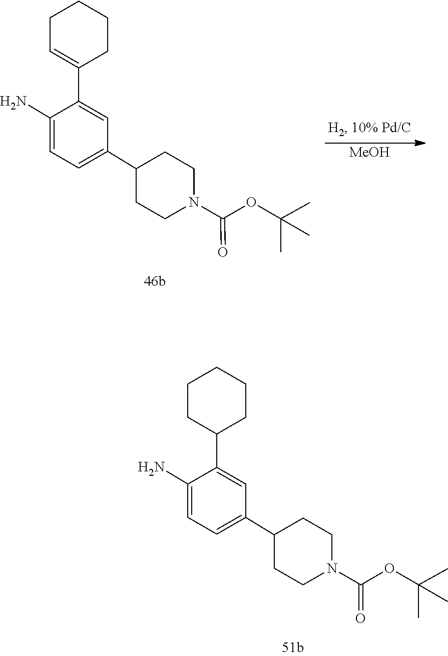 [SMILES: "MeOH" ; CC(C)(C)OC(=O)N1CCC(c2ccc(N)c(C3=CCCCC3)c2)CC1 ; CC(C)(C)OC(=O)N1CCC(c2ccc(N)c(C3CCCCC3)c2)CC1]

Compound 46b (170 mg, 0.47 mmol) obtained above and Pd/C (17 mg) were added and dissolved in MeOH (4.7 mL), and then vacuum was taken and substituted with hydrogen gas, followed by stirring at room temperature for 1 hour. After the completion of the reaction was confirmed, the reaction mixture was filtered through cellite and concentrated to give Compound 51b (110 mg, 70%).

1H NMR (400 MHz, DMSO-d6) δ 6.80 (d, J=1.9 Hz, 1H), 6.71 (dd, J=8.1, 2.0 Hz, 1H), 6.52 (d, J=8.1 Hz, 1H), 4.60 (s, 2H), 4.03 (d, J=11.4 Hz, 2H), 2.74 (s, 2H), 2.53 (dd, J=8.7, 7.0 Hz, 1H), 2.49-2.42 (m, 1H), 1.78-1.63 (m, 7H), 1.41 (s, 9H), 1.39-1.19 (m, 7H). HRMS (ESI+) calculated for C22H34N2NaO2 [M+Na]+: 381.2512, found 381.5348.

### Tert-butyl 4-(4-Aminophenyl)-3-cyclopentylpiperidine-1-carboxylate (52b)

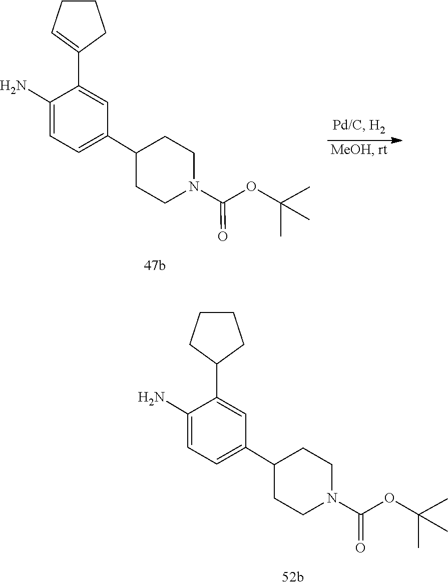 [SMILES: CC(C)(C)OC(=O)N1CCC(c2ccc(N)c(C3=CCCC3)c2)CC1 ; "MeOH, rt" ; CC(C)(C)OC(=O)N1CCC(c2ccc(N)c(C3CCCC3)c2)CC1]

A compound 52b (80.9 mg, 79%) was obtained by the same method as above without further purification.

1H NMR (400 MHz, CDCl3) δ 6.97 (d, J=2.0 Hz, 1H), 6.85 (dd, J=8.1, 2.1 Hz, 1H), 6.63 (d, J=8.0 Hz, 1H), 4.20 (s, 2H), 2.95 (dd, J=16.0, 7.5 Hz, 1H), 2.77 (t, J=12.2 Hz, 2H), 2.52 (tt, J=12.1, 3.5 Hz, 1H), 2.07-1.98 (m, 2H), 1.79 (dd, J=7.4, 6.2 Hz, 2H), 1.66-1.57 (m, 10H), 1.48 (s, 9H).

HRMS(ESI+) calculated for C21H33N202 [M+Na]+:367.2356, found 367.5971

### Tert-butyl 3-(6-(5-methylisoxazole-3-carboxamido)-2′,3′,4′,5′-tetrahydro-[1,1′-biphenyl]-3-yl)pyrrolidine-1-carboxylate. (53a)

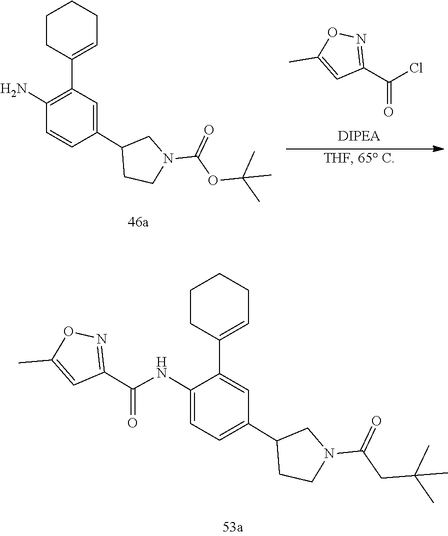

Compound 46a (33 mg, 0.096 mmol) was dissolved in Tetrahydrofuran (0.48 mL), DIPEA (18.8 μL, 0.1056 mmol) was added thereto, and 5-methylisoxazole-3-carbonyl chloride (14 mg, 0.096 mmol) was added dropwise thereto, and the result was stirred at 65° C. for 2 hours. After confirming the completion of the reaction, the reaction mixture was extracted with Ethyl acetate and washed with water and brine. After drying with Anhydrous Na2SO4, the mixture was concentrated. Separation and purification were performed by column chromatography, and compound 53a (31 mg, 71%) was obtained.

1H NMR (400 MHz, DMSO-d6) δ 9.59 (s, 1H), 7.68 (d, J=8.3 Hz, 1H), 7.20 (dd, J=8.2, 1.8 Hz, 1H), 7.12 (s, 1H), 6.87 (s, 1H), 6.74 (d, J=8.3 Hz, 1H), 6.63 (d, J=0.8 Hz, 1H), 5.69 (s, 1H), 5.17 (s, 1H), 3.68 (dd, J=10.2, 7.5 Hz, 1H), 3.45 (dd, J=14.2, 5.7 Hz, 1H), 3.35 (dd, J=3.6, 2.2 Hz, 1H), 3.27 (t, J=6.6 Hz, 1H), 3.16 (t, J=9.8 Hz, 1H), 2.49 (s, 3H), 2.20 (s, 3H), 2.13-2.06 (m, 2H), 1.94 (dd, J=18.2, 8.0 Hz, 1H), 1.72-1.57 (m, 4H), 1.41 (d, J=4.5 Hz, 9H).

HRMS(ESI+) calculated for C26H33N3NaO4 [M+Na]+: 474.2369, found 474.5926.

### Tert-butyl 4-(6-(5-methylisoxazole-3-carboxamido)-2′,3′,4′,5′-tetrahydro-[1,1′-biphenyl]-3-yl)piperidine-1-carboxylate. (53b)

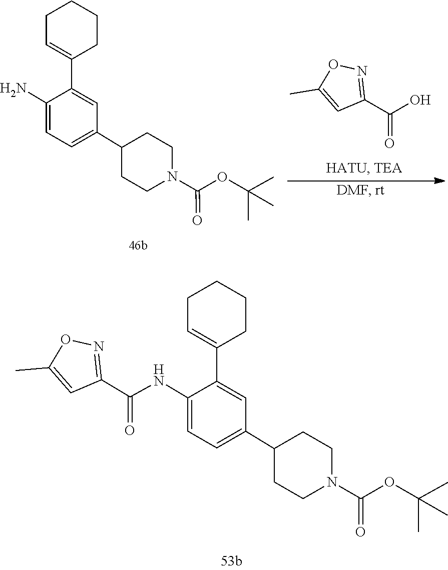

5-methylisoxazole-3-carboxylic acid (80 mg, 0.63 mmol) and HATU (287 mg, 0.756 mmol) were dissolved in DMF (2 mL), Triethylamine (143 μL, 1.05 mmol) was added thereto, and the mixture was stirred at room temperature for 2 hours. Compound 46b (152 mg, 0.42 mmol) was dissolved in DMF (2 mL), and the stirred mixture was added dropwise thereto, followed by stirring for 1 hour. After confirming the completion of the reaction, the reaction mixture was extracted with Ethyl acetate and washed with water and brine. It was dried over Anhydrous Na2SO4, concentrated, and separated and purified using column chromatography to obtain 164.7 mg (84%) of Compound 53b.

1H NMR (400 MHz, DMSO-d6) δ 9.57 (s, 1H), 7.66 (d, J=8.2 Hz, 1H), 7.15 (dd, J=8.4, 1.7 Hz, 1H), 7.07 (d, J=1.7 Hz, 1H), 6.65 (d, J=16.5 Hz, 1H), 5.68 (s, 1H), 4.05 (dd, J=16.4, 9.4 Hz, 2H), 2.75 (d, J=31.1 Hz, 2H), 2.67 (t, J=12.0 Hz, 1H), 2.49 (s, 3H), 2.21 (s, 2H), 2.09 (s, 2H), 1.75 (d, J=12.0 Hz, 2H), 1.70-1.64 (m, 2H), 1.61 (d, J=4.1 Hz, 2H), 1.48 (dd, J=12.6, 4.0 Hz, 2H), 1.41 (s, 9H).

HRMS (ESI+) calculated for C27H35N3NaO4 [M+Na]+:488.2520, found 488.4209.

### Tert-butyl 4-(6-(5-methylisoxazole-3-carboxamido)-2′,3′,4′,5′-tetrahydro-[1,1′-biphenyl]-3-yl)azepane-1-carboxylate. (53c)

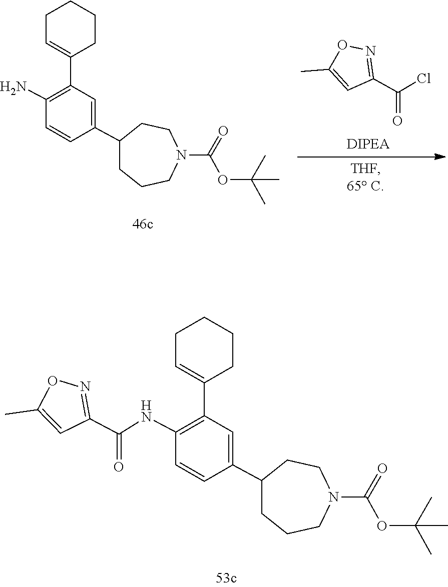

Compound 53c (39 mg, 50%) was synthesized in the same manner as in Example 53a, and separated and purified by column chromatography.

1H NMR (400 MHz, DMSO-d6) δ 9.55 (d, J=5.1 Hz, 1H), 7.63 (d, J=8.2 Hz, 1H), 7.12-7.08 (m, 1H), 7.02 (d, J=2.0 Hz, 1H), 6.62 (d, J=0.9 Hz, 1H), 5.68 (s, 1H), 3.55 (d, J=17.4 Hz, 1H), 3.40 (d, J=15.3 Hz, 2H), 3.22 (s, 1H), 2.62 (s, 1H), 2.49-2.48 (m, 3H), 2.20 (s, 2H), 2.09 (d, J=1.7 Hz, 2H), 1.86 (s, 2H), 1.72 (s, 2H), 1.67 (d, J=5.9 Hz, 3H), 1.61 (t, J=8.7 Hz, 3H), 1.42 (d, J=2.1 Hz, 9H).

HRMS(ESI+) calculated for C28H37N3NaO4 [M+Na]+: 502.2682, found 502.8974.

### tert-butyl 4-(3-(Cyclopent-1-en-1-yl)-4-(5-methylisoxazole-3-carboxamido) phenyl) piperidine-1-carboxylate (54b)

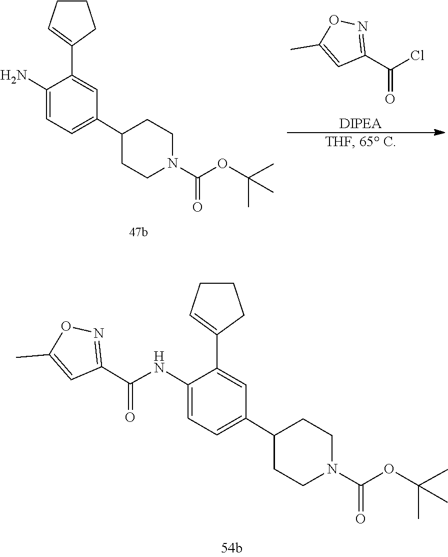

The obtained compound was synthesized in the same manner as in Example 53a, and was separated and purified through column chromatography to obtain Compound 54b (6.7 mg, 0.0015 mmol, 35%).

1H NMR (400 MHz, CDCl3) δ 8.96 (s, 1H), 8.28 (d, J=8.4 Hz, 1H), 7.11 (dd, J=8.4, 2.1 Hz, 1H), 7.07 (d, J=2.0 Hz, 1H), 6.50 (s, 1H), 6.00-5.95 (m, 1H), 2.79 (s, 2H), 2.69 (d, J=1.9 Hz, 1H), 2.62 (d, J=4.8 Hz, 2H), 2.52 (d, J=2.2 Hz, 1H), 2.49 (s, 3H), 2.07 (d, J=3.3 Hz, 1H), 2.04 (s, 1H), 2.03 (d, J=3.0 Hz, 1H), 1.80-1.78 (m, 2H), 1.61 (s, 2H), 1.59 (s, 2H), 1.47 (s, 9H).

HRMS(ESI+) calculated for C26H33N3NaO4 [M+Na]+: 474.2363, found 474.6654.

### Tert-butyl 4-(4-(5-methylisoxazole-3-carboxamido)-3-(pyrimidin-5-yl)phenyl)piperidine-1-carboxylate. (55b)

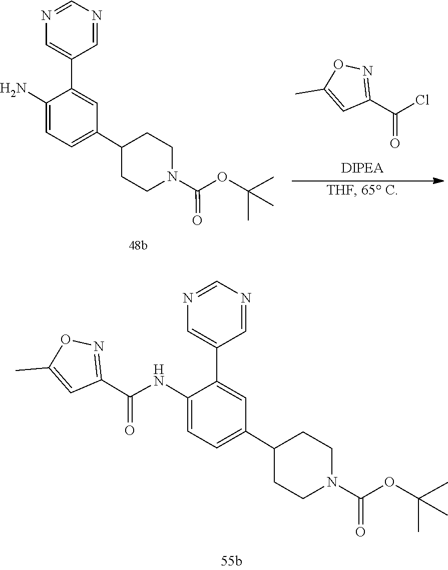

Compound 55b (25 mg, 73%) was synthesized in the same manner as in Example 53a, and separated and purified by column chromatography.

1H NMR (400 MHz, Acetone) δ 9.29 (s, 1H), 9.10 (s, 1H), 8.86 (d, J=0.8 Hz, 2H), 7.76 (dd, J=8.2, 5.8 Hz, 1H), 7.46-7.43 (m, 1H), 7.42 (d, J=2.1 Hz, 1H), 6.43 (d, J=0.9 Hz, 1H), 4.23 (d, J=11.7 Hz, 2H), 2.93-2.83 (m, 3H), 2.47 (d, J=0.8 Hz, 3H), 1.89 (dd, J=6.8, 4.6 Hz, 2H), 1.66 (dt, J=13.2, 8.6 Hz, 2H), 1.45 (s, 9H).

HRMS (ESI+) calculated for C25H29N5NaO4 [M+Na]+: 486.2112, found 486.6564.

### Tert-butyl 4-(4′-Methyl-6-(5-methylisoxazole-3-carboxamido)-2,3,4,5′-tetrahydro [1,1′-biphenyl]-3-yl)piperidine-1-carboxylate (56b)

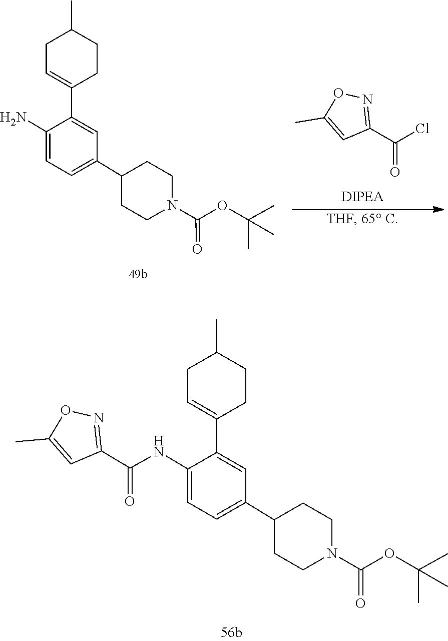

Compound 56b (41.1 mg, 0.086 mmol, 78%) was obtained in the same manner as in Example 53a without further purification.

1H NMR (400 MHz, DMSO-d6) δ 9.56 (s, 1H), 7.65 (d, J=8.3 Hz, 1H), 7.15 (dd, J=8.3, 2.0 Hz, 1H), 7.08 (d, J=2.0 Hz, 1H), 6.62 (d, J=0.8 Hz, 1H), 5.65 (s, 1H), 4.12-4.00 (m, 2H), 2.76 (d, J=25.2 Hz, 2H), 2.67 (t, J=10.0 Hz, 1H), 2.49 (s, 3H), 2.25-2.15 (m, 2H), 1.73 (t, J=12.3 Hz, 6H), 1.57-1.45 (m, 2H), 1.41 (s, 9H), 1.32 (d, J=7.0 Hz, 1H), 0.97 (d, J=6.1 Hz, 3H).

HRMS(ESI+) calculated for C28H37N3NaO4 [M+Na]+: 502.2676, found 502.8974.

### Tert-butyl 4-(4′,4′-dimethyl-6-(5-methylisoxazole-3-carboxamido)-2′,3′,4′,5′-tetrahydro-[1,1′-biphenyl]-3-yl)piperidine-1-carboxylate. (57b)

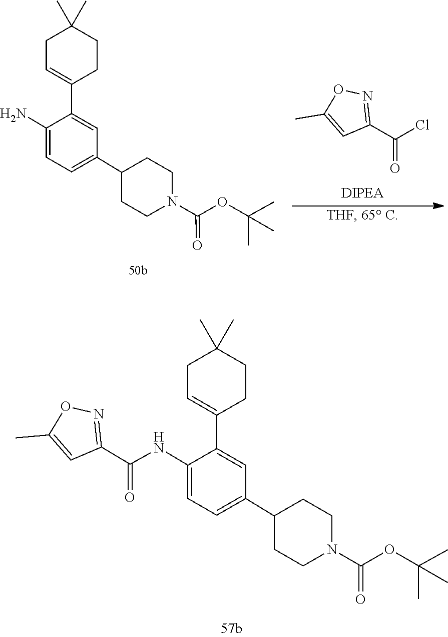

Compound 57b (55.8 mg, 95%) was synthesized in the same manner as in Example 53a, and separated and purified by column chromatography.

1H NMR (400 MHz, DMSO-d6) δ 9.61 (s, 1H), 7.65 (d, J=8.2 Hz, 1H), 7.17 (d, J=8.3 Hz, 1H), 7.11 (d, J=1.9 Hz, 1H), 6.62 (d, J=0.9 Hz, 1H), 5.62 (s, 1H), 4.08 (s, 2H), 2.82 (s, 2H), 2.71 (d, J=8.9 Hz, 1H), 2.50 (s, 3H), 2.26 (s, 2H), 1.91 (s, 2H), 1.76 (s, 2H), 1.56-1.45 (m, 4H), 1.43 (s, 9H), 0.96 (s, 6H).

HRMS (ESI+) calculated for C29H40N3O4 [M+H]+: 494.3013, found 494.5420.

### tert-butyl 4-(4′,4′-dimethyl-6-(5-methylisoxazole-3-carboxamido)-2′,3′,4′,5′-tetrahydro-[1,1′-biphenyl]-3-yl)azepane-1-carboxylate. (57c)

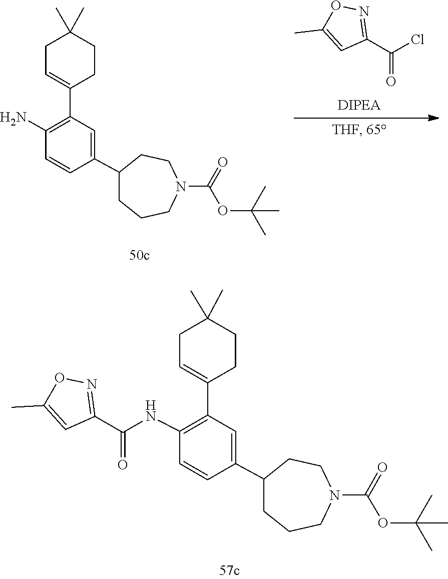

Compound 57c (26 mg, 73%) was synthesized in the same manner as in Example 53a, and separated and purified by column chromatography.

1H NMR (400 MHz, DMSO-d6) δ 9.57 (d, J=5.3 Hz, 1H), 7.66-7.57 (m, 1H), 7.10 (d, J=8.3 Hz, 1H), 7.03 (d, J=1.9 Hz, 1H), 6.60 (d, J=0.9 Hz, 1H), 5.60 (d, J=1.8 Hz, 1H), 3.61-3.53 (m, 1H), 3.46-3.33 (m, 2H), 3.21 (dd, J=13.9, 10.2 Hz, 1H), 2.65-2.59 (m, 1H), 2.48 (s, 3H), 2.23 (s, 2H), 1.88 (s, 2H), 1.87-1.68 (m, 4H), 1.67-1.56 (m, 2H), 1.43 (t, J=5.0 Hz, 11H), 0.94 (s, 6H).

HRMS(ESI+) calculated for C30H41N3NaO4 [M+Na]+: 530.2995, found 530.3815.

### Tert-butyl 4-(3-cyclohexyl-4-(5-methylisoxazole-3-carboxamido)phenyl)piperidine-1-carboxylate (58b)

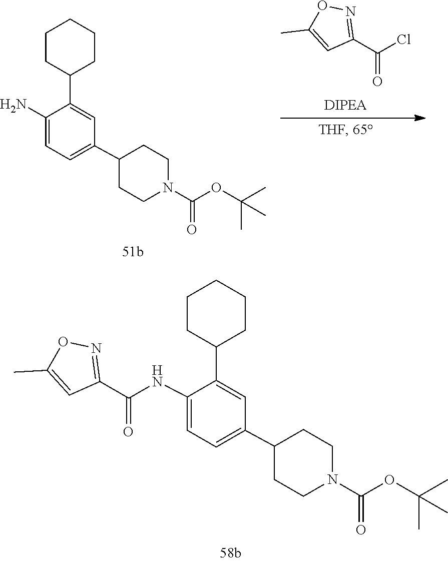

Compound 58b (72 mg, 66%) was synthesized in the same manner as in Example 53a, and separated and purified by column chromatography.

1H NMR (400 MHz, CDCl3) δ 8.46 (s, 1H), 7.81 (d, J=8.2 Hz, 1H), 7.12 (d, J=2.0 Hz, 1H), 7.08 (dd, J=8.3, 2.0 Hz, 1H), 6.54 (d, J=0.9 Hz, 1H), 4.25 (d, J=13.2 Hz, 2H), 2.80 (td, J=13.1, 2.4 Hz, 2H), 2.63 (ddd, J=12.1, 7.7, 3.3 Hz, 2H), 2.52 (d, J=0.8 Hz, 3H), 1.82 (dd, J=32.5, 12.3 Hz, 7H), 1.67-1.59 (m, 3H), 1.49 (s, 9H), 1.47-1.41 (m, 4H).

HRMS (ESI+) calculated for C27H37N3NaO4 [M+Na]+: 490.2676, found 490.6176.

### Tert-butyl 4-(3-Cyclopentyl-4-(5-methylisoxazole-3-carboxamido)phenyl) piperidine-1-carboxylate (59b)

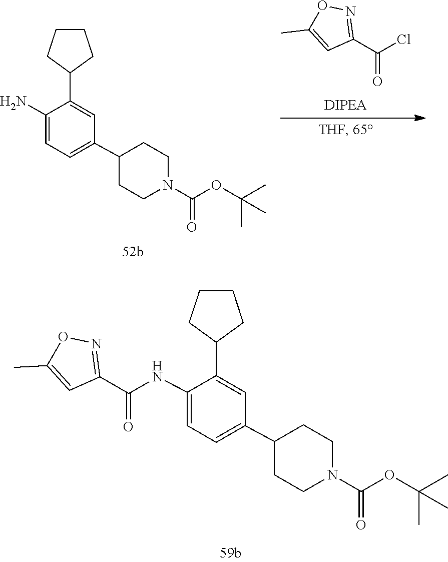

The procedure was performed in the same manner as in Example 53a, and column chromatography (developing solvent EA:HEX=1:7) was performed thereon to obtain Compound 59b (39.4 mg, 0.087 mmol, 36%).

1H NMR (400 MHz, CDCl3) δ 8.51 (s, 1H), 7.89 (d, J=8.2 Hz, 1H), 7.18 (d, J=1.9 Hz, 1H), 7.12 (dd, J=8.3, 2.0 Hz, 1H), 6.57 (d, J=0.9 Hz, 1H), 4.27 (t, J=10.7 Hz, 2H), 3.16 (dd, J=16.5, 7.8 Hz, 1H), 2.91-2.78 (m, 2H), 2.67 (t, J=12.0 Hz, 1H), 2.55 (t, J=1.9 Hz, 3H), 2.12 (t, J=9.3 Hz, 2H), 1.85 (d, J=9.6 Hz, 4H), 1.79-1.72 (m, 2H), 1.71-1.62 (m, 4H), 1.51 (d, J=5.6 Hz, 9H); HRMS(ESI+) calculated for C26H35N3NaO4 [M+Na]+: 476.2520, found 476.8973.

### 5-methyl-N-(5-(pyrrolidin-3-yl)-2′,3′,4′,5′-tetrahydro-[1,1′-biphenyl]-2-yl)isoxazole-3-carboxamide. (60a)

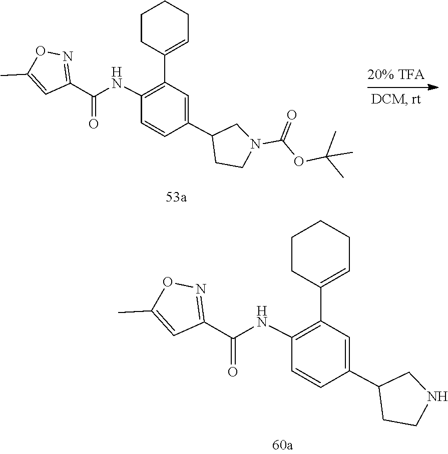

Compound 53a (31 mg, 0.68 mmol) obtained above was dissolved in DCM (0.544 mL), and Trifluoroacetic acid (0.136 mL) was added dropwise and stirred at room temperature for 30 minutes. After the completion of the reaction was confirmed, the reaction mixture was washed with NaHCO₃, 1M NaOH, water and brine to obtain 6 mg (0.017 mmol, 25%) of Compound 60a.

1H NMR (400 MHz, CDCl3) δ 8.99 (s, 1H), 8.28 (dd, J=8.4, 1.4 Hz, 1H), 7.21-7.17 (m, 1H), 7.06 (d, J=2.1 Hz, 1H), 6.51-6.49 (m, 1H), 5.80 (dd, J=4.6, 2.7 Hz, 1H), 3.32 (dd, J=8.7, 5.7 Hz, 1H), 3.13 (dd, J=17.1, 8.4 Hz, 1H), 2.93-2.78 (m, 2H), 2.67-2.61 (m, 1H), 2.51-2.47 (m, 3H), 2.35-2.27 (m, 1H), 2.22 (dd, J=11.6, 5.7 Hz, 4H), 2.05-2.00 (m, 1H), 1.92-1.83 (m, 1H), 1.81-1.76 (m, 2H), 1.75-1.72 (m, 2H).

HRMS(ESI+) calculated for C21H26N3O2 [M+H]+: 352.2020, found 352.9029.

### 5-methyl-N-(5-(piperidin-4-yl)-2′,3′,4′,5′-tetrahydro-[1,1′-biphenyl]-2-yl)isoxazole-3-carboxamide. (60b)

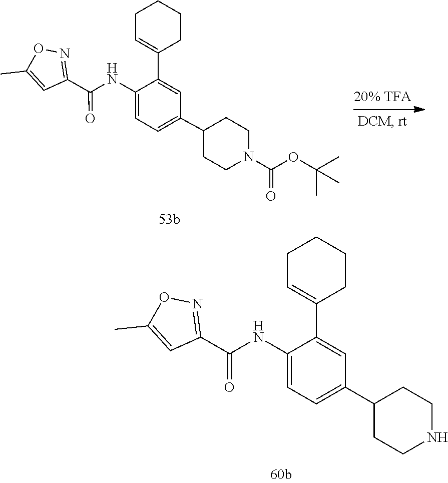

A compound 60b (83 mg, 0.227 mmol, 85%) was prepared by the same method as above without further purification.

1H NMR (400 MHz, DMSO-d6) δ 9.57 (s, 1H), 7.65 (d, J=8.2 Hz, 1H), 7.13 (dd, J=8.3, 2.1 Hz, 1H), 7.04 (d, J=2.0 Hz, 1H), 6.63 (d, J=0.8 Hz, 1H), 5.68 (s, 1H), 3.06 (d, J=11.9 Hz, 2H), 2.68-2.56 (m, 3H), 2.49-2.49 (m, 3H), 2.20 (s, 2H), 2.09 (s, 2H), 2.05-1.96 (m, 1H), 1.70 (dd, J=12.3, 9.3 Hz, 4H), 1.61 (dd, J=7.4, 3.7 Hz, 2H), 1.56-1.47 (m, 2H).

HRMS (ESI+) calculated for C22H28N3O2 [M+H]+:366.2176, found 366.3726.

### N-(5-(azepan-4-yl)-2′,3′,4′,5′-tetrahydro-[1,1′-biphenyl]-2-yl)-5-methylisoxazole-3-carboxamide. (60c)

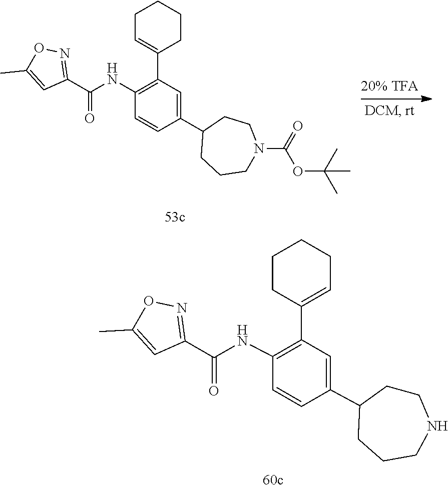

A compound 60c (30 mg, 0.079 mmol, 100%) was obtained by the same method as above without further purification.

1H NMR (400 MHz, DMSO-d6) δ 9.54 (s, 1H), 7.64 (t, J=9.8 Hz, 1H), 7.12 (d, J=8.2 Hz, 1H), 7.04 (d, J=1.8 Hz, 1H), 6.62 (s, 1H), 5.68 (s, 1H), 2.93-2.88 (m, 1H), 2.84-2.68 (m, 3H), 2.49 (s, 3H), 2.47 (s, 1H), 2.20 (s, 2H), 2.09 (s, 2H), 1.95 (d, J=13.8 Hz, 1H), 1.82 (dd, J=22.8, 6.2 Hz, 2H), 1.77 (s, 2H), 1.65 (d, J=13.6 Hz, 3H), 1.61 (d, J=3.7 Hz, 3H).

HRMS(ESI+) calculated for C23H30N3O2+[M+H]+: 380.2333, found 380.8505.

### N-(2-(Cyclopent-1-en-1-yl)-4-(piperidin-4-yl)phenyl)-5-methylisoxazole-3-carboxamide (61b)

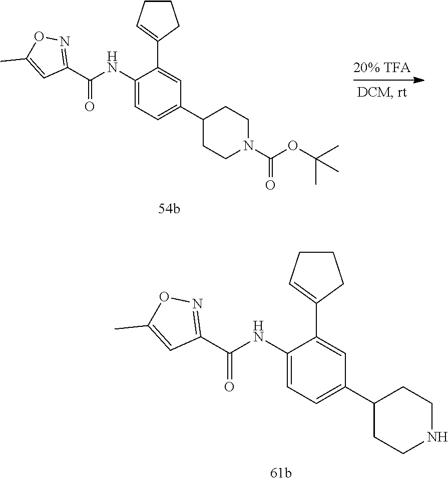

A compound 61b (14.5 mg, 0.041 mmol, 100%) was obtained without further purification by the same method as above.

1H NMR (400 MHz, DMSO-d6) δ 9.83 (s, 1H), 7.51 (d, J=8.0 Hz, 1H), 7.26-7.10 (m, 2H), 6.63 (s, 1H), 6.01 (s, 1H), 3.08 (s, 2H), 2.65 (d, J=8.1 Hz, 5H), 2.50 (s, 3H), 2.16 (s, 1H), 2.03 (s, 1H), 1.97-1.86 (m, 3H), 1.73 (s, 2H), 1.57 (d, J=11.2 Hz, 2H).

HRMS(ESI+) calculated for C21H26N3O2 [M+H]+: 352.2020, found 352.5787.

### 5-methyl-N-(4-(piperidin-4-yl)-2-(pyrimidin-5-yl)phenyl)isoxazole-3-carboxamide. (62b)

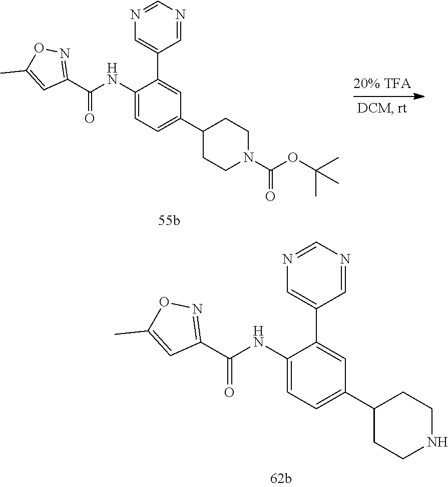

A compound 62b (4 mg, 0.011 mmol, 25%) was obtained by the same method as above without further purification.

1H NMR (400 MHz, CDCl3) δ 9.25 (d, J=6.2 Hz, 1H), 8.81 (s, 2H), 8.30 (s, 1H), 7.98 (d, J=8.4 Hz, 1H), 7.38 (dd, J=8.4, 2.0 Hz, 1H), 7.17 (d, J=2.0 Hz, 1H), 6.45 (d, J=0.8 Hz, 1H), 3.26 (d, J=12.2 Hz, 2H), 2.79 (dd, J=12.3, 10.0 Hz, 2H), 2.71 (t, J=12.1 Hz, 1H), 2.47 (d, J=0.5 Hz, 3H), 1.90 (d, J=12.9 Hz, 2H), 1.77-1.67 (m, 3H).

HRMS (ESI+) calculated for C20H22N5O2 [M+H]+: 364.1768, found 364.5718.

### 5-Methyl-N-(4′-methyl-5-(piperidin-4-yl)-2′,3′,4′,5′-tetrahydro-[1,1′-biphenyl]-2-yl)isoxazole-3-carboxamide (63b)

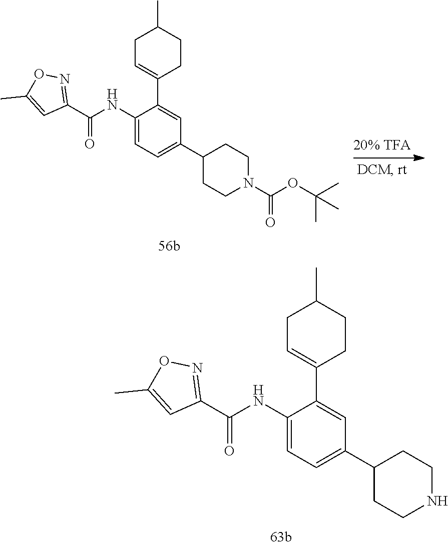

The reaction mixture was synthesized in the same manner as above, and separated and purified through column chromatography to obtain 3.3 mg (0.0087 mmol, 11%) of Compound 63b.

1H NMR (400 MHz, DMSO-d6) δ 9.63 (s, 1H), 7.69 (d, J=8.2 Hz, 1H), 7.16 (d, J=10.1 Hz, 1H), 7.07 (d, J=2.0 Hz, 1H), 6.64 (s, 1H), 5.68 (s, 1H), 2.90 (t, J=11.5 Hz, 2H), 2.79 (s, 1H), 2.53 (s, 3H), 2.22 (d, J=16.3 Hz, 2H), 1.94 (dd, J=27.9, 15.4 Hz, 4H), 1.75 (s, 5H), 1.48 (s, 2H), 1.37-1.32 (m, 1H), 1.00 (d, J=6.1 Hz, 3H).

HRMS(ESI+) calculated for C23H30N3O2 [M+H]+: 380.2333, found 380.8505.

### N-(4′,4′-dimethyl-5-(piperidin-4-yl)-2′,3′,4′,5′-tetrahydro-[1,1′-biphenyl]-2-yl)-5-methylisoxazole-3-carboxamide. (64b)

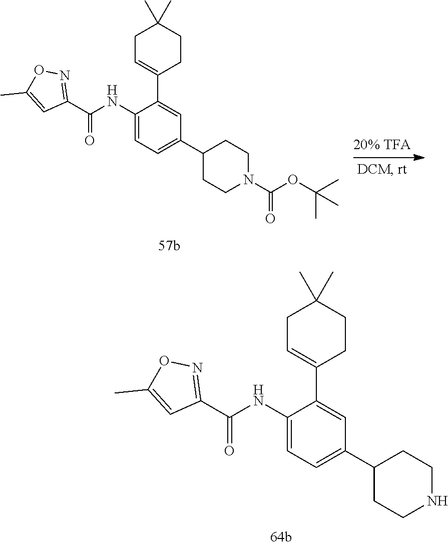

A compound 64b (45.3 mg, 0.115 mmol, 100%) was obtained by the same method as above without further purification.

1H NMR (400 MHz, DMSO-d6) δ 9.59 (s, 1H), 7.63 (dd, J=11.7, 8.3 Hz, 1H), 7.15-7.12 (m, 1H), 7.06 (d, J=1.9 Hz, 1H), 6.60 (d, J=0.9 Hz, 1H), 5.60 (s, 1H), 3.05 (d, J=12.1 Hz, 2H), 2.60 (t, J=12.1 Hz, 3H), 2.49 (s, 3H), 2.24 (s, 2H), 1.89 (s, 2H), 1.70 (d, J=12.4 Hz, 2H), 1.52 (dd, J=12.5, 3.7 Hz, 2H), 1.44 (t, J=6.2 Hz, 2H), 1.23 (s, 1H), 0.94 (s, 6H).

HRMS (ESI+) calculated for C24H32N3O2 [M+H]+: 394.2489, found 394.8961.

### N-(5-(azepan-4-yl)-4′,4′-dimethyl-2′,3′,4′,5′-tetrahydro-[1,1′-biphenyl]-2-yl)-5-methylisoxazole-3-carboxamide. (64c)

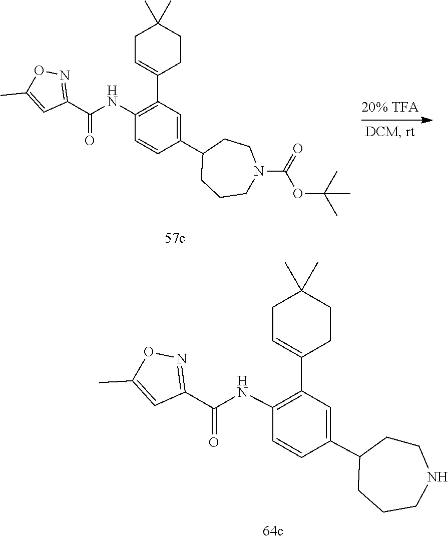

A compound 64c (29.4 mg, 0.072 mmol, 92%) was obtained by the same method as above without further purification.

1H NMR (400 MHz, DMSO-d6) δ 9.56 (s, 1H), 7.64-7.58 (m, 1H), 7.12 (d, J=8.3 Hz, 1H), 7.05 (d, J=1.8 Hz, 1H), 6.60 (s, 1H), 5.59 (s, 1H), 2.90 (d, J=11.2 Hz, 1H), 2.84-2.69 (m, 3H), 2.49 (s, 3H), 2.48 (s, 1H), 2.23 (s, 2H), 1.95 (d, J=14.2 Hz, 1H), 1.88 (s, 2H), 1.81-1.57 (m, 6H), 1.43 (t, J=6.3 Hz, 2H), 0.93 (d, J=4.8 Hz, 6H).

HRMS(ESI+) calculated for C25H34N3O2 [M+H]+: 408.2646, found 408.3415.

### N-(2-cyclohexyl-4-(piperidin-4-yl)phenyl)-5-methylisoxazole-3-carboxamide. (65b)

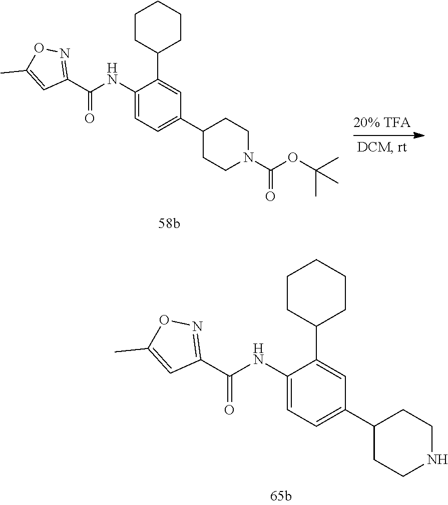

A compound 65b (35 mg, 0.095 mmol, 95%) was prepared by the same method as above without further purification.

1H NMR (400 MHz, DMSO-d6) δ 10.08 (s, 1H), 7.17-7.12 (m, 2H), 7.05 (dd, J=8.1, 1.8 Hz, 1H), 6.62 (d, J=0.8 Hz, 1H), 3.01 (d, J=11.8 Hz, 2H), 2.74-2.66 (m, 1H), 2.57 (t, J=11.1 Hz, 3H), 2.50 (s, 3H), 1.78-1.65 (m, 7H), 1.51 (qd, J=12.3, 3.8 Hz, 2H), 1.29 (dt, J=30.9, 16.5 Hz, 6H).

HRMS (ESI+) calculated for C22H30N3O2 [M+H]+: 368.2333, found 368.4975.

### N-(2-Cyclopentyl-4-(piperidin-4-yl)phenyl)-5-methylisoxazole-3-carboxamide (66b)

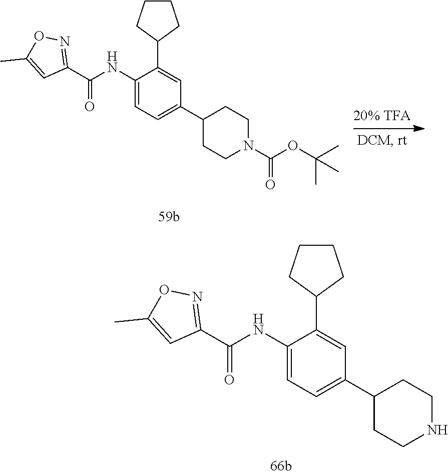

A compound 66b (25 mg, 0.071 mmol, 83%) was obtained without further purification by the same method as above.

1H NMR (400 MHz, DMSO-d6) δ 10.06 (s, 1H), 7.19-7.14 (m=2H), 7.05 (dd, J=8.1, 1.9 Hz, 1H), 6.62 (d, J=0.9 Hz, 1H), 3.20-3.09 (m, 1H), 3.02 (d, J=12.0 Hz, 2H), 2.58 (t, J=11.6 Hz, 3H), 1.93 (t, J=3.9 Hz, 4H), 1.78-1.61 (m, 5H), 1.62-1.43 (m, 7H).

HRMS(ESI+) calculated for C21H28N3O2 [M+H]+: 354.2176, found 354.8837.

### 5-methyl-N-(5-(1-methylpiperidin-4-yl)-2′,3′,4′,5′-tetrahydro-[1,1′-biphenyl]-2-yl)isoxazole-3-carboxamide. (67b)

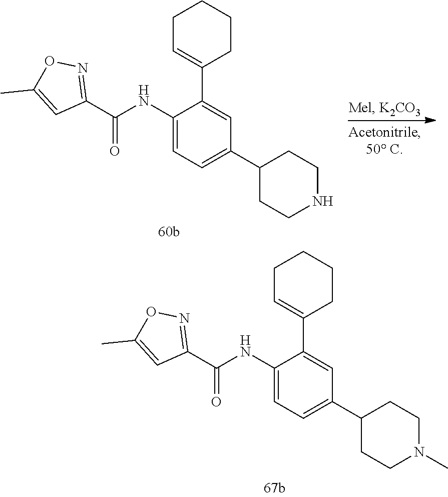

Compound 60b (15 mg, 0.041 mmol) obtained above and Potassium carbonate (5.2 mg, 0.061 mmol) were dissolved in Acetonitrile (1.03 mL), and Iodomethane (3.82 μL, 0.0615 mmol) was stirred at 50° C. for 3 hours. After confirming the completion of the reaction, it was moved to room temperature, filtered with Ethyl acetate, and concentrated. Separation and purification by column chromatography yielded Compound 67b (7.5 mg, 0.0198 mmol, 48%).

1H NMR (400 MHz, DMSO-d6) δ 9.60 (s, 1H), 7.67 (d, J=8.3 Hz, 1H), 7.15 (d, J=8.3 Hz, 1H), 7.07 (s, 1H), 6.63 (d, J=0.8 Hz, 1H), 5.69 (s, 1H), 3.29 (s, 3H), 3.26-3.18 (m, 2H), 2.56 (s, 3H), 2.49-2.49 (m, 3H), 2.19 (d, J=7.1 Hz, 2H), 1.97 (s, 1H), 1.94 (s, 1H), 1.75 (d, J=12.8 Hz, 2H), 1.68 (d, J=5.9 Hz, 2H), 1.61 (d, J=3.9 Hz, 2H), 1.45 (s, 2H).

HRMS (ESI+) calculated for C23H30N3O2 [M+H]+: 380.2333, found 380.1302.

### 5-methyl-N-(5-(1-methylazepan-4-yl)-2′,3′,4′,5′-tetrahydro-[1,1′-biphenyl]-2-yl)isoxazole-3-carboxamide. (67c)

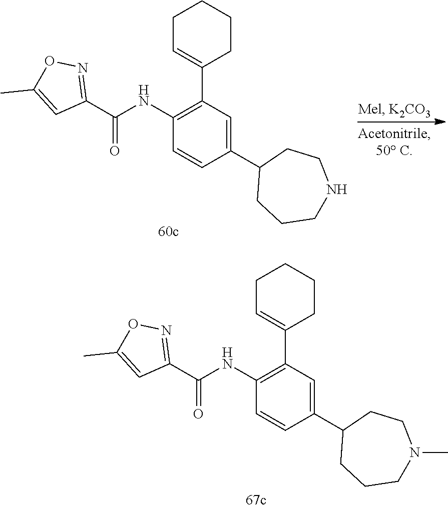

The present invention provides a compound 67c (1.8 mg, 0.0046 mmol, 25%) obtained by synthesis and separation and purification through column chromatography

1H NMR (400 MHz, DMSO-d6) δ 9.58 (s, 1H), 7.65 (d, J=8.2 Hz, 1H), 7.18-7.13 (m, 1H), 7.09 (d, J=1.9 Hz, 1H), 6.62 (s, 1H), 5.69 (s, 1H), 3.17 (d, J=5.0 Hz, 4H), 2.84 (s, 1H), 2.74 (s, 3H), 2.49 (s, 3H), 2.20 (s, 2H), 2.10 (s, 3H), 1.96-1.86 (m, 3H), 1.81 (s, 1H), 1.70 (dd, J=16.4, 12.9 Hz, 3H), 1.61 (d, J=4.0 Hz, 2H).

HRMS(ESI+) calculated for C24H32N3O2 [M+H]+: 394.2489, found 394.7160.

### N-(4,4′-dimethyl-5-(1-methylazepan-4-yl)-2′,3′,4′,5′-tetrahydro-[1,1′-biphenyl]-2-yl)-5-methylisoxazole-3-carboxamide. (68c)

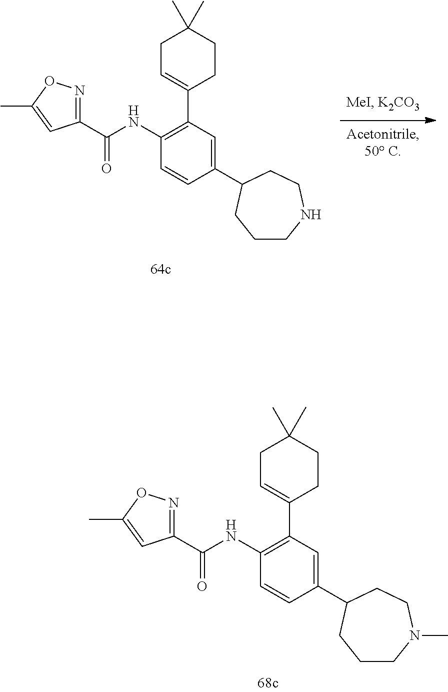

The reaction mixture was synthesized in the same manner as above, and separated and purified through column chromatography to obtain 2.2 mg (0.0052 mmol, 40%) of Compound 68c.

1H NMR (400 MHz, DMSO-d6) δ 9.61 (d, J=2.4 Hz, 1H), 7.64 (t, J=10.0 Hz, 1H), 7.18-7.12 (m, 1H), 7.11-7.05 (m, 1H), 6.60 (d, J=0.9 Hz, 1H), 5.60 (s, 1H), 3.23 (s, 2H), 3.13 (dd, J=21.4, 8.3 Hz, 2H), 2.83 (s, 1H), 2.50-2.49 (m, 3H), 2.49 (s, 3H), 2.24 (s, 2H), 2.01-1.91 (m, 3H), 1.89 (s, 2H), 1.86-1.65 (m, 3H), 1.44 (t, J=6.3 Hz, 2H), 0.94 (s, 6H).

HRMS(ESI+) calculated for C26H36N3O2 [M+H]+: 422.2802, found 422.4825.

### N-(2-cyclohexyl-4-(1-methylpiperidin-4-yl)phenyl)-5-methylisoxazole-3-carboxamide. (69b)

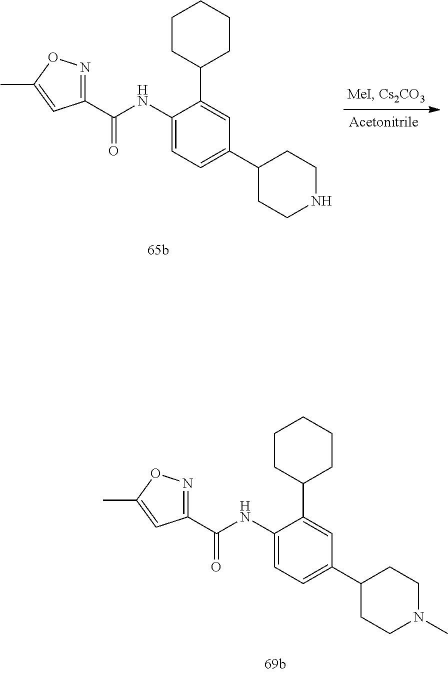

Compound 65b (10 mg, 0.027 mmol) and Cessium carbonate (13 mg, 0.405 mmol) were dissolved in Acetonitrile (0.68 mL), Iodomethane (1.68 μL, 0.027 mmol) was added dropwise, and the resulting mixture was stirred for 7 hours. After confirming the completion of the reaction, the reaction mixture was filtered with MC:MeOH=10:1 and concentrated. Separation and purification by column chromatography yielded Compound 69b (4 mg, 0.010 mmol, 37%).

1H NMR (400 MHz, MeOD) δ 7.34 (d, J=8.1 Hz, 1H), 7.26 (d, J=2.0 Hz, 1H), 7.14 (dd, J=8.2, 2.0 Hz, 1H), 6.54 (d, J=0.9 Hz, 1H), 3.42 (d, J=12.1 Hz, 2H), 2.82 (ddd, J=15.6, 12.0, 8.2 Hz, 4H), 2.74 (s, 3H), 2.52 (d, J=0.8 Hz, 3H), 2.03 (d, J=12.6 Hz, 2H), 1.93 (td, J=12.5, 3.1 Hz, 2H), 1.79 (dd, J=30.6, 11.1 Hz, 5H), 1.52-1.32 (m, 5H).

HRMS (ESI+) calculated for C23H32N3O2 [M+H]+: 382.2489, found 382.2792.

### N-(5-(1-ethylpiperidin-4-yl)-2′,3′,4′,5′-tetrahydro-[1,1′-biphenyl]-2-yl)-5-methylisoxazole-3-carboxamide. (70b)

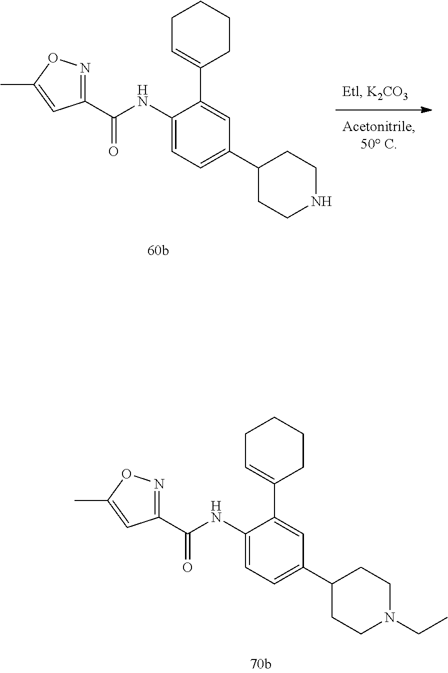 [SMILES: Cc1cc(C(=O)Nc2ccc(C3CCNCC3)cc2C2=CCCCC2)no1 ; CCN1CCC(c2ccc(NC(=O)c3cc(C)on3)c(C3=CCCCC3)c2)CC1]

In the present invention, Compound 60b (15 mg, 0.041 mmol) obtained above and Potassium carbonate (5.2 mg, 0.061 mmol) were added and dissolved in Acetonitrile (1.33 mL), and then Iodoethane (4.28 μL, 0.532 mmol) was added dropwise at 50° C., followed by stirring for 12 hours. After confirming the completion of the reaction, it was moved to room temperature, filtered with Ethyl acetate, and concentrated. separation and purification using column chromatography to give compound 70b (12 mg, 0.030 mmol, 73%) 1H NMR (400 MHz, DMSO-d6) δ 9.62 (s, 1H), 7.72-7.66 (m, 1H), 7.16 (d, J=8.5 Hz, 1H), 7.08 (s, 1H), 6.63 (d, J=0.8 Hz, 1H), 5.70 (s, 1H), 3.48 (d, J=23.3 Hz, 2H), 3.04 (s, 2H), 2.77 (s, 2H), 2.52 (d, J=5.2 Hz, 1H), 2.49 (s, 3H), 2.21 (s, 2H), 2.14-2.09 (m, 2H), 2.00-1.94 (m, 2H), 1.82 (s, 2H), 1.71-1.65 (m, 2H), 1.61 (dd, J=7.2, 3.2 Hz, 2H), 1.20 (d, J=5.0 Hz, 3H).

HRMS (ESI+) calculated for C24H32N3O2 [M+H]+: 394.2489, found 394.1038.

### N-(2-(Cyclopent-1-en-1-yl)-4-(1-ethylpiperidin-4-yl)phenyl)-5-methylisoxazole-3-carboxamide. (71 b)

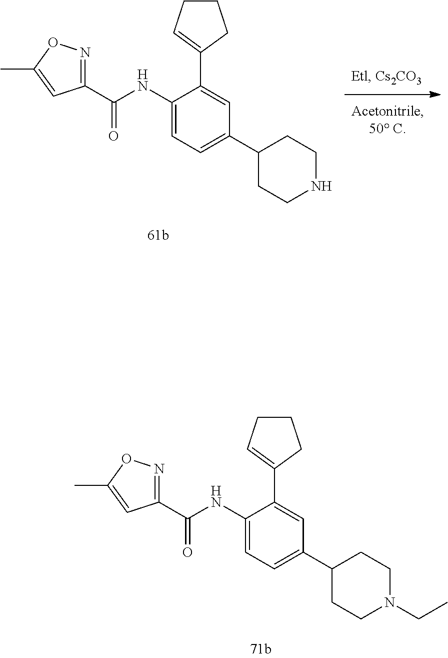 [SMILES: Cc1cc(C(=O)Nc2ccc(C3CCNCC3)cc2C2=CCCC2)no1 ; CCN1CCC(c2ccc(NC(=O)c3cc(C)on3)c(C3=CCCC3)c2)CC1]

Compound 61b (5 mg, 0.014 mmol) was dissolved in Acetonitrile (0.35 mL), and Cessium carbonate (6.84 mg, 0.021 mmol) was added thereto. Iodoethane (2.8 μL, 0.033 mmol) was added dropwise at 50° C. and stirred for 2 hours. Cs2CO3 was removed by filtration under reduced pressure, and Prep TLC (developing solvent: MC:MeOH=10:1) was performed to obtain Compound 71b (2.2 mg, 0.0058 mmol, 41%).

1H NMR (400 MHz, DMSO-d6) δ 9.83 (s, 1H), 7.51 (d, J=8.2 Hz, 1H), 7.22-7.19 (m, 1H), 7.18-7.13 (m, 1H), 6.63 (s, 1H), 6.01 (s, 1H), 3.09 (s, 3H), 2.65 (dd, J=11.1, 3.7 Hz, 2H), 2.50 (s, 3H), 2.17 (d, J=7.1 Hz, 2H), 1.99-1.86 (m, 4H), 1.81 (d, J=14.0 Hz, 2H), 1.74-1.67 (m, 2H), 1.45 (s, 2H), 1.07 (dd, J=9.5, 4.7 Hz, 3H).

HRMS(ESI+) calculated for C23H30N3O2 [M+H]+: 380.2333, found 380.5984.

### N-(5-(1-Ethylpiperidin-4-yl)-4′-methyl-2′,3′,4′,5′-tetrahydro-[1,1′-biphenyl]-2-yl)-5-methylisoxazole-3-carboxamide (72b)

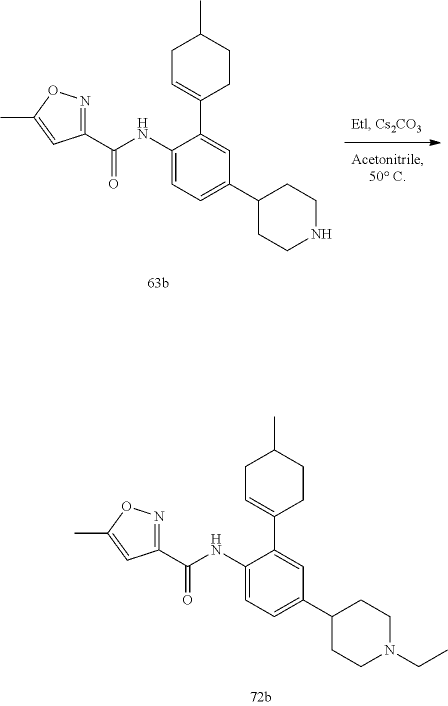 [SMILES: Cc1cc(C(=O)Nc2ccc(C3CCNCC3)cc2C2=CCC(C)CC2)no1 ; CCN1CCC(c2ccc(NC(=O)c3cc(C)on3)c(C3=CCC(C)CC3)c2)CC1]

The synthesis was performed in the same manner as in Example 71 b, and Prep TLC (developing solvent: MC:MeOH=10:1) was performed to obtain Compound 72b (6.9 mg, 0.017 mmol, 51%).

1H NMR (400 MHz, DMSO-d6) δ 9.63 (s, 1H), 7.69 (d, J=8.3 Hz, 1H), 7.18 (d, J=9.6 Hz, 1H), 7.10 (s, 1H), 6.64 (s, 1H), 5.68 (s, 1H), 2.93 (s, 2H), 2.73 (s, 1H), 2.52 (s, 3H), 2.22 (d, J=16.3 Hz, 4H), 1.97 (dd, J=22.8, 9.1 Hz, 4H), 1.74 (d, J=13.1 Hz, 4H), 1.43 (s, 2H), 1.32 (s, 1H), 1.20 (d, J=2.7 Hz, 3H), 1.00 (d, J=6.1 Hz, 3H).

HRMS(ESI+) calculated for C25H34N3O2 [M+H]+: 408.2646, found 408.1492.

### N-(5-(1-ethylpiperidin-4-yl)-4′,4′-dimethyl-2′,3′,4′,5′-tetrahydro-[1,1′-biphenyl]-2-yl)-5-methylisoxazole-3-carboxamide. (73b)

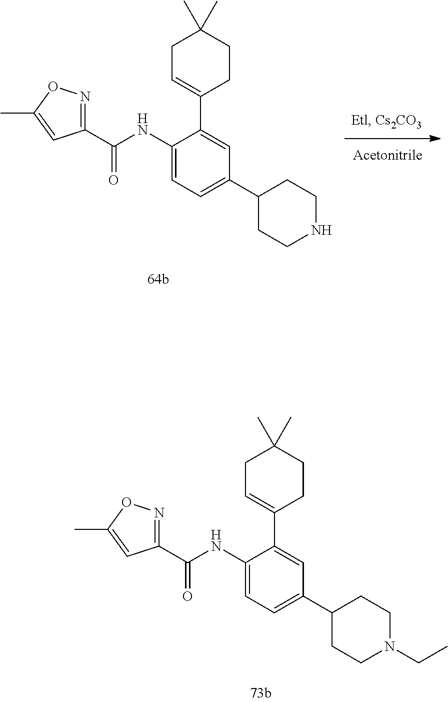 [SMILES: Cc1cc(C(=O)Nc2ccc(C3CCNCC3)cc2C2=CCC(C)(C)CC2)no1 ; CCN1CCC(c2ccc(NC(=O)c3cc(C)on3)c(C3=CCC(C)(C)CC3)c2)CC1]

Compound 64b (15 mg, 0.038 mmol) and Cessium carbonate (7.9 mg, 0.057 mmol) were dissolved in Acetonitrile (0.95 mL), Iodoethane (3.05 μL, 0.038 mol) was added dropwise, and the resulting mixture was stirred for 12-24 hours. After confirming the completion of the reaction, the reaction mixture was filtered under reduced pressure with MC:MeOH=10:1 and concentrated. Separation and purification by column chromatography yielded Compound 73b (11.9 mg, 0.029 mmol, 76%).

1H NMR (400 MHz, DMSO-d6) δ 9.64 (s, 1H), 7.67 (dd, J=11.8, 8.3 Hz, 1H), 7.16 (d, J=8.4 Hz, 1H), 7.09 (s, 1H), 6.60 (d, J=0.9 Hz, 1H), 5.61 (s, 1H), 3.53 (s, 2H), 3.20-3.06 (m, 2H), 2.99 (s, 2H), 2.81 (s, 1H), 2.49-2.48 (m, 3H), 2.24 (s, 2H), 2.00 (d, J=8.4 Hz, 2H), 1.90 (s, 2H), 1.82 (d, J=11.4 Hz, 2H), 1.44 (t, J=6.3 Hz, 2H), 1.23 (s, 3H), 0.94 (s, 6H).

HRMS (ESI+) calculated for C26H36N3O2 [M+H]+: 422.2802, found 422.5186.

### N-(2-cyclohexyl-4-(1-ethylpiperidin-4-yl)phenyl)-5-methylisoxazole-3-carboxamide. (74b)

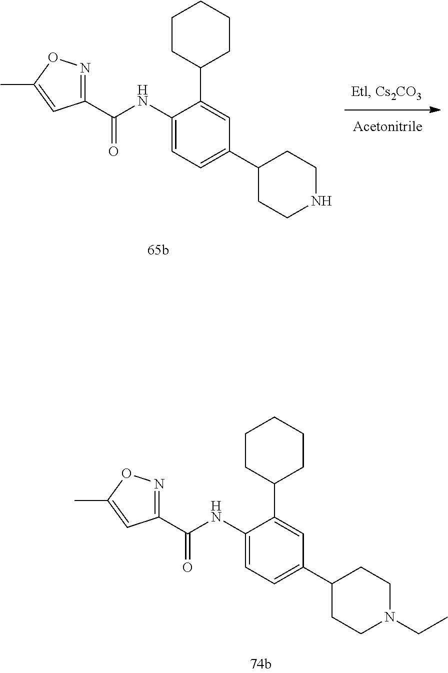 [SMILES: Cc1cc(C(=O)Nc2ccc(C3CCNCC3)cc2C2CCCCC2)no1 ; CCN1CCC(c2ccc(NC(=O)c3cc(C)on3)c(C3CCCCC3)c2)CC1]

The obtained compound was synthesized in the same manner as in Example 73b, and separated and purified by column chromatography to obtain Compound 74b (6 mg, 0.015 mmol, 57%).

1H NMR (400 MHz, MeOD) δ 7.35 (d, J=8.1 Hz, 1H), 7.28 (d, J=1.9 Hz, 1H), 7.16 (dd, J=8.2, 2.0 Hz, 1H), 6.54 (d, J=0.9 Hz, 1H), 3.61 (d, J=12.4 Hz, 2H), 3.17 (q, J=7.3 Hz, 2H), 3.02 (t, J=11.6 Hz, 2H), 2.92 (ddd, J=15.8, 8.0, 3.7 Hz, 1H), 2.78 (t, J=11.2 Hz, 1H), 2.53 (d, J=0.7 Hz, 3H), 2.11 (d, J=14.0 Hz, 2H), 1.98 (dt, J=23.4, 7.2 Hz, 2H), 1.80 (dd, J=30.6, 11.1 Hz, 5H), 1.37 (s, 8H).

HRMS (ESI+) calculated for C24H34N3O2 [M+H]+: 396.2646, found 396.2286.

### N-(2-Cyclopentyl-4-(1-ethylpiperidin-4-yl)phenyl)-5-methylisoxazole-3-carboxamide (75b)

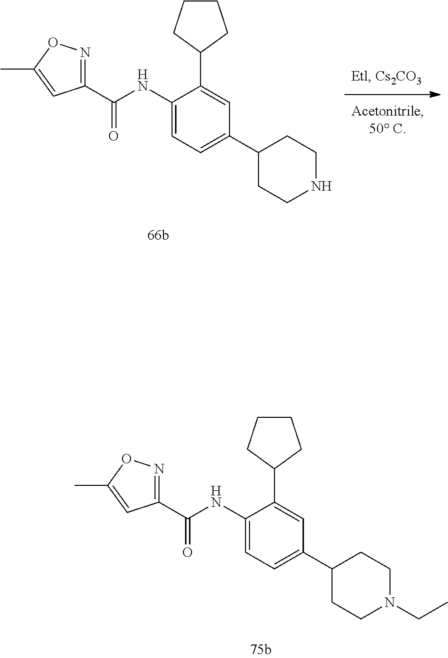 [SMILES: Cc1cc(C(=O)Nc2ccc(C3CCNCC3)cc2C2CCCC2)no1 ; CCN1CCC(c2ccc(NC(=O)c3cc(C)on3)c(C3CCCC3)c2)CC1]

The synthesis was performed in the same manner as in Example 70b, and Prep TLC (developing solvent: MC:MeOH=10:1) was performed to obtain Compound 75b (5.6 mg, 0.015 mmol, 56%).

1H NMR (400 MHz, DMSO-d6) δ 10.07 (s, 1H), 7.20 (d, J=1.8 Hz, 1H), 7.17 (d, J=8.1 Hz, 1H), 7.08 (dd, J=8.1, 1.9 Hz, 1H), 6.63 (d, ^{J=0.9} Hz, 1H), 3.16 (d, J=7.3 Hz, 1H), 3.07 (d, J=10.1 Hz, 2H), 2.60-2.52 (m, 1H), 2.50 (s, 3H), 2.21-2.10 (m, 2H), 1.93 (dd, J=8.0, 7.5 Hz, 2H), 1.85-1.63 (m, 7H), 1.61-1.43 (m, 5H), 1.06 (t, J=7.2 Hz, 3H).

HRMS(ESI+) calculated for C23H32N3O2 [M+H]+: 382.2489, found 382.9754.

### 5-methyl-N-(5-(1-(3-methylbut-2-en-1-yl)piperidin-4-yl)-2′,3′,4′,5′-tetrahydro-[1,1′-biphenyl]-2-yl)isoxazole-3-carboxamide. (76b)

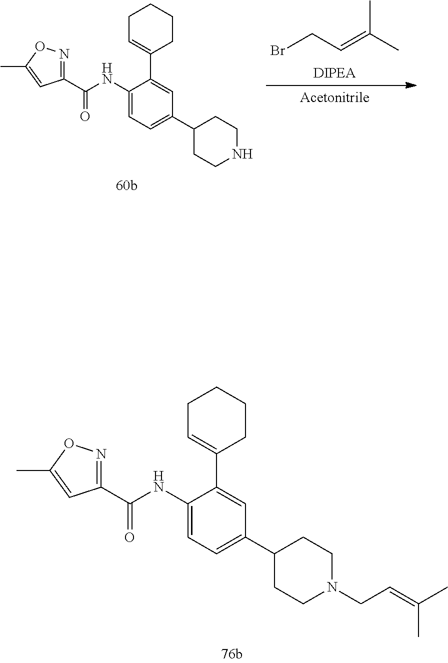

Compound 60b (14 mg, 0.038 mmol) obtained above was dissolved in Acetonitrile (0.38 mL), DIPEA (19.9 μL, 0.114 mmol) was added thereto, and compound 1-bromo-3-methylbut-2-ene (6.59 μL, 0.057 mmol) was added dropwise, and the resulting mixture was stirred for 1 hour. After the completion of the reaction was confirmed, the reaction product was extracted with Ethyl acetate and washed with 1M NaOH and brine. Dried over Anhydrous Na2SO4 and concentrated. Separation and purification by column chromatography (MC:MeOH=10:1) yielded Compound 76b (6 mg, 0.0138 mmol, 36%).

1H NMR (400 MHz, CDCl3) δ 9.00 (s, 1H), 8.29 (d, J=8.4 Hz, 1H), 7.14 (dd, J=8.4, 2.1 Hz, 1H), 7.02 (d, J=2.0 Hz, 1H), 6.50 (d, J=0.8 Hz, 1H), 5.80 (s, 1H), 5.34 (s, 1H), 3.15 (s, 3H), 2.49 (d, J=0.6 Hz, 3H), 2.29-2.20 (m, 4H), 2.03 (d, J=6.8 Hz, 1H), 1.88 (s, 2H), 1.82 (dd, J=10.4, 5.0 Hz, 2H), 1.77 (s, 3H), 1.76-1.70 (m, 3H), 1.68 (s, 3H), 1.63 (s, 4H).

HRMS (ESI+) calculated for C27H36N3O2 [M+H]+: 434.2802, found 434.0787.

### N-(5-(1-(cyclopropylmethyl)piperidin-4-yl)-4′-methyl-2,3,4,5′-tetrahydro-[1,1′-biphenyl]-2-yl)-5-methylisoxazole-3-carboxamide. (77b)

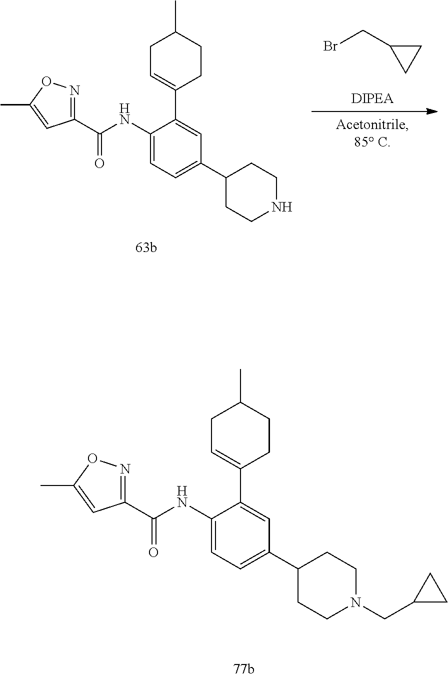 [SMILES: Cc1cc(C(=O)Nc2ccc(C3CCNCC3)cc2C2=CCC(C)CC2)no1 ; BrCC1CC1 ; Cc1cc(C(=O)Nc2ccc(C3CCN(CC4CC4)CC3)cc2C2=CCC(C)CC2)no1]

Compound 63b (10.9 mg, 0.0287 mmol) obtained above was dissolved in Acetonitrile (0.72 mL), and DIPEA (50 μL) was obtained. 0.287 mmol) of bromomethyl cyclopropane (2.75 μL, 0.0.287 mmol) was added dropwise thereto, and the result was stirred at 85° C. for 3 hours. After the completion of the reaction was confirmed, the reaction mixture was transferred to room temperature, extracted with Ethyl acetate, washed with water and brine, dried over Anhydrous Na2SO4, and concentrated. Separation and purification by column chromatography yielded Compound 77b (6 mg, 0.0138 mmol, 48%).

1H NMR (400 MHz, DMSO-d6) δ 9.57 (s, 1H), 7.66 (dd, J=11.5, 8.3 Hz, 1H), 7.16 (d, J=6.6 Hz, 1H), 7.08 (s, 1H), 6.63 (t, J=5.5 Hz, 1H), 5.65 (s, 1H), 3.09 (s, 2H), 2.53-2.51 (m, 1H), 2.49-2.49 (m, 3H), 2.48-2.46 (m, 2H), 2.20 (d, J=16.0 Hz, 3H), 2.01 (s, 2H), 1.72 (d, J=13.3 Hz, 4H), 1.69-1.60 (m, 2H), 1.33 (s, 2H), 0.98 (d, J=6.0 Hz, 3H), 0.86 (d, J=2.7 Hz, 1H), 0.48 (s, 2H), 0.10 (s, 2H).

HRMS (ESI+) calculated for C27H36N3O2 [M+H]+: 434.2802, found 434.6109.

### N-(5-(1-(cyclopropylmethyl)piperidin-4-yl)-4′,4′-dimethyl-2′,3′,4′,5′-tetrahydro-[1,1′-biphenyl]-2-yl)-5-methylisoxazole-3-carboxamide. (78b)

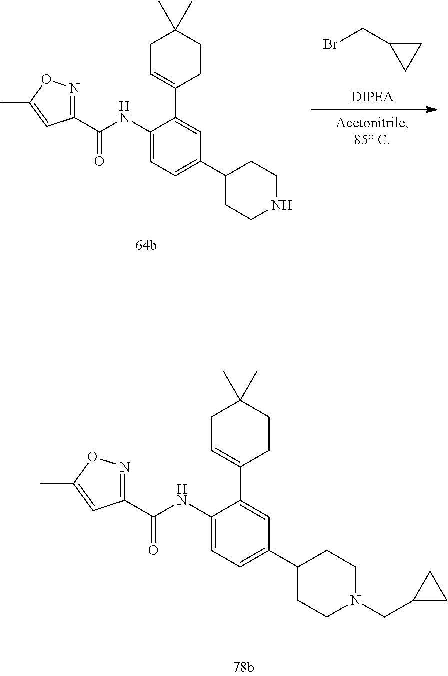 [SMILES: Cc1cc(C(=O)Nc2ccc(C3CCNCC3)cc2C2=CCC(C)(C)CC2)no1 ; BrCC1CC1 ; Cc1cc(C(=O)Nc2ccc(C3CCN(CC4CC4)CC3)cc2C2=CCC(C)(C)CC2)no1]

The reaction mixture was synthesized in the same manner as above, and separated and purified through column chromatography to obtain Compound 78b (8.6 mg, 0.018 mmol, 50%).

1H NMR (400 MHz, CDCl3) δ 9.01 (s, 1H), 8.31 (d, J=8.4 Hz, 1H), 7.16 (dd, J=8.4, 2.1 Hz, 1H), 7.03 (d, J=2.1 Hz, 1H), 6.50 (d, J=0.9 Hz, 1H), 5.75-5.71 (m, 1H), 3.24 (s, 2H), 2.49 (d, J=0.8 Hz, 4H), 2.34 (s, 1H), 2.26 (dd, J=8.2, 6.2 Hz, 2H), 2.11 (s, 1H), 2.04 (d, J=3.6 Hz, 2H), 1.86 (s, 3H), 1.63 (s, 3H), 1.55 (t, J=6.3 Hz, 2H), 1.04 (s, 6H), 0.94 (s, 1H), 0.55 (d, J=7.5 Hz, 2H), 0.15 (d, J=3.9 Hz, 2H).

HRMS (ESI+) calculated for C28H38N3O2 [M+H]+: 448.2959, found 448.9519.

### 1,1-dimethyl-4-(6-(5-methylisoxazole-3-carboxamido)-2′,3′,4′,5′-tetrahydro-[1,1′-biphenyl]-3-yl)piperidin-1-ium. (79b)

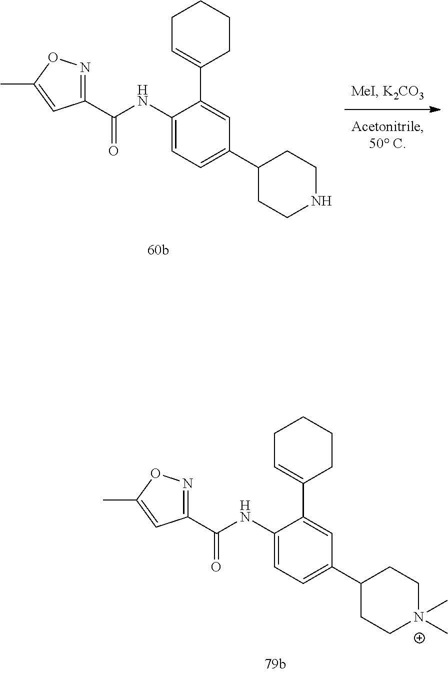

Compound 60b (10 mg, 0.028 mmol) and Potassium carbonate (9.7 mg, 0.07 mmol) were dissolved in Acetonitrile (0.7 mL), and Iodomethane (3.4 μL, 0.056 mmol) was added dropwise at 50° C., followed by stirring for 3 hours. After confirming the completion of the reaction, it was moved to room temperature, filtered with Ethyl acetate, and concentrated. Separation and purification by column chromatography yielded Compound 79b (10.2 mg, 0.0285 mmol, 92%).

1H NMR (400 MHz, DMSO-d6) δ 9.62 (s, 1H), 7.70 (d, J=8.3 Hz, 1H), 7.27 (d, J=8.3 Hz, 1H), 7.22 (d, J=2.0 Hz, 1H), 6.63 (s, 1H), 5.70 (s, 1H), 3.52 (d, J=12.3 Hz, 2H), 3.44 (d, J=13.1 Hz, 2H), 3.16 (d, J=4.4 Hz, 6H), 2.78 (s, 1H), 2.51 (s, 3H), 2.23-2.21 (m, 1H), 1.97 (s, 2H), 1.94 (s, 2H), 1.91-1.90 (m, 1H), 1.69 (d, J=5.9 Hz, 2H), 1.62 (d, J=5.3 Hz, 2H), 1.47-1.43 (m, 2H).

HRMS (ESI+) calculated for C24H32N3O2 [M+H]+: 394.2489, found 394.4279.

### 1,1-dimethyl-4-(6-(5-methylisoxazole-3-carboxamido)-2′,3′,4′,5′-tetrahydro-[1,1′-biphenyl]-3-yl)azepan-1-ium. (79c)

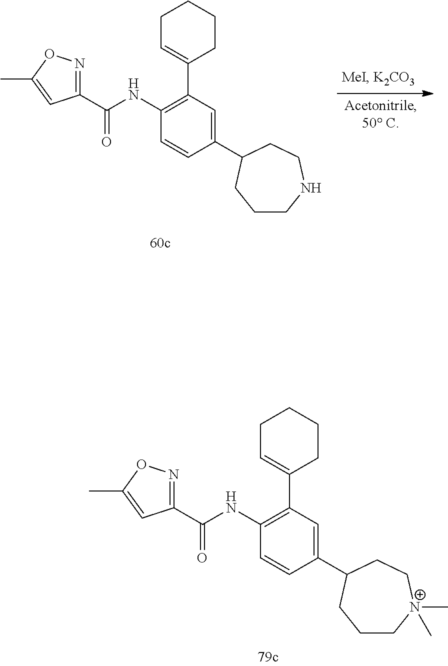

The reaction mixture was synthesized in the same manner as above, and separated and purified through column chromatography to obtain 13 mg (0.318 mmol) of Compound 79c (Yield: 81%).

1H NMR (400 MHz, DMSO-d6) δ 9.61 (s, 1H), 7.68 (d, J=8.2 Hz, 1H), 7.23-7.15 (m, 2H), 6.65 (s, 1H), 5.71 (s, 1H), 3.61-3.51 (m, 4H), 3.13 (d, J=10.2 Hz, 6H), 2.80 (s, 1H), 2.51-2.51 (m, 3H), 2.35 (s, 1H), 2.23 (s, 2H), 2.13 (s, 2H), 2.00-1.85 (m, 4H), 1.71 (d, J=4.1 Hz, 2H), 1.63 (s, 3H).

HRMS(ESI+) calculated for C25H34N3O2 [M+H]+: 408.2646, found 408.7575.

### 4-(4′,4′-dimethyl-6-(5-methylisoxazole-3-carboxamido)-2′,3′,4′,5′-tetrahydro-[1,1′-biphenyl]-3-yl)-1,1-dimethylazepan-1-ium. (80c)

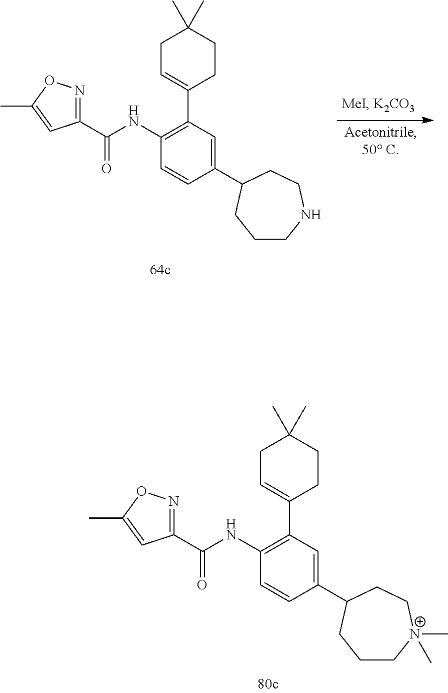

The reaction mixture was synthesized in the same manner as above, and separated and purified through column chromatography to obtain 11.3 mg (0.0258 mmol, 76%) of Compound 80c.

1H NMR (400 MHz, DMSO-d6) δ 9.61 (s, 1H), 7.64 (dd, J=11.5, 8.1 Hz, 1H), 7.21-7.17 (m, 1H), 7.15 (d, J=1.8 Hz, 1H), 6.60 (d, J=0.8 Hz, 1H), 5.61 (s, 1H), 3.59-3.45 (m, 4H), 3.11 (d, J=10.1 Hz, 6H), 2.77 (d, J=12.1 Hz, 1H), 2.49-2.48 (m, 3H), 2.32 (d, J=8.5 Hz, 1H), 2.25 (s, 2H), 1.98-1.82 (m, 6H), 1.61 (dd, J=23.3, 11.9 Hz, 1H), 1.44 (t, J=6.3 Hz, 2H), 0.95 (s, 6H).

HRMS(ESI+) calculated for C27H38N3O2 [M+H]+: 436.2959, found 436.8157.

### 4-(3-cyclohexyl-4-(5-methylisoxazole-3-carboxamido)phenyl)-1,1-dimethylpiperidin-1-ium. (81b)

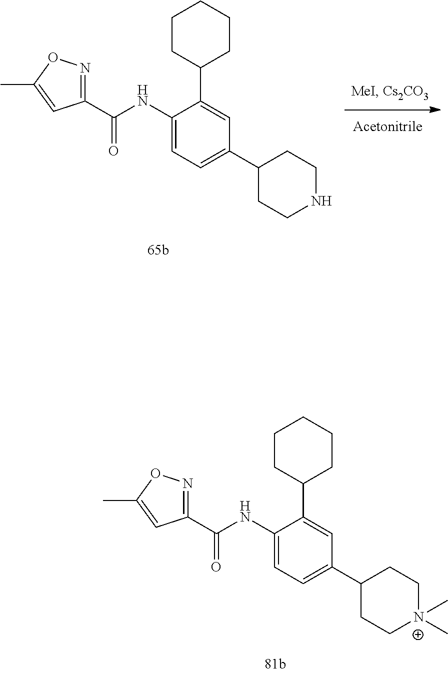

Compound 65b (10 mg, 0.027 mmol) and Cessium carbonate (13 mg, 0.405 mmol) were dissolved in Acetonitrile (0.68 mL), Iodomethane (1.68 μL, 0.027 mmol) was added dropwise, and the resulting mixture was stirred for 7 hours. After confirming the completion of the reaction, the reaction mixture was filtered with MC:MeOH=10:1 and concentrated. Separation and purification by column chromatography yielded Compound 81 b (5 mg, 0.0126 mmol, 46.7%).

1H NMR (400 MHz, MeOD) δ 7.37 (d, J=8.2 Hz, 1H), 7.34 (d, J=2.0 Hz, 1H), 7.25 (dd, J=8.2, 2.1 Hz, 1H), 6.54 (d, J=0.9 Hz, 1H), 3.65-3.52 (m, 4H), 3.25 (s, 6H), 2.95 (ddd, J=12.4, 8.1, 3.8 Hz, 1H), 2.82-2.74 (m, 1H), 2.53 (d, J=0.8 Hz, 3H), 2.23 (ddd, J=27.9, 12.7, 4.4 Hz, 2H), 2.04 (d, J=16.2 Hz, 2H), 1.80 (dd, J=30.6, 10.5 Hz, 5H), 1.54-1.34 (m, 5H).

HRMS (ESI+) calculated for C24H34N3O2 [M+H]+: 396.2646, found 396.5888

### 1,1-diethyl-4-(6-(5-methylisoxazole-3-carboxamido)-2′,3′,4′,5′-tetrahydro-[1,1′-biphenyl]-3-yl)piperidin-1-ium. (82b)

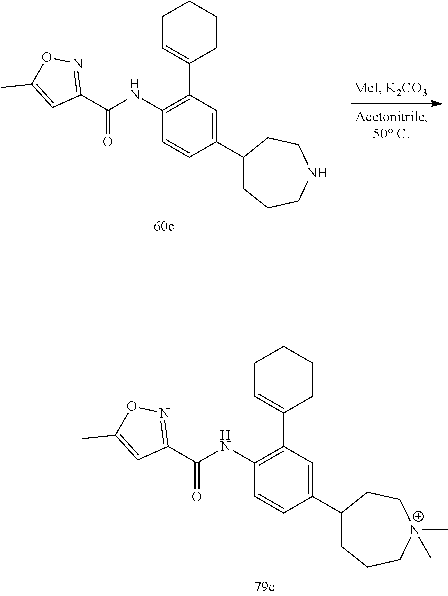

In the present invention, Compound 60b (10 mg, 0.028 mmol) obtained above and Potassium carbonate (9.7 mg, 0.07 mmol) were added and dissolved in Acetonitrile (0.7 mL), and then Iodoethane (4.5 μL, 0.56 mmol) was dropped at 50° C. and stirred for 24-48 hours.

After confirming the completion of the reaction, it was moved to room temperature, filtered with Ethyl acetate, and concentrated. The invention relates to separation and purification using column chromatography to afford compound 82b (10 mg, 0.0237 mmol, 84%) 1H NMR (400 MHz, DMSO-d6) δ 9.62 (s, 1H), 7.71-7.67 (m, 1H), 7.29-7.25 (m, 1H), 7.23 (d, J=1.9 Hz, 1H), 6.63 (s, 1H), 5.70 (s, 1H), 3.54 (dd, J=18.8, 11.5 Hz, 4H), 3.33 (d, J=9.5 Hz, 4H), 2.82 (d, J=12.9 Hz, 1H), 2.50 (s, 3H), 2.23-2.21 (m, 1H), 2.10 (s, 2H), 1.97 (s, 1H), 1.94 (s, 1H), 1.87 (s, 1H), 1.70 (d, J=5.7 Hz, 2H), 1.62 (d, J=5.6 Hz, 2H), 1.45 (s, 2H), 1.18 (td, J=7.1, 2.9 Hz, 6H).

HRMS (ESI+) calculated for C26H36N3O2 [M+H]+: 422.2802, found 422.3385.

### N-(5-(1-acetylpiperidin-4-yl)-2′,3′,4′,5′-tetrahydro-[1,1′-biphenyl]-2-yl)-5-methylisoxazole-3-carboxamide. (83b)

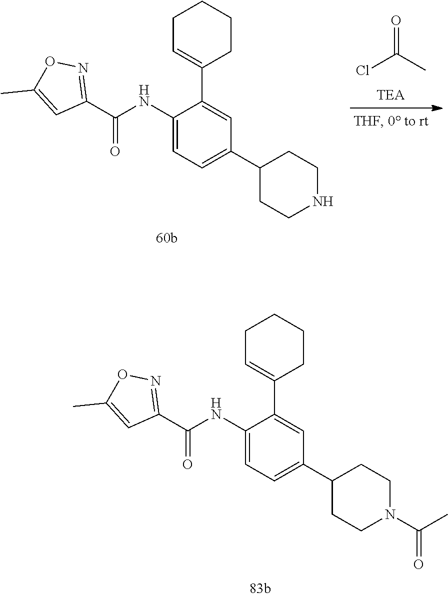

Compound 60b (10 mg, 0.02 mmol) obtained above was dissolved in THE (0.2 mL), Triethylamine (8.16 L, 0.06 mmol) was added thereto, Acetyl chloride (1.71 μL, 0.024 mmol) was added dropwise thereto at 0° C., and the resulting mixture was transferred to room temperature, and then stirred for 1 hour. Extracted with Ethyl acetate and washed with water and brine. After drying with Anhydrous Na2SO4, concentration afforded Compound 83b (8 mg, 0.0196 mmol, 98%).

1H NMR (400 MHz, DMSO-d6) δ 9.57 (s, 1H), 7.66 (d, J=8.2 Hz, 1H), 7.18-7.13 (m, 1H), 7.08 (d, J=2.0 Hz, 1H), 6.63 (d, J=0.8 Hz, 1H), 5.68 (s, 1H), 4.52 (d, J=13.1 Hz, 1H), 3.91 (d, J=13.6 Hz, 1H), 3.11 (t, J=11.9 Hz, 1H), 2.75 (ddd, J=12.0, 8.7, 3.6 Hz, 1H), 2.56 (dd, J=14.8, 12.6 Hz, 1H), 2.48 (s, 3H), 2.20 (s, 2H), 2.09 (s, 2H), 2.03 (d, J=2.3 Hz, 3H), 1.76 (d, J=12.9 Hz, 2H), 1.69-1.64 (m, 2H), 1.64-1.57 (m, 3H), 1.43 (dd, J=12.6, 4.2 Hz, 1H).

HRMS (ESI+) calculated for C24H30N3O3 [M+H]+: 408.2282, found 408.5093.

### N-(5-(1-(dimethylglycyl)piperidin-4-yl)-2′,3′,4′,5′-tetrahydro-[1,1′-biphenyl]-2-yl)-5-methylisoxazole-3-carboxamide. (84b)

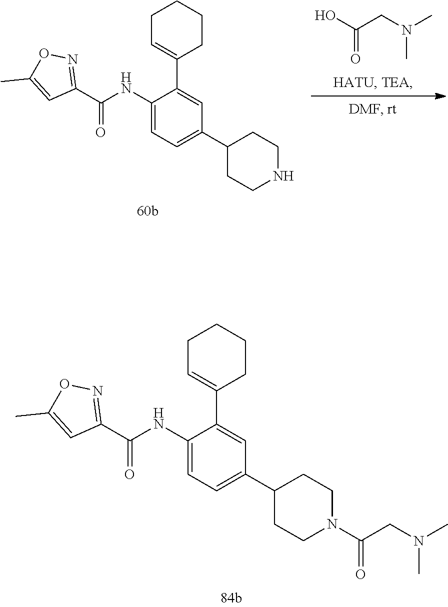

Dimethylglycine (4.17 mg, 0.0405 mmol) and HATU (18.5 mg, 0.486 mmol) were dissolved in DMF (0.27 mL), Triethylamine (9.1 μL, 0.0675 mmol) was added thereto, and the resulting mixture was stirred at room temperature for 1 hour. Compound 60b (10 mg, 0.027 mmol) obtained above was added and stirred. After confirming the completion of the reaction, the reaction mixture was extracted with Ethyl acetate and washed with water and brine. After drying with Anhydrous Na2SO4, the mixture was concentrated. Separation and purification by column chromatography (MC:MeOH=40:1) yielded Compound 84b (10.5 mg, 0.0233 mmol, 86%).

1H NMR (400 MHz, DMSO-d6) δ 9.59 (s, 1H), 7.66 (d, J=8.3 Hz, 1H), 7.15 (dd, J=8.3, 2.0 Hz, 1H), 7.07 (d, J=2.0 Hz, 1H), 6.62 (d, J=0.8 Hz, 1H), 5.68 (s, 1H), 4.51 (d, J=12.9 Hz, 1H), 3.87 (dd, J=19.0, 11.1 Hz, 3H), 3.12 (t, J=12.1 Hz, 1H), 2.81 (t, J=12.1 Hz, 1H), 2.70 (dd, J=19.4, 7.2 Hz, 1H), 2.61 (s, 6H), 2.49 (s, 3H), 2.20 (s, 2H), 2.09 (d, J=3.7 Hz, 2H), 1.83 (d, J=11.8 Hz, 2H), 1.69-1.59 (m, 5H), 1.51-1.42 (m, 1H).

HRMS (ESI+) calculated for C26H35N4O3 [M+H]+: 451.2704, found 451.5448.

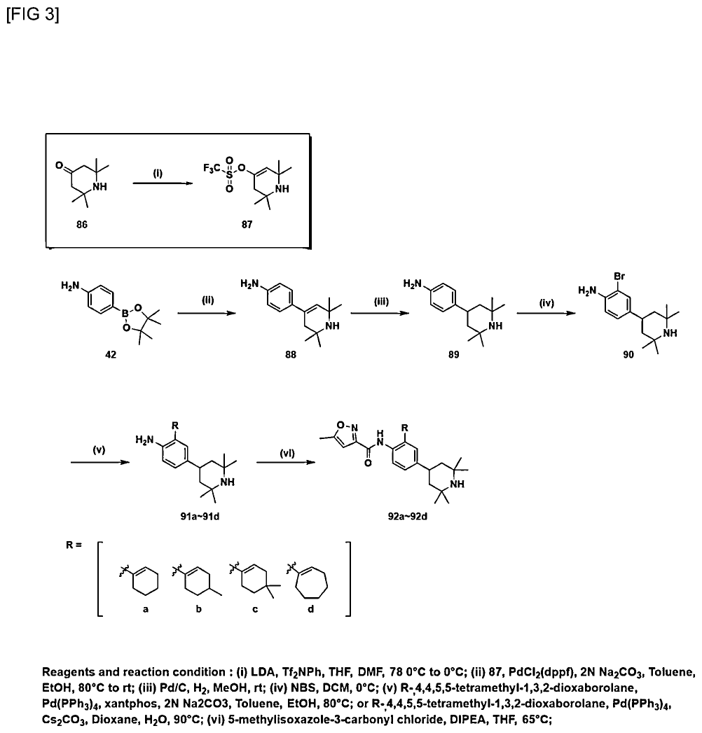

### 4. Synthesis of Tetramethylpiperidine Derivatives (Scheme 3, FIG. 3)

TABLE 2

| Structure | NO. | R | IC₅₀ (μm) |
|---|---|---|---|
| 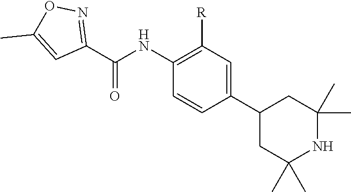 | 92a | 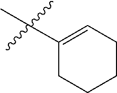 [SMILES: *C1=CCCCC1] | ++ |
| | 92b | 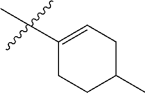 [SMILES: *C1=CCC(C)CC1] | +++ |
| | 92c | 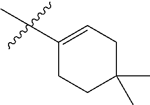 [SMILES: *C1=CCC(C)(C)CC1] | +++ |
| | 92d | 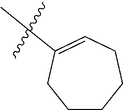 [SMILES: *C1=CCCCCC1] | ++ |

### 2,2,6,6-tetramethyl-1,2,3,6-tetrahydropyridin-4-yl trifluoromethanesulfonate. (87)

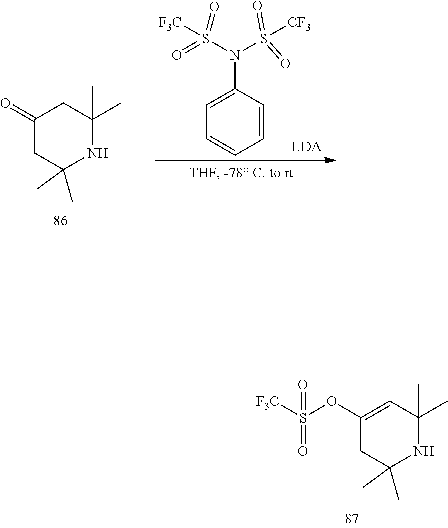 [SMILES: CC1(C)CC(=O)CC(C)(C)N1 ; O=S(=O)(N(c1ccccc1)S(=O)(=O)C(F)(F)F)C(F)(F)F ; CC1(C)C=C(OS(=O)(=O)C(F)(F)F)CC(C)(C)N1]

In the present invention, 2M LDA (4.83 mL) was added at −78° C., 2,2,6,6-tetramethylpiperidin-4-one (1 g, 6.44 mmol) was dissolved in THE (10 mL), and then the result was dropped and stirred for 1 hour. N-Phenylbis (trifluoromethanesulfonimide) (2.530 g, 7.084 mmol) was dissolved in THE (10 mL), and the resulting solution was slowly added thereto, followed by stirring at 12h-24h. After being transferred to room temperature, the reaction mixture was extracted with Ethyl acetate, washed with water, NH4Cl, brine, dried over Anhydrous Na2SO4, and concentrated. This document is separated and purified by column chromatography to obtain compound 87 (1.1146 mg, 60%) 1H NMR (400 MHz, CDCl3) δ 5.69 (t, J=1.3 Hz, 1H), 2.45 (s, 1H), 2.18 (d, J=1.3 Hz, 2H), 1.27 (s, 6H), 1.22 (s, 6H).

HRMS(ESI+) calculated for C10H17F3NO3S [M+H]+: 288.0876, found 288.8850.

### 4-(2,2,6,6-tetramethyl-1,2,3,6-tetrahydropyridin-4-yl)aniline. (88)

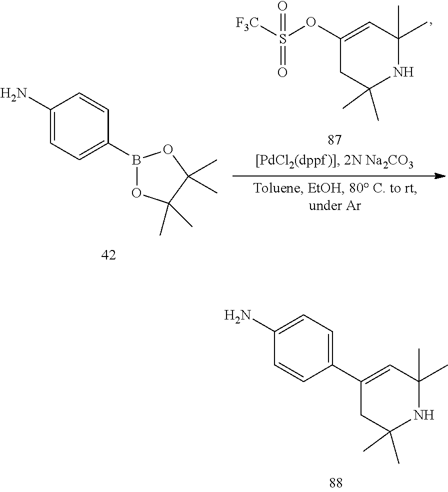 [SMILES: CC1(C)OB(c2ccc(N)cc2)OC1(C)C ; CC1(C)C=C(OS(=O)(=O)C(F)(F)F)CC(C)(C)N1 ; CC1(C)C=C(c2ccc(N)cc2)CC(C)(C)N1]

In the present invention, Compound 87 (920 mg, 3.2 mmol) obtained above and [PdCl2(dppf)] (109 mg, 0.32 mmol) were added and dissolved using Toluene (23.84 mL) and Ethanol (11.86 mL), 2N Na₂CO3 (11.75 mL, 23.5 mmol) was added thereto, and then 4-(4, 4, 5, 5-tetramethyl-1, 3, 2-dioxaborolan-2-yl)aniline (585 mg, 2.67 mmol) was added thereto at 80° C., followed by stirring for 2 hours. After confirming the completion of the reaction, the reaction mixture was transferred to room temperature and cooled, and then extracted with Ethyl acetate, and washed with NaHCO₃, water, and brine. It was dried over Anhydrous Na2SO4, concentrated, and separated and purified using column chromatography (MC:MeOH=20:1) to obtain Compound 88 (492 mg, 80%)

1H NMR (400 MHz, DMSO-d6) δ 7.07 (d, J=8.5 Hz, 2H), 6.54-6.49 (m, 2H), 5.77 (s, 1H), 5.03 (d, J=8.2 Hz, 2H), 2.10 (s, 2H), 1.14 (d, J=20.3 Hz, 13H).

HRMS(ESI+) calculated for C15H23N2 [M+H]+: 231.1856, found 231.4503.

### 4-(2,2,6,6-tetramethylpiperidin-4-yl)aniline. (89)

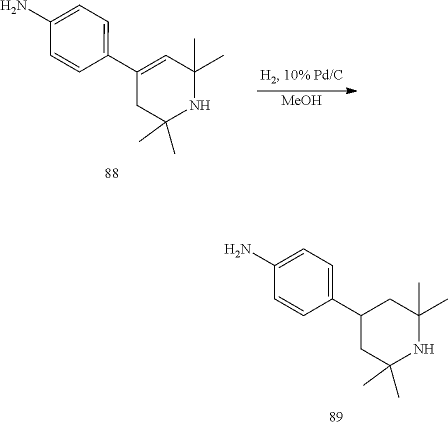 [SMILES: "MeOH" ; CC1(C)C=C(c2ccc(N)cc2)CC(C)(C)N1 ; CC1(C)CC(c2ccc(N)cc2)CC(C)(C)N1]

Compound 88 (994 mg, 4.32 mmol) was dissolved in Methanol (21.6 mL), Pd/C (99.4 mg) was added thereto, and the mixture was substituted with hydrogen gas and stirred for 1-4 hours. After the completion of the reaction was confirmed, Pd/C was removed using cellite and concentrated to give Compound 89 (932 mg, 92%).

1H NMR (400 MHz, DMSO-d6) δ 6.87 (d, J=8.4 Hz, 2H), 6.51-6.47 (m, 2H), 4.81 (s, 2H), 2.82 (t, J=12.6 Hz, 1H), 1.55 (dd, J=12.8, 2.7 Hz, 2H), 1.16 (d, J=51.6 Hz, 15H).

HRMS(ESI+) calculated for C15H25N2 [M+H]+: 233.2012, found 233.8475.

### 2-bromo-4-(2,2,6,6-tetramethylpiperidin-4-yl)aniline. (90)

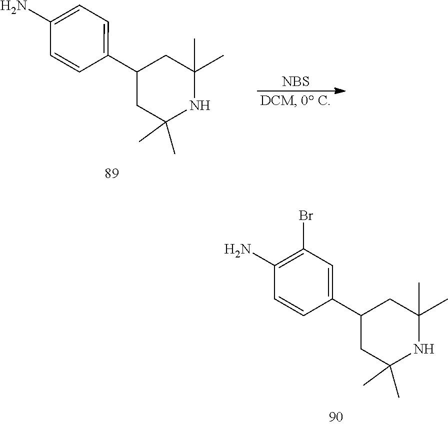

Compound 89 (767 mg. 3.3 mmol) was dissolved in DCM (66 mL), and N-Bromosuccinimde (587 mg, 3.3 mmol) was divided into four times at 0° C., and slowly added thereto at intervals of 30 minutes to perform reaction. After the completion of the reaction was confirmed, the reaction product was extracted with Dichloromethane and washed with NaHCO₃ and water. It was dried over Anhydrous Na2SO4, concentrated, separated and purified using column chromatography to afford Compound 90 (235 mg, 22.7%).

1H NMR (400 MHz, DMSO-d6) δ 7.17 (d, J=2.0 Hz, 1H), 6.93 (dd, J=8.3, 2.0 Hz, 1H), 6.72 (d, J=8.2 Hz, 1H), 5.04 (d, J=11.3 Hz, 2H), 2.83 (t, J=12.7 Hz, 1H), 1.52 (dd, J=12.7, 3.1 Hz, 2H), 1.19-1.09 (m, 9H), 1.03 (s, 6H).

HRMS(ESI+) calculated for C15H24BrN2 [M+H]+: 311.1117, found 311.3750.

### 5-(2,2,6,6-tetramethylpiperidin-4-yl)-2,3,4,5′-tetrahydro-[1,1′-biphenyl]-2-amine. (91a)

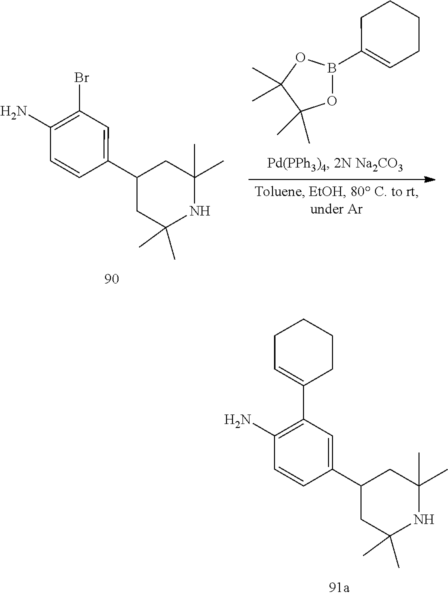 [SMILES: CC1(C)CC(c2ccc(N)c(Br)c2)CC(C)(C)N1 ; CC1(C)OB(C2=CCCCC2)OC1(C)C ; CC1(C)CC(c2ccc(N)c(C3=CCCCC3)c2)CC(C)(C)N1]

After dissolving Compound 90 (300 mg, 0.96 mmol), Pd(PPh3)4 (55 mg, 0.048 mmol), and 2N Na2CO3 (2.1 mL) in Toluene (4.28 mL) and Ethanol (2.14 mL), 2-(1-Cyclohexyl)-4, 4, 5, 5-tetramethyl-1, 3, 2-dioxaborolane (104 μL, 0.48 mmol) was added dropwise and stirred at 80° C. After confirming the completion of the reaction, it was transferred to room temperature and cooled, extracted with Ethyl acetate, and washed with water and brine. It was dried over Anhydrous Na2SO4, concentrated, and separated and purified using column chromatography to obtain 69 mg (46%) of Compound 91a.

1H NMR (400 MHz, DMSO-d6) δ 6.77 (dd, J=8.2, 2.1 Hz, 1H), 6.68 (d, J=2.1 Hz, 1H), 6.55 (d, J=8.1 Hz, 1H), 5.59 (s, 1H), 4.42 (d, J=11.3 Hz, 2H), 2.79 (s, 1H), 2.16-2.10 (m, 4H), 1.70 (d, J=3.7 Hz, 2H), 1.63 (d, J=3.8 Hz, 2H), 1.52 (d, J=12.0 Hz, 2H), 1.17 (s, 6H), 1.15 (s, 3H), 1.03 (s, 6H).

HRMS(ESI+) calculated for C₂₁H₃₃N₂ [M+H]+: 313.2638, found 313.5371.

### 4′-methyl-5-(2,2,6,6-tetramethylpiperidin-4-yl)-2′,3′,4′,5′-tetrahydro-[1,1′-biphenyl]-2-amine. (91b)

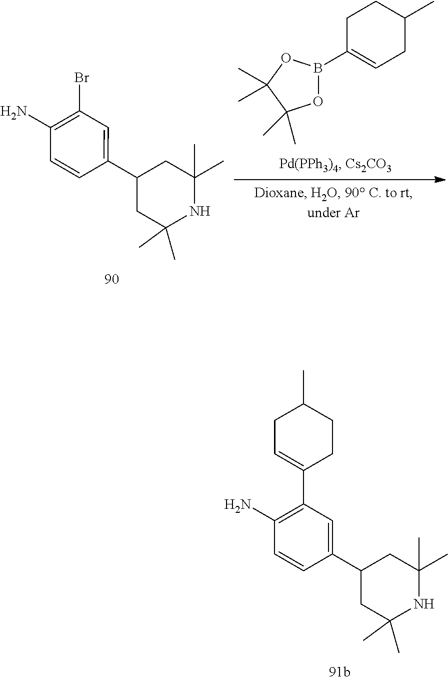

Compound 90 (40 mg, 0.128 mmol) obtained above, Pd(PPh3)4 (15 mg, 0.0128 mmol), and cesium carbonate (133 mg, 0.41 mmol) were dissolved in Dioxane (0.43 mL) and H2O (0.21 mL), and then 4, 4, 5, 5-tetramethyl-2-(4-methylcyclohex-1-en-1-yl)-1, 3, 2-dioxaborolane (57 mg, 0.256 mmol) was added dropwise and stirred at 90° C. After confirming the completion of the reaction, it was transferred to room temperature and cooled, extracted with Ethyl acetate, and washed with water and brine. It was dried over Anhydrous Na2SO4, concentrated, separated and purified using column chromatography to afford Compound 91b (27.7 mg, 66%).

1H NMR (400 MHz, DMSO-d6) δ 6.77 (dd, J=8.2, 2.1 Hz, 1H), 6.68 (d, J=2.0 Hz, 1H), 6.55 (d, J=8.1 Hz, 1H), 5.56 (d, J=2.8 Hz, 1H), 4.41 (d, J=11.0 Hz, 2H), 2.80 (t, J=12.6 Hz, 1H), 2.52 (d, J=4.5 Hz, 1H), 2.18 (t, J=17.4 Hz, 3H), 1.75 (d, J=11.6 Hz, 3H), 1.52 (dd, J=12.8, 2.7 Hz, 2H), 1.34 (d, J=6.3 Hz, 2H), 1.16 (d, J=9.6 Hz, 7H), 1.03 (s, 6H), 0.98 (d, J=6.0 Hz, 3H).

HRMS(ESI+) calculated for C₂₂H₃₅N₂ [M+H]+: 327.2795, found 327.7987.

### 4′,4′-dimethyl-5-(2,2,6,6-tetramethylpiperidin-4-yl)-2′,3′,4′,5′-tetrahydro-[1,1′-biphenyl]-2-amine. (91c)

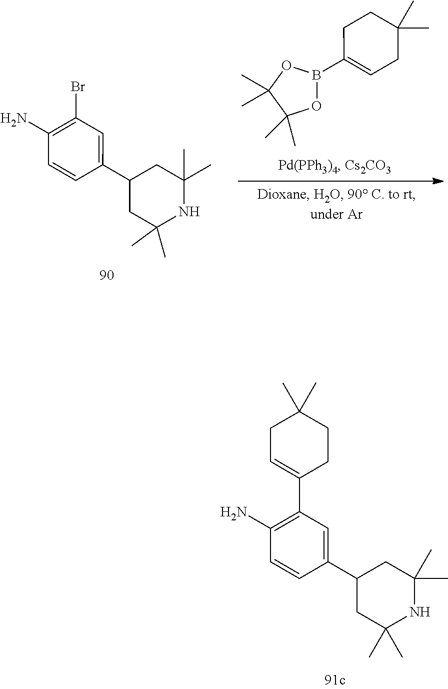

The reaction mixture was synthesized in the same manner as above, and separated and purified through column chromatography to obtain 29 mg (66%) of Compound 91c.

1H NMR (400 MHz, DMSO-d6) δ 6.78 (dd, J=8.2, 2.1 Hz, 1H), 6.69 (d, J=2.1 Hz, 1H), 6.56 (d, J=8.1 Hz, 1H), 5.52 (s, 1H), 4.38 (d, J=11.6 Hz, 2H), 2.80 (t, J=12.6 Hz, 1H), 2.17 (s, 2H), 1.92 (d, J=3.4 Hz, 2H), 1.55-1.44 (m, 4H), 1.25-1.08 (m, 9H), 1.03 (s, 6H), 0.97 (s, 6H).

HRMS(ESI+) calculated for C₂₃H₃₇N₂ [M+H]+: 341.2951, found 341.8820.

### 2-(cyclohept-1-en-1-yl)-4-(2,2,6,6-tetramethylpiperidin-4-yl)aniline. (91d)

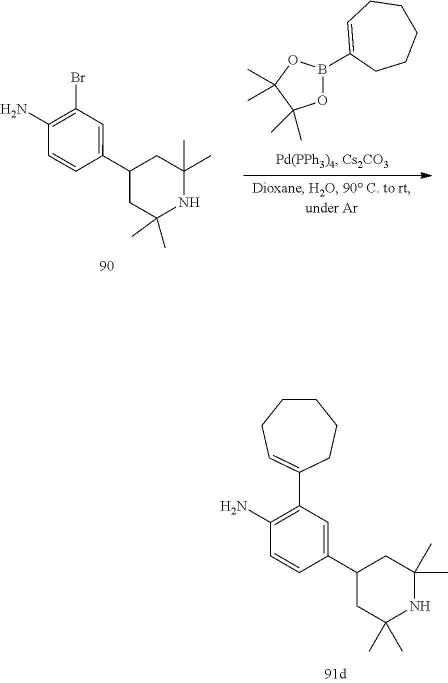 [SMILES: CC1(C)CC(c2ccc(N)c(Br)c2)CC(C)(C)N1 ; CC1(C)OB(C2=CCCCCC2)OC1(C)C ; CC1(C)CC(c2ccc(N)c(C3=CCCCCC3)c2)CC(C)(C)N1]

The reaction mixture was synthesized in the same manner as above, and separated and purified through column chromatography to obtain 35 mg (83%) of Compound 91d.

1H NMR (400 MHz, DMSO-d6) δ 7.17 (d, J=2.0 Hz, 1H), 6.93 (dd, J=8.3, 2.0 Hz, 1H), 6.72 (d, J=8.2 Hz, 1H), 5.76 (t, J=6.5 Hz, 1H), 5.04 (d, J=11.3 Hz, 2H), 2.83 (s, 1H), 2.21 (s, 2H), 1.97 (s, 2H), 1.94 (s, 2H), 1.53 (d, J=3.0 Hz, 2H), 1.50 (d, J=3.1 Hz, 2H), 1.22 (d, J=7.3 Hz, 4H), 1.17 (s, 7H), 1.02 (s, 6H).

HRMS (ESI+) calculated for C₂₂H₃₅N₂ [M+H]+: 327.2795, found 327.3315.

### 5-methyl-N-(5-(2,2,6,6-tetramethylpiperidin-4-yl)-2′,3′,4′,5′-tetrahydro-[1,1′-biphenyl]-2-yl)isoxazole-3-carboxamide. (92a)

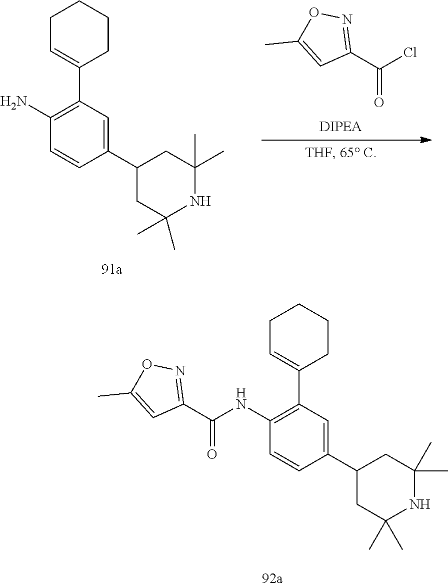

Compound 91a (30 mg, 0.096 mmol) was dissolved in Tetrahydrofuran (0.48 mL), DIPEA (33.4 μL, 0.192 mmol) was added thereto, and 5-methylisoxazole-3-carbonyl chloride (30 mg, 0.144 mmol) was added dropwise thereto, and the result was stirred for 2 hours. After confirming the completion of the reaction, the reaction mixture was extracted with MC:MeOH=10:1 and washed with water and brine. After drying with Anhydrous Na2SO4, the mixture was concentrated. Separation and purification were performed by column chromatography to obtain compound 92a (2 mg, 0.0047 mmol, 5%).

1H NMR (400 MHz, DMSO-d6) δ 9.59 (s, 1H), 7.75-7.68 (m, 1H), 7.18 (d, J=8.3 Hz, 1H), 7.13 (s, 1H), 6.63 (s, 1H), 5.70 (s, 1H), 3.22 (s, 1H), 2.49-2.49 (m, 3H), 2.21 (s, 2H), 2.11 (s, 2H), 1.95 (d, J=13.0 Hz, 1H), 1.79 (d, J=12.1 Hz, 2H), 1.65 (dd, J=26.8, 4.7 Hz, 6H), 1.42 (d, J=28.0 Hz, 12H).

HRMS(ESI+) calculated for C26H36N3O2 [M+H]+: 422.2802, found 422.9147.

### 5-methyl-N-(4′-methyl-5-(2,2,6,6-tetramethylpiperidin-4-yl)-2′,3′,4,5′-tetrahydro-[1,1′-biphenyl]-2-yl)isoxazole-3-carboxamide. (92b)

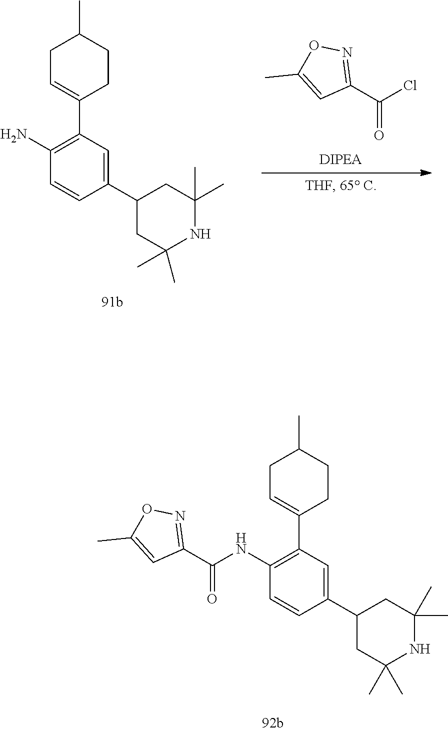

The reaction mixture was synthesized in the same manner as above, and separated and purified through column chromatography to obtain 15 mg (0.0344 mmol) of Compound 92b (Yield: 40%).

1H NMR (400 MHz, DMSO-d6) δ 9.56 (s, 1H), 7.69 (dd, J=11.4, 8.3 Hz, 1H), 7.16 (dd, J=8.3, 1.9 Hz, 1H), 7.09 (d, J=1.9 Hz, 1H), 6.64 (d, J=0.8 Hz, 1H), 5.67 (s, 1H), 3.03 (t, J=12.7 Hz, 1H), 2.51 (s, 3H), 2.33 (dd, J=10.8, 2.8 Hz, 1H), 2.22 (d, J=13.9 Hz, 2H), 1.98 (d, J=14.1 Hz, 1H), 1.81-1.70 (m, 3H), 1.61 (dd, J=12.6, 2.8 Hz, 2H), 1.29 (d, J=12.6 Hz, 2H), 1.24 (d, J=4.9 Hz, 7H), 1.08 (s, 6H), 1.00 (d, J=6.0 Hz, 3H).

HRMS(ESI+) calculated for C27H38N3O2 [M+H]+: 436.2959, found 436.5636.

### N-(4′,4′-dimethyl-5-(2,2,6,6-tetramethylpiperidin-4-yl)-2,3′,4′,5′-tetrahydro-[1,1′-biphenyl]-2-yl)-5-methylisoxazole-3-carboxamide. (92c)

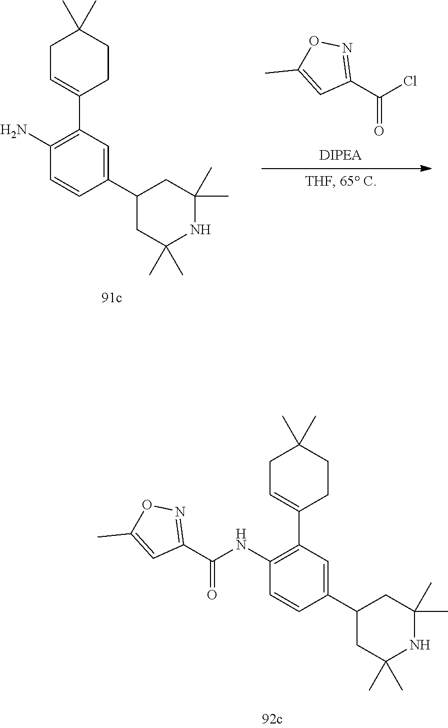

The reaction mixture was synthesized in the same manner as above, and separated and purified through column chromatography to obtain 20 mg (0.0445 mmol) of Compound 92c (Yield: 52%).

1H NMR (400 MHz, MeOD) δ 8.01 (d, J=8.4 Hz, 1H), 7.24 (dd, J=8.4, 2.1 Hz, 1H), 7.15 (d, J=2.1 Hz, 1H), 6.53 (d, J=0.8 Hz, 1H), 5.70 (d, J=3.7 Hz, 1H), 3.26 (dd, J=12.8, 3.1 Hz, 1H), 2.51 (d, J=0.6 Hz, 3H), 2.31 (d, J=2.0 Hz, 2H), 2.02 (d, J=3.3 Hz, 2H), 1.97 (dd, J=14.3, 3.1 Hz, 2H), 1.74 (t, J=13.5 Hz, 2H), 1.58 (s, 6H), 1.55 (d, J=6.4 Hz, 2H), 1.48 (s, 6H), 1.04 (d, J=8.5 Hz, 6H).

HRMS(ESI+) calculated for C28H40N3O2 [M+H]+: 450.3115, found 450.1043.

### N-(2-(cyclohept-1-en-1-yl)-4-(2,2,6,6-tetramethylpiperidin-4-yl)phenyl)-5-methylisoxazole-3-carboxamide. (92d)

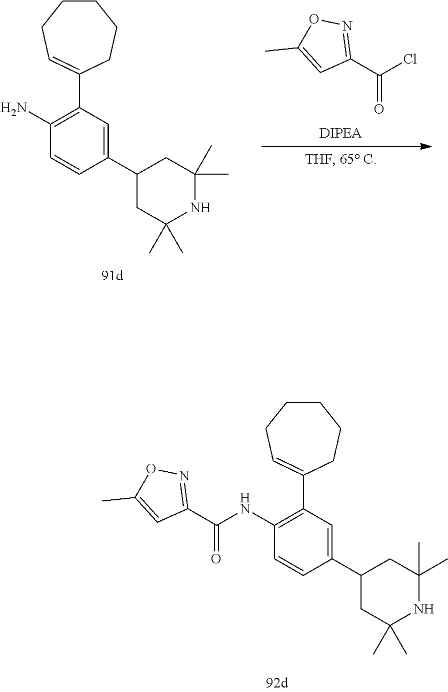

The reaction mixture was synthesized in the same manner as above, and separated and purified through column chromatography to obtain 5 mg (0.0115 mmol) of Compound 92d (Yield: 10%).

1H NMR (400 MHz, DMSO-d6) δ 9.02 (s, 1H), 7.98 (d, J=8.3 Hz, 1H), 7.13 (dd, J=8.5, 1.7 Hz, 1H), 6.97 (d, J=1.6 Hz, 1H), 6.67 (d, J=4.5 Hz, 1H), 5.38 (t, J=6.4 Hz, 1H), 3.19 (s, 1H), 2.50 (s, 3H), 2.36 (s, 2H), 1.95 (d, J=13.9 Hz, 2H), 1.74 (s, 6H), 1.65 (s, 2H), 1.55 (s, 2H), 1.47 (s, 7H), 1.41 (s, 6H).

HRMS(ESI+) calculated for C27H38N3O2 [M+H]+: 436.2959, found 436.8014.

### Experiment

In this Experimental Example, in order to confirm the activity and kinase selectivity of CSF-1R with respect to the synthesized compound, the Kinase HotSpotSM service (www.reactionbiology.com) of Reaction Biology Corp. was used for the IC50 identification of all compounds and kinase profiles. The assay protocol is polu [glu:Tyr]5 μM, ATP 10 μM and FMS (h) (5-10 mU) at a final reaction volume of 25 μL, 25 mM Tris (pH 7.5), 0.02mMEGTA, 0.66 mg/mL myelin basic protein, 10 mMMg acetate and [−33P-ATP] (inactive drug). Incubate with 500 cpm/pmol, concentration required). The reaction was initiated by the addition of a Mg-ATP mixture. After incubation at room temperature for 40 minutes, the reaction was stopped by adding 5 L of 3% phosphoric acid solution. Then, 10 L of the reaction was spotted on a P30 filter mat and washed 3 times in 75 mM phosphoric acid for 5 minutes and 1 time with methanol before drying and scintillation counting.

First, the results of confirming the activity of CSF-1R of the synthesized representative compounds are shown in Table 3 below (IC50<100 nM: ++, 1 μM>IC50>100 nM: ++, IC50>1 μM: +).

TABLE 3

| No | Compound No. | 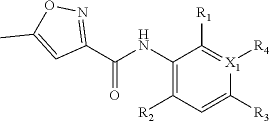 | IC₅₀ (M) on FMS |
|---|---|---|---|
| 1 | 5a | 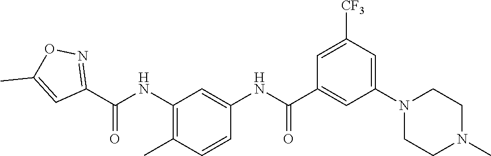 | +++ |
| 2 | 5b | 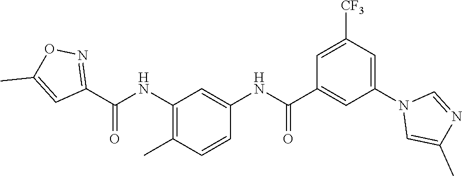 [SMILES: Cc1cn(-c2cc(C(=O)Nc3ccc(C)c(NC(=O)c4cc(C)on4)c3)cc(C(F)(F)F)c2)cn1] | +++ |
| 3 | 5c | 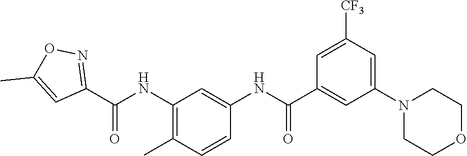 [SMILES: Cc1cc(C(=O)Nc2cc(NC(=O)c3cc(N4CCOCC4)cc(C(F)(F)F)c3)ccc2C)no1] | +++ |
| 4 | 5d | 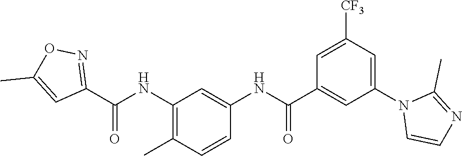 [SMILES: Cc1cc(C(=O)Nc2cc(NC(=O)c3cc(-n4ccnc4C)cc(C(F)(F)F)c3)ccc2C)no1] | +++ |
| 5 | 5e | 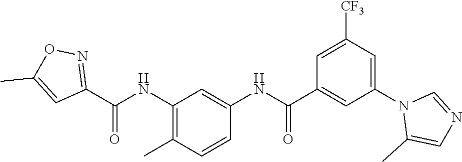 [SMILES: Cc1cc(C(=O)Nc2cc(NC(=O)c3cc(-n4cncc4C)cc(C(F)(F)F)c3)ccc2C)no1] | +++ |
| 6 | 5f | 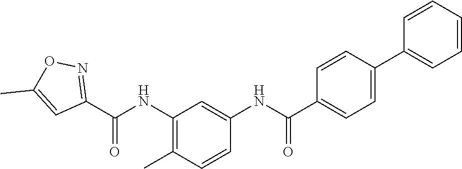 [SMILES: Cc1cc(C(=O)Nc2cc(NC(=O)c3ccc(-c4ccccc4)cc3)ccc2C)no1] | + |
| 7 | 5g | 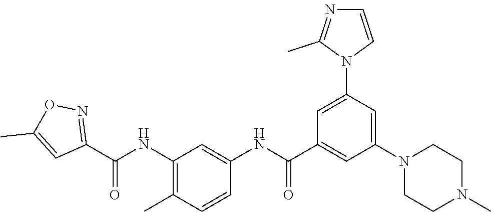 | + |
| 8 | 5h | 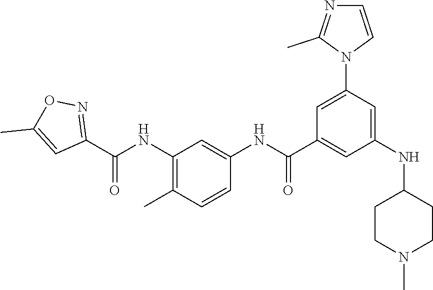 [SMILES: Cc1cc(C(=O)Nc2cc(NC(=O)c3cc(NC4CCN(C)CC4)cc(-n4ccnc4C)c3)ccc2C)no1] | + |
| 9 | 5i | 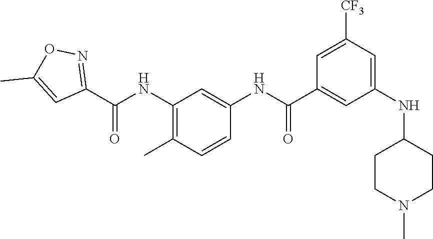 [SMILES: Cc1cc(C(=O)Nc2cc(NC(=O)c3cc(NC4CCN(C)CC4)cc(C(F)(F)F)c3)ccc2C)no1] | +++ |
| 10 | 5j | 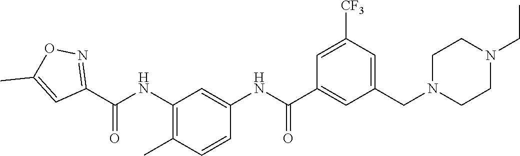 [SMILES: CCN1CCN(Cc2cc(C(=O)Nc3ccc(C)c(NC(=O)c4cc(C)on4)c3)cc(C(F)(F)F)c2)CC1] | ++ |
| 11 | 5k | 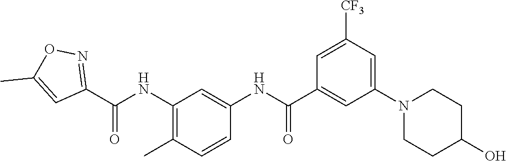 [SMILES: Cc1cc(C(=O)Nc2cc(NC(=O)c3cc(N4CCC(O)CC4)cc(C(F)(F)F)c3)ccc2C)no1] | ++ |
| 12 | 5l | 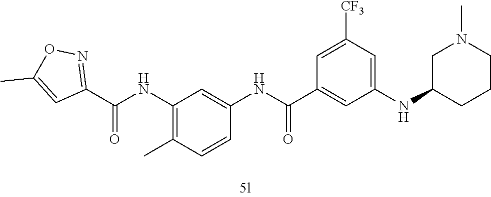 [SMILES: Cc1cc(C(=O)Nc2cc(NC(=O)c3cc(N[C@@H]4CCCN(C)C4)cc(C(F)(F)F)c3)ccc2C)no1] | + |
| 13 | 5m | 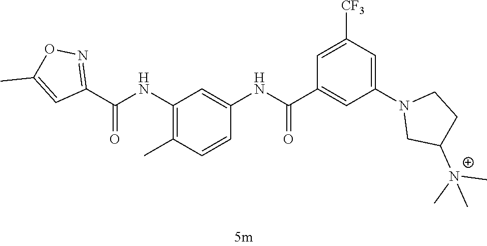 | + |
| 14 | 5n | 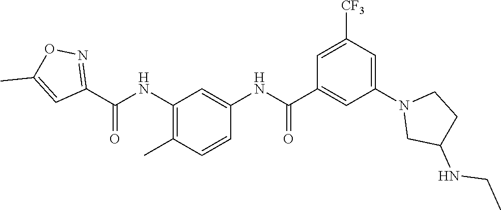 [SMILES: CCNC1CCN(c2cc(C(=O)Nc3ccc(C)c(NC(=O)c4cc(C)on4)c3)cc(C(F)(F)F)c2)C1] | ++ |
| 15 | 5o | 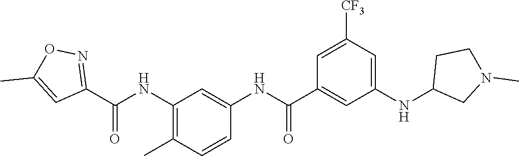 | ++ |
| 16 | 5p | 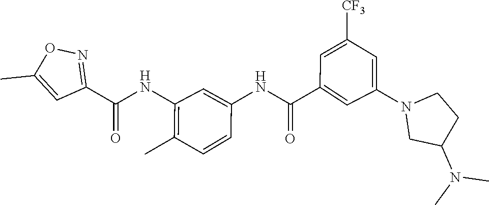 | ++ |
| 17 | 5q | 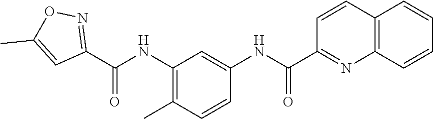 [SMILES: Cc1cc(C(=O)Nc2cc(NC(=O)c3ccc4ccccc4n3)ccc2C)no1] | + |
| 18 | 5r | 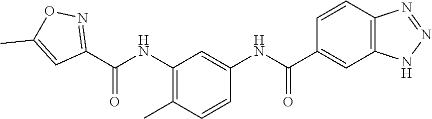 [SMILES: Cc1cc(C(=O)Nc2cc(NC(=O)c3ccc4nn[nH]c4c3)ccc2C)no1] | + |
| 19 | 5s | 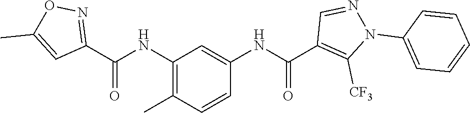 [SMILES: Cc1cc(C(=O)Nc2cc(NC(=O)c3cnn(-c4ccccc4)c3C(F)(F)F)ccc2C)no1] | + |
| 20 | 5t | 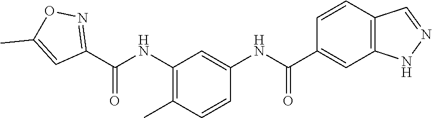 [SMILES: Cc1cc(C(=O)Nc2cc(NC(=O)c3ccc4cn[nH]c4c3)ccc2C)no1] | + |
| 21 | 5u | 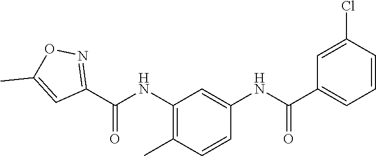 [SMILES: Cc1cc(C(=O)Nc2cc(NC(=O)c3cccc(Cl)c3)ccc2C)no1] | + |
| 22 | 5v | 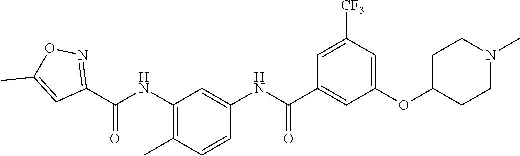 | +++ |
| 23 | 17b | 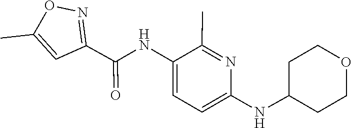 [SMILES: Cc1cc(C(=O)Nc2ccc(NC3CCOCC3)nc2C)no1] | +++ |
| 24 | 60a | 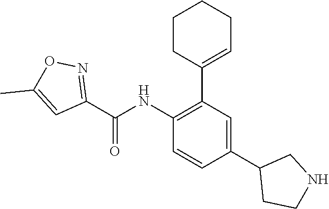 [SMILES: Cc1cc(C(=O)Nc2ccc(C3CCNC3)cc2C2=CCCCC2)no1] | ++ |
| 25 | 60b | 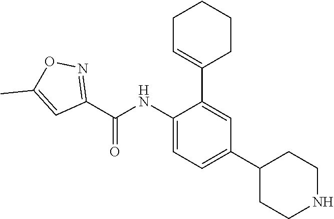 [SMILES: Cc1cc(C(=O)Nc2ccc(C3CCNCC3)cc2C2=CCCCC2)no1] | +++ |
| 26 | 60c | 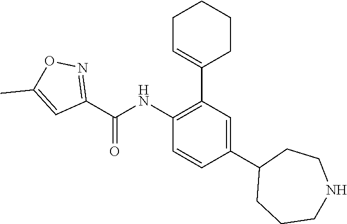 [SMILES: Cc1cc(C(=O)Nc2ccc(C3CCCNCC3)cc2C2=CCCCC2)no1] | +++ |
| 27 | 61b | 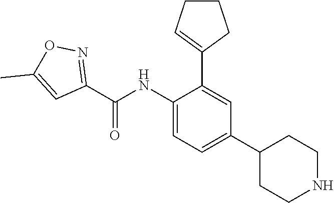 [SMILES: Cc1cc(C(=O)Nc2ccc(C3CCNCC3)cc2C2=CCCC2)no1] | + |
| 28 | 62b | 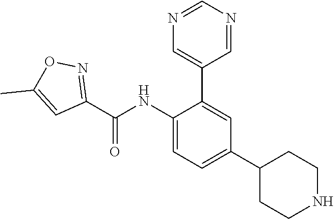 [SMILES: Cc1cc(C(=O)Nc2ccc(C3CCNCC3)cc2-c2cncnc2)no1] | + |
| 29 | 63b | 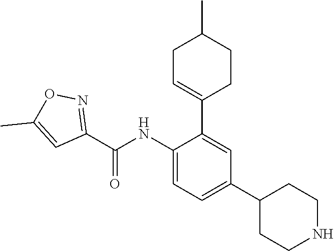 [SMILES: Cc1cc(C(=O)Nc2ccc(C3CCNCC3)cc2C2=CCC(C)CC2)no1] | +++ |
| 30 | 64b | 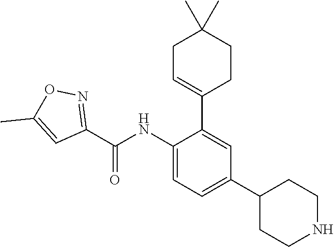 [SMILES: Cc1cc(C(=O)Nc2ccc(C3CCNCC3)cc2C2=CCC(C)(C)CC2)no1] | +++ |
| 31 | 64c | 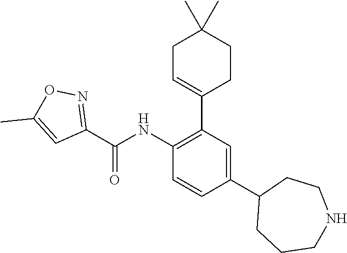 [SMILES: Cc1cc(C(=O)Nc2ccc(C3CCCNCC3)cc2C2=CCC(C)(C)CC2)no1] | +++ |
| 32 | 65b | 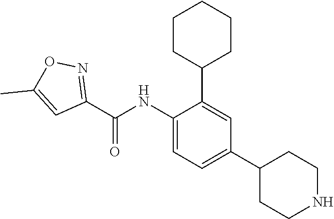 [SMILES: Cc1cc(C(=O)Nc2ccc(C3CCNCC3)cc2C2CCCCC2)no1] | + |
| 33 | 66b | 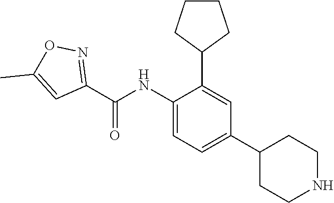 [SMILES: Cc1cc(C(=O)Nc2ccc(C3CCNCC3)cc2C2CCCC2)no1] | + |
| 34 | 67b | 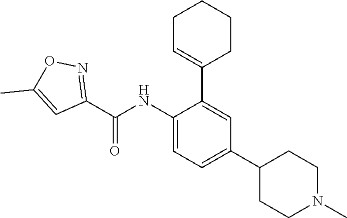 | +++ |
| 35 | 67c | 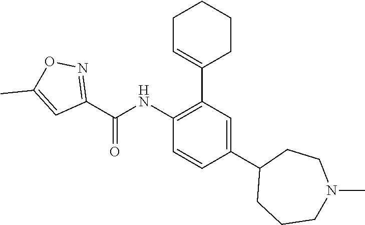 | ++ |
| 36 | 68c | 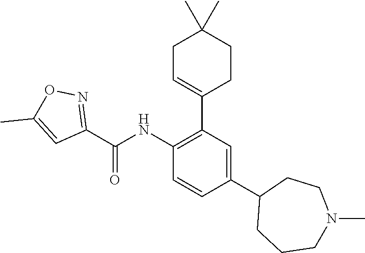 | +++ |
| 37 | 69b | 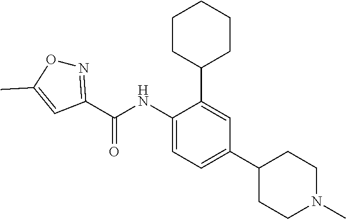 | + |
| 38 | 70b | 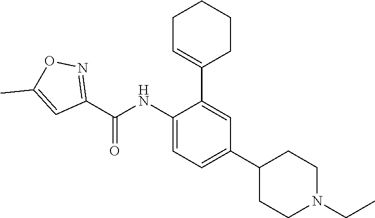 [SMILES: CCN1CCC(c2ccc(NC(=O)c3cc(C)on3)c(C3=CCCCC3)c2)CC1] | +++ |
| 39 | 71b | 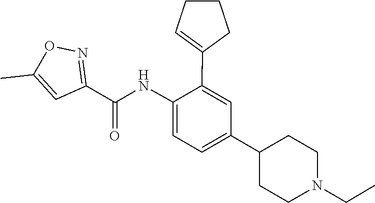 [SMILES: CCN1CCC(c2ccc(NC(=O)c3cc(C)on3)c(C3=CCCC3)c2)CC1] | + |
| 40 | 72b | 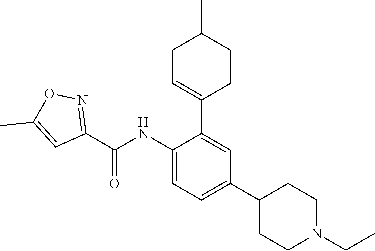 [SMILES: CCN1CCC(c2ccc(NC(=O)c3cc(C)on3)c(C3=CCC(C)CC3)c2)CC1] | +++ |
| 41 | 73b | 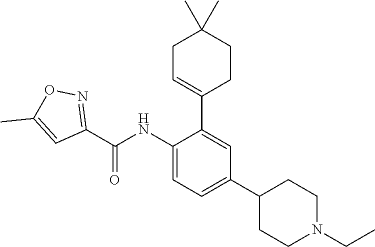 [SMILES: CCN1CCC(c2ccc(NC(=O)c3cc(C)on3)c(C3=CCC(C)(C)CC3)c2)CC1] | +++ |
| 42 | 74b | 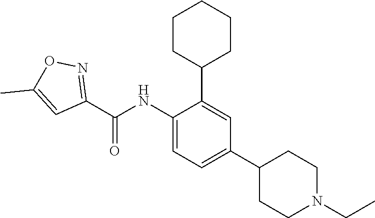 [SMILES: CCN1CCC(c2ccc(NC(=O)c3cc(C)on3)c(C3CCCCC3)c2)CC1] | + |
| 43 | 75b | 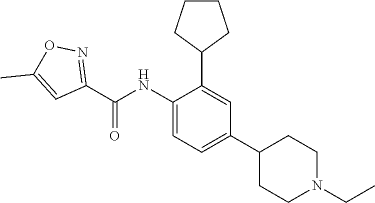 [SMILES: CCN1CCC(c2ccc(NC(=O)c3cc(C)on3)c(C3CCCC3)c2)CC1] | + |
| 44 | 76b | 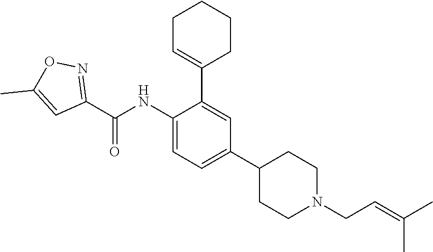 [SMILES: CC(C)=CCN1CCC(c2ccc(NC(=O)c3cc(C)on3)c(C3=CCCCC3)c2)CC1] | +++ |
| 45 | 77b | 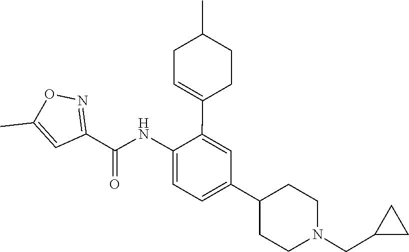 [SMILES: Cc1cc(C(=O)Nc2ccc(C3CCN(CC4CC4)CC3)cc2C2=CCC(C)CC2)no1] | +++ |
| 46 | 78b | 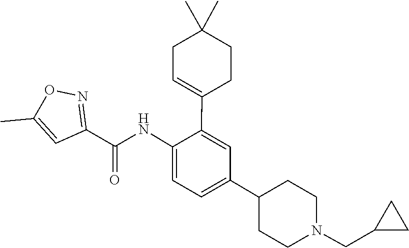 [SMILES: Cc1cc(C(=O)Nc2ccc(C3CCN(CC4CC4)CC3)cc2C2=CCC(C)(C)CC2)no1] | +++ |
| 47 | 79b | 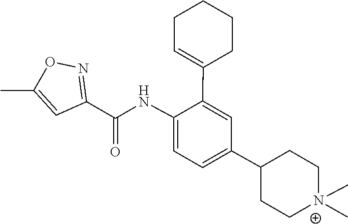 | +++ |
| 48 | 79c | 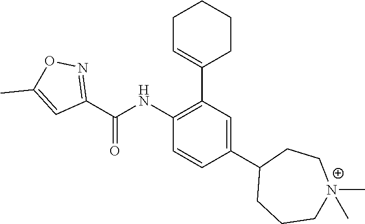 | +++ |
| 49 | 80c | 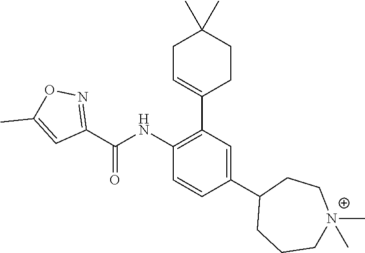 | +++ |
| 50 | 81b | 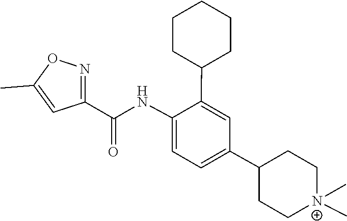 | ++ |
| 51 | 82b | 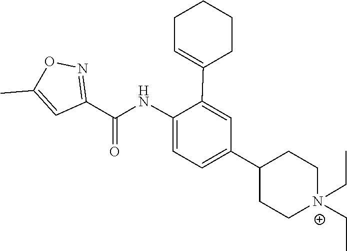 [SMILES: CC[N+]1(CC)CCC(c2ccc(NC(=O)c3cc(C)on3)c(C3=CCCCC3)c2)CC1] | +++ |
| 52 | 83b | 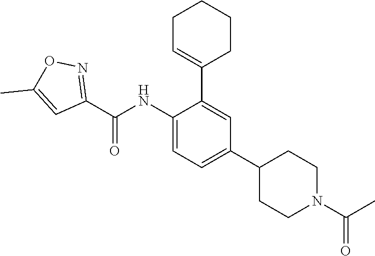 [SMILES: CC(=O)N1CCC(c2ccc(NC(=O)c3cc(C)on3)c(C3=CCCCC3)c2)CC1] | ++ |
| 53 | 84b | 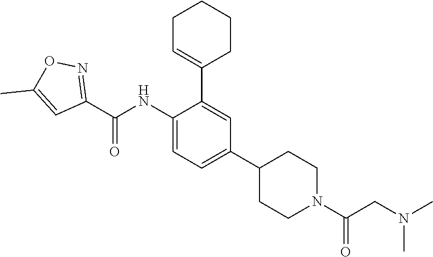 | ++ |
| 54 | 92a | 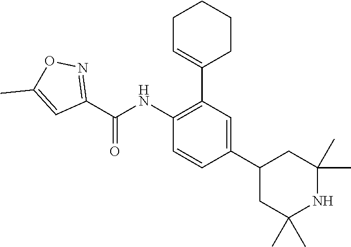 | +++ |
| 55 | 92b | 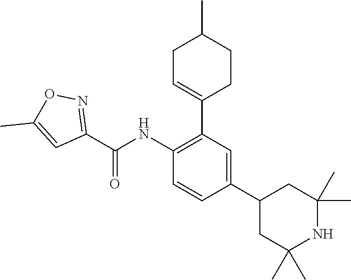 | +++ |
| 56 | 92c | 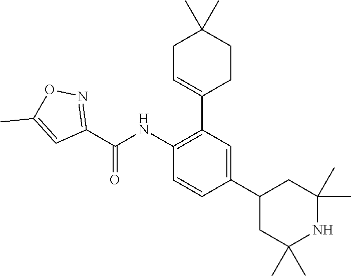 | +++ |
| 57 | 92d | 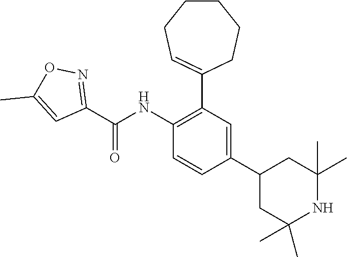 | ++ |

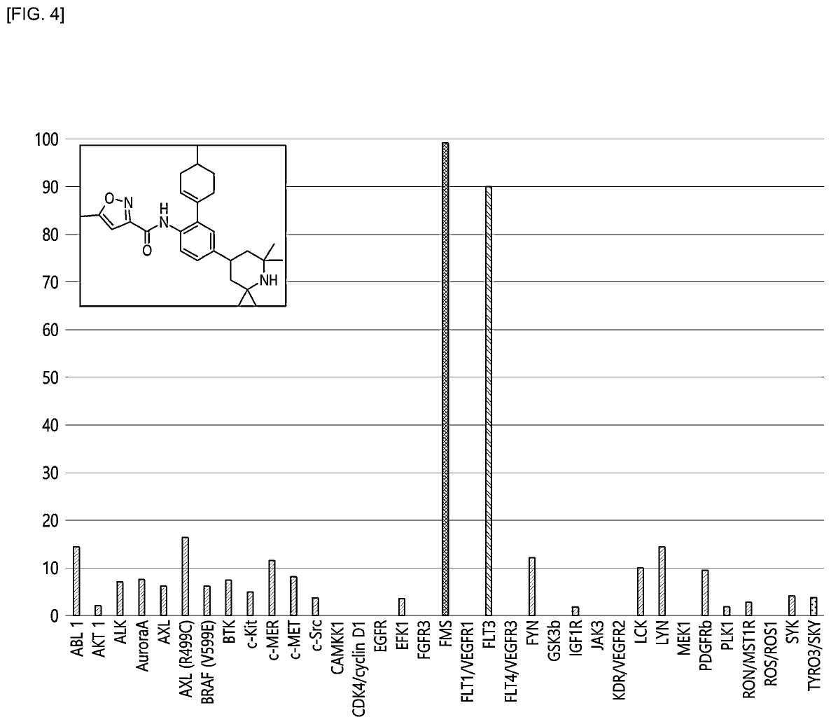

In addition, the results of confirming the kinase selectivity of EMS and FLT3 with respect to Compound 92b are shown in FIG. 4. The results confirmed high selectivity for EMS and FLT3 as shown in FIG. 4.

The present invention also confirmed that the position where the isoxazole moiety and the amide group are connected affects the stability of the compound and selectivity.

As a comparative example, 1, compound disclosed in “Journal of medical chemistry, Just Accepted Manual, 2018, Volume 61, pp. 5450-5466, Internal pp. 1-62 (2018.01.02)” was used and compared with compound 5a of the present invention. Table 4 below is the structural formula of the two compounds.

TABLE 4

| | |
|---|---|
| Example (compound 5a) | 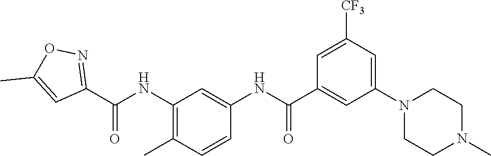 |
| Comparative example 1 | 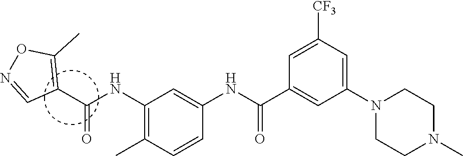 [SMILES: Cc1ccc(NC(=O)c2cc(N3CCN(C)CC3)cc(C(F)(F)F)c2)cc1NC(=O)c1cnoc1C] |

Referring to the structures of two compound, the isoxazole moiety and the amide group are connected at different positions.

Table 5 below is the analysis result of stabilities of the two compounds.

TABLE 5

| | | PAMPA assay | | MS | PS (human) | |
|---|---|---|---|---|---|---|
| | IC50 on | Pe (1*10−6 cm/sec) | | (human) | @30 | [symbol] @60 |
| Compound | CSF-1R | Avg | SD | %@60 min | min | min |
| Example | 4.67E−07 | 55.50 | 6.59 | 56.6 | >100 | >100 |
| (compound 5a) | | | | | | |
| Comparative | 1.03E−08 | ND | ND | <10 | 93.9 | 73.3 |
| example 1 | | | | | | |

Referring to the above result, vulnerabilities in metabolic stability (MS) and plasma stability (PS) of the 5-methylisooxazole moiety in Comparative example 1 are found in the above result. In particular, the acidic properties of 3-H of 5-methylisooxazole easily opened the ring in the presence of a base or enzyme to form 2-cyano-3-hydroxybut-2-enamide. Therefore, the present invention found the problem of such instability caused by the binding position of isoxazole moiety and the amide group and resolve the problem by changing the position of the isoxazole moiety. In other words, the fact that pharmaceutical stability depends upon the position of the isoxazole moiety is a new fact that has not been disclosed in the prior art.

Furthermore, to confirm the protein Kinase profile according to the direction (i.e., binding position) of isoxazole, the tendency was confirmed by comparing Compound 5d of the present invention with Comparative example 2.

TABLE 6
is the structural formula of the two compounds.

| | |
|---|---|
| Example (compound 5d) | 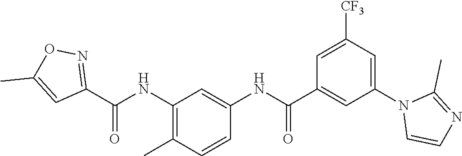 [SMILES: Cc1cc(C(=O)Nc2cc(NC(=O)c3cc(-n4ccnc4C)cc(C(F)(F)F)c3)ccc2C)no1] |
| Comparative example 2 | 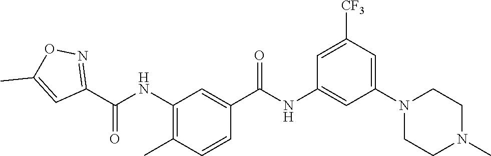 |

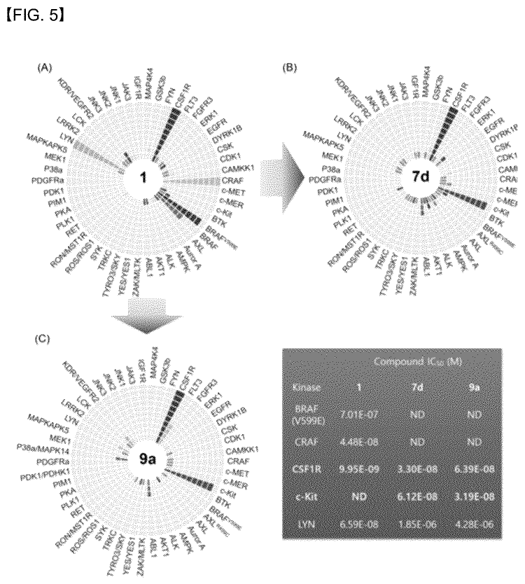

FIG. 5 is a graph showing the change of the protein kinase profile for 50 selected protein Kinase in Comparative example 1 (“1” in FIG. 5), Example 5d of the present invention (“7d” in FIG. 5), and Comparative example 2 (“9a” in FIG. 5), and the interpolated table is the quantitative data.

Referring to the above results, when treated at single-dose concentration 1.0 μM, the compound of Comparative Example 1 also demonstrated inhibitory activity against CRaf at 1 μM concentration, but also showed inhibitory activity against BRafV600E and Lyn in addition to CSF-1R.

However, 7d (Compound 5a of the present invention) and 9a (Comparative Example 2) in which the binding position of isoxazole is the same as that of the present invention showed excellent selectivity in inhibiting CSF-1R. Therefore, the significant change in selectivity due to the change of the hinge binder proves the remarkable effect according to the isoxazole position of the pharmaceutical compound according to the present invention.

As described above, specific parts of the present invention have been described in detail, and it will be apparent to those of ordinary skill in the art that such specific techniques are merely preferred embodiments, and the scope of the present invention is not limited thereby. Therefore, it will be said that the substantial scope of the present invention is defined by the accompanying claims and equivalents thereof.

### INDUSTRIAL APPLICABILITY

The present invention relates to an N-Aryl-5-methylisoxazole-3-carboxamide derivative or a pharmaceutically acceptable salt thereof, and a composition for preventing or treating CSF-1R-related diseases containing the derivative as an active ingredient. The N-Aryl-5-methylisoxazole-3-carboxamide of the present invention inhibits CSF-1R which regulates macrophages that drive neurodegenerative diseases, and thus can be useful in the prevention, alleviation, or treatment of neurodegenerative diseases such as Alzheimer's dementia, Parkinson's disease, Huntington's disease, and the like. brain diseases.

1. An isoxazole derivative or a pharmaceutically acceptable salt thereof represented by Chemical Formula 1: 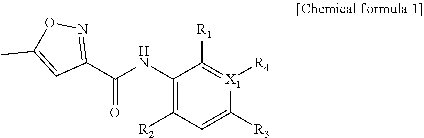
In Chemical Formula 1, X₁ is carbon or nitrogen, R₁ is a C₁ to C₆ linear or cyclic alkyl group, a C₆ to C₁₂ heteroaryl group, a substituted or unsubstituted C₄ to C₈ cycloalkene group, or hydrogen, R₂ is a C₁ to C₆ alkyl group or hydrogen, R₃ is a C₄ to C₈ heterocycloamine, a substituted or unsubstituted C₄ to C₈ heterocyclic ring, or hydrogen, R₄ is 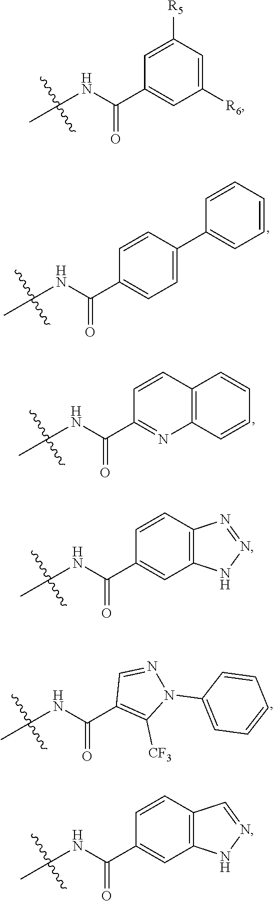
or hydrogen, R₅ is —CF₃, a substituted or unsubstituted C₃ to C₁₂ heteroaryl group, or halogen, and R₆ is a substituted or unsubstituted C₃ to C₁₂ aryl group, a substituted or unsubstituted C₃ to C₁₂ heteroaryl group, a substituted or unsubstituted C₃ to C₈ heterocyclic ring, a substituted C₄ to C₆ heterocyclooxy group, a substituted C₁ to C₆ alkyl group, or a substituted heterocycloamine group.

2. The isoxazole derivative or the pharmaceutically acceptable salt according to claim 1, wherein, in the Chemical Formula 1, R₁ is —CH₃, 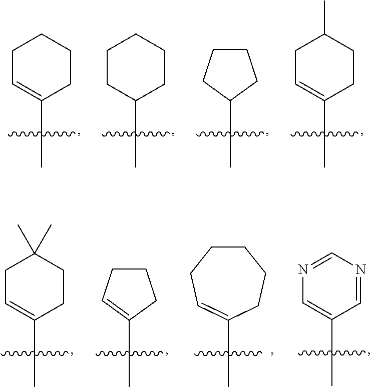
or hydrogen.

3. The isoxazole derivative or the pharmaceutically acceptable salt according to claim 1, wherein, the heterocyclic ring in the Chemical Formula 1 is one or more selected from the group consisting of piperazine, piperidine, piperidinium, pyrrolidine, azepan and morpholine, and the heteroaryl is imidazole or pyrimidine.

4. The isoxazole derivative or the pharmaceutically acceptable salt thereof according to claim 1, wherein a compound represented by the Chemical Formula 1 is at least one selected from groups consisting of 5-methyl-N-(2-methyl-5-(3-(4-methylpiperazin-1-yl)-5-(trifluoromethyl)benzamido)phenyl)isoxazole-3-carboxamide(5a);
5-methyl-N-(2-methyl-5-(3-(4-methyl-1H-imidazol-1-yl)-5-(trifluoromethyl)benzamido) phenyl)isoxazole-3-carboxamide(5b);
5-methyl-N-(2-methyl-5-(3-morpholino-5-(trifluoromethyl)benzamido)phenyl)isoxazole-3-carboxamide(5c);
5-methyl-N-(2-methyl-5-(3-(2-methyl-1H-imidazol-1-yl)-5-(trifluoromethyl)benzamido) phenyl)isoxazole-3-carboxamide(5d);
5-methyl-N-(2-methyl-5-(3-(5-methyl-1H-imidazol-1-yl)-5-(trifluoromethyl)benzamido) phenyl)isoxazole-3-carboxamide(5e);
N-(5-([1,1′-biphenyl]-4-carboxamido)-2-methylphenyl)-5-methylisoxazole-3-carboxamide(5f);
5-methyl-N-(2-methyl-5-(3-(2-methyl-1H-imidazol-1-yl)-5-(4-methylpiperazin-1-yl)benzamido) phenyl)isoxazole-3-carboxamide(5g);
5-methyl-N-(2-methyl-5-(3-(2-methyl-1H-imidazol-1-yl)-5-((1-methylpiperidin-4-yl)amino) benzamido)phenyl)isoxazole-3-carboxamide(5h);
5-methyl-N-(2-methyl-5-(3-((1-methylpiperidin-4-yl)amino)-5-(trifluoromethyl)benzamido) phenyl)isoxazole-3-carboxamide, 5i;
N-(5-(3-((4-Ethylpiperazin-1-yl)methyl)-5-(trifluoromethyl)benzamido)-2-methylphenyl)-5-methylisoxazole-3-carboxamide(5j);
N-(5-(3-(4-Hydroxypiperidin-1-yl)-5-(trifluoromethyl)benzamido)-2-methylphenyl)-5-methylisoxazole-3-carboxamide(5k);
(R)-5-Methyl-N-(2-methyl-5-(3-((1-methylpiperidin-3-yl)amino)-5-(trifluoromethyl)benzamido) phenyl)isoxazole-3-carboxamide(5l);
N,N,N-trimethyl-1-(3-((4-methyl-3-(5-methylisoxazole-3-carboxamido)phenyl)carbamoyl)-5-(trifluoromethyl)phenyl)pyrrolidin-3-aminium(5m);
N-(5-(3-(3-(ethylamino)pyrrolidin-1-yl)-5-(trifluoromethyl)benzamido)-2-methylphenyl)-5-methylisoxazole-3-carboxamide(5n);
5-methyl-N-(2-methyl-5-(3-((1-methylpyrrolidin-3-yl)amino)-5-(trifluoromethyl)benzamido)phenyl)isoxazole-3-carboxamide(5o);
N-(5-(3-(3-(dimethylamino)pyrrolidin-1-yl)-5-(trifluoromethyl)benzamido)-2-methylphenyl)-5-methylisoxazole-3-carboxamide(5p);
5-methyl-N-(2-methyl-5-(quinoline-2-carboxamido)phenyl)isoxazole-3-carboxamide(5q);
N-(5-(1H-benzo[d][1,2,3]triazole-6-carboxamido)-2-methylphenyl)-5-methylisoxazole-3-carboxamide(5r);
5-methyl-N-(2-methyl-5-(1-phenyl-5-(trifluoromethyl)-1H-pyrazole-4-carboxamido)phenyl)isoxazole-3-carboxamide(5s);
(N-(5-(1H-indazole-6-carboxamido)-2-methylphenyl)-5-methylisoxazole-3-carboxamide(5t);
N-(5-(3-Chlorobenzamido)-2-methylphenyl)-5-methylisoxazole-3-carboxamide(5u);
5-Methyl-N-(2-methyl-5-(3-((1-methylpiperidin-4-yl)oxy)-5-(trifluoromethyl)benzamido) phenyl)isoxazole-3-carboxamide(5v);
5-methyl-N-(2-methyl-6-((tetrahydro-2H-pyran-4-yl)amino)pyridin-3-yl)isoxazole-3-carboxamide(17b);
5-methyl-N-(5-(pyrrolidin-3-yl)-2′,3′,4′,5′-tetrahydro-[1,1′-biphenyl]-2-yl)isoxazole-3-carboxamide(60a);
5-methyl-N-(5-(piperidin-4-yl)-2′,3′,4′,5′-tetrahydro-[1,1′-biphenyl]-2-yl)isoxazole-3-carboxamide(60b);
N-(5-(azepan-4-yl)-2′,3′,4′,5′-tetrahydro-[1,1′-biphenyl]-2-yl)-5-methylisoxazole-3-carboxamide(60c);
N-(2-(Cyclopent-1-en-1-yl)-4-(piperidin-4-yl)phenyl)-5-methylisoxazole-3-carboxamide(61 b);
5-methyl-N-(4-(piperidin-4-yl)-2-(pyrimidin-5-yl)phenyl)isoxazole-3-carboxamide(62b);
5-Methyl-N-(4′-methyl-5-(piperidin-4-yl)-2′,3′,4′,5′-tetrahydro-[1,1′-biphenyl]-2-yl)isoxazole-3-carboxamide(63b);
N-(4′,4′-dimethyl-5-(piperidin-4-yl)-2′,3′,4′,5′-tetrahydro-[1,1′-biphenyl]-2-yl)-5-methylisoxazole-3-carboxamide(64b);
N-(5-(azepan-4-yl)-4′,4′-dimethyl-2′,3′,4′,5′-tetrahydro-[1,1′-biphenyl]-2-yl)-5-methylisoxazole-3-carboxamide(64c);
N-(2-cyclohexyl-4-(piperidin-4-yl)phenyl)-5-methylisoxazole-3-carboxamide(65b);
N-(2-Cyclopentyl-4-(piperidin-4-yl)phenyl)-5-methylisoxazole-3-carboxamide(66b);
5-methyl-N-(5-(1-methylpiperidin-4-yl)-2′,3′,4′,5′-tetrahydro-[1,1′-biphenyl]-2-yl)isoxazole-3-carboxamide(67b);
5-methyl-N-(5-(1-methylazepan-4-yl)-2′,3′,4′,5′-tetrahydro-[1,1′-biphenyl]-2-yl)isoxazole-3-carboxamide(67c);
N-(4′,4′-dimethyl-5-(1-methylazepan-4-yl)-2′,3′,4′,5′-tetrahydro-[1,1′-biphenyl]-2-yl)-5-methylisoxazole-3-carboxamide(68c);
N-(2-cyclohexyl-4-(1-methylpiperidin-4-yl)phenyl)-5-methylisoxazole-3-carboxamide(69b);
N-(5-(1-ethylpiperidin-4-yl)-2′,3′,4′,5′-tetrahydro-[1,1′-biphenyl]-2-yl)-5-methylisoxazole-3-carboxamide(70b);
N-(2-(Cyclopent-1-en-1-yl)-4-(1-ethylpiperidin-4-yl)phenyl)-5-methylisoxazole-3-carboxamide(71 b);
N-(5-(1-Ethylpiperidin-4-yl)-4′-methyl-2′,3′,4′,5′-tetrahydro-[1,1′-biphenyl]-2-yl)-5-methylisoxazole-3-carboxamide(72b);
N-(5-(1-ethylpiperidin-4-yl)-4′,4′-dimethyl-2′,3′,4′,5′-tetrahydro-[1,1′-biphenyl]-2-yl)-5-methylisoxazole-3-carboxamide(73b);
N-(2-cyclohexyl-4-(1-ethylpiperidin-4-yl)phenyl)-5-methylisoxazole-3-carboxamide(74b);
N-(2-Cyclopentyl-4-(1-ethylpiperidin-4-yl)phenyl)-5-methylisoxazole-3-carboxamide(75b);
5-methyl-N-(5-(1-(3-methylbut-2-en-1-yl)piperidin-4-yl)-2′,3′,4′,5′-tetrahydro-[1,1′-biphenyl]-2-yl)isoxazole-3-carboxamide(76b);
N-(5-(1-(cyclopropylmethyl)piperidin-4-yl)-4′-methyl-2′,3′,4′,5′-tetrahydro-[1,1′-biphenyl]-2-yl)-5-methylisoxazole-3-carboxamide(77b);
N-(5-(1-(cyclopropylmethyl)piperidin-4-yl)-4′,4′-dimethyl-2′,3′,4′,5′-tetrahydro-[1,1′-biphenyl]-2-yl)-5-methylisoxazole-3-carboxamide(78b);
1,1-dimethyl-4-(6-(5-methylisoxazole-3-carboxamido)-2′,3′,4′,5′-tetrahydro-[1,1′-biphenyl]-3-yl)piperidin-1-ium(79b);
1,1-dimethyl-4-(6-(5-methylisoxazole-3-carboxamido)-2′,3′,4′,5′-tetrahydro-[1,1′-biphenyl]-3-yl)azepan-1-ium(79c);
4-(4′,4′-dimethyl-6-(5-methylisoxazole-3-carboxamido)-2′,3′,4′,5′-tetrahydro-[1,1′-biphenyl]-3-yl)-1,1-dimethylazepan-1-ium(80c);
4-(3-cyclohexyl-4-(5-methylisoxazole-3-carboxamido)phenyl)-1,1-dimethylpiperidin-1-ium(81b);
1,1-diethyl-4-(6-(5-methylisoxazole-3-carboxamido)-2′,3′,4′,5′-tetrahydro-[1,1′-biphenyl]-3-yl)piperidin-1-ium(82b);
N-(5-(1-acetylpiperidin-4-yl)-2′,3′,4′,5′-tetrahydro-[1,1′-biphenyl]-2-yl)-5-methylisoxazole-3-carboxamide(83b);
N-(5-(1-(dimethylglycyl)piperidin-4-yl)-2′,3′,4′,5′-tetrahydro-[1,1′-biphenyl]-2-yl)-5-methylisoxazole-3-carboxamide(84b);
5-methyl-N-(5-(2,2,6,6-tetramethylpiperidin-4-yl)-2′,3′,4′,5′-tetrahydro-[1,1′-biphenyl]-2-yl)isoxazole-3-carboxamide(92a);
5-methyl-N-(4′-methyl-5-(2,2,6,6-tetramethylpiperidin-4-yl)-2′,3′,4′,5′-tetrahydro-[1,1′-biphenyl]-2-yl)isoxazole-3-carboxamide(92b);
N-(4′,4′-dimethyl-5-(2,2,6,6-tetramethylpiperidin-4-yl)-2′,3′,4′,5′-tetrahydro-[1,1′-biphenyl]-2-yl)-5-methylisoxazole-3-carboxamide, 92c; and N-(2-(cyclohept-1-en-1-yl)-4-(2,2,6,6-tetramethylpiperidin-4-yl)phenyl)-5-methylisoxazole-3-carboxamide(92d).

5. A pharmaceutical composition for preventing or treating a disease related to the colony stimulating factor 1 receptor comprising the compound of claim 1 or a pharmaceutically acceptable salt thereof as an active ingredient.

6. A pharmaceutical composition for preventing or treating a disease related to the colony stimulating factor 1 receptor comprising the compound of claim 2 or a pharmaceutically acceptable salt thereof as an active ingredient.

7. A pharmaceutical composition for preventing or treating a disease related to the colony stimulating factor 1 receptor comprising the compound of claim 3 or a pharmaceutically acceptable salt thereof as an active ingredient.

8. A pharmaceutical composition for preventing or treating a disease related to the colony stimulating factor 1 receptor comprising the compound of claim 4 or a pharmaceutically acceptable salt thereof as an active ingredient.

9. The pharmaceutical composition for preventing or treating a disease related to the colony stimulating factor 1 receptor according to claim 5, wherein the composition inhibits the colony stimulating factor 1 receptor.

10. The pharmaceutical composition for preventing or treating a disease related to the colony stimulating factor 1 receptor according to claim 9, wherein the disease is a degenerative brain disease.

11. The pharmaceutical composition for preventing or treating a disease related to the colony stimulating factor 1 receptor according to claim 10, wherein the degenerative brain disease is at least one selected from the group consisting of Alzheimer's disease, Parkinson's disease, Huntington's disease, prosencephaly, microcephaly, cerebral palsy, congenital hydrocephalus, Wilson disease, dementias, multi infarct dementia, Frontotemporal dementia, pseudo-dementia, Motor neuron diseases, spinocerebellar ataxia, spinal muscular atrophy.

12. The pharmaceutical composition for preventing or treating a disease related to the colony stimulating factor 1 receptor according to claim 6, wherein said composition inhibits the colony stimulating factor 1 receptor.

13. The pharmaceutical composition for preventing or treating a disease related to the colony stimulating factor 1 receptor according to claim 6, wherein the disease is a degenerative brain disease.

14. The pharmaceutical composition for preventing or treating a disease related to the colony stimulating factor 1 receptor according to claim 7, wherein said composition inhibits the colony stimulating factor 1 receptor.

15. The pharmaceutical composition for preventing or treating a disease related to colony stimulating factor 1 receptor according to claim 7, wherein the disease is a degenerative brain disease.

16. A method for preventing or treating a disease related to colony stimulating factor 1 receptor comprising administering the compound of claim 1 or a pharmaceutically acceptable salt thereof to an individual.

17. A method for preventing or treating a disease related to colony stimulating factor 1 receptor comprising administering the compound of claim 2 or a pharmaceutically acceptable salt thereof to an individual.

18. A method for preventing or treating a disease related to colony stimulating factor 1 receptor comprising administering the compound of claim 3 or a pharmaceutically acceptable salt thereof to an individual.

19. A method for preventing or treating a disease related to colony stimulating factor 1 receptor comprising administering the compound of claim 4 or a pharmaceutically acceptable salt thereof to an individual.

20. The method for preventing or treating a disease related to colony stimulating factor 1 receptor according to claim 16, wherein the disease is a degenerative brain disease.
